(19) United States
(12) Patent Application Publication
(10) Pub. No.: US 2004/0038307 A1
(43) Pub. Date: Feb. 26, 2004

(54) Unique recognition sequences and methods of use thereof in protein analysis

(75) Inventors: Frank D. Lee, Chestnut Hill, MA (US); Xun Meng, Newton, MA (US); John W. Chan, Acton, MA (US); Shengsheng Zhang, Quincy, MA (US); Stephen J. Benkovic, State College, PA (US)

Correspondence Address: ROPES & GRAY LLP, ONE INTERNATIONAL PLACE, BOSTON, MA, 02110-2624, US

(73) Assignee: engeneOS, Inc., Waltham, MA

(21) Appl. No.: 10/436,549

(22) Filed: May 12, 2003

Related U.S. Application Data
(60) Provisional application No. 60/379,626, filed on May 10, 2002.
(60) Provisional application No. 60/393,137, filed on Jul. 1, 2002.
(60) Provisional application No. 60/393,233, filed on Jul. 1, 2002.
(60) Provisional application No. 60/393,235, filed on Jul. 1, 2002.
(60) Provisional application No. 60/393,211, filed on Jul. 1, 2002.
(60) Provisional application No. 60/393,223, filed on Jul. 1, 2002.
(60) Provisional application No. 60/393,280, filed on Jul. 1, 2002.
(60) Provisional application No. 60/393,197, filed on Jul. 1, 2002.
(60) Provisional application No. 60/430,948, filed on Dec. 4, 2002.
(60) Provisional application No. 60/433,319, filed on Dec. 13, 2002.

(51) Int. Cl. C12Q 1/68; G01N 33/53; G06F 19/00; G01N 33/48; G01N 33/50

(52) U.S. Cl. 435/7.1; 435/6; 702/19

(57) ABSTRACT

Disclosed are methods for reliably detecting the presence of proteins in a sample by the use of capture agents that recognize and interact with recognition sequences uniquely characteristic of a set of proteins in the sample. Arrays comprising these capture agents are also provided.

### RELATED APPLICATIONS

This application claims priority to U.S. Provisional Application No. 60/379,626, filed on May 10, 2002; U.S. Provisional Application Nos. 60/393,137, 60/393,233, 60/393,235, 60/393,211, 60/393,223, 60/393,280, and 60/393,197, all filed on Jul. 1, 2002; U.S. Provisional Application No. 60/430,948, filed on Dec. 4, 2002; and U.S. Provisional Application No. 60/433319 filed on Dec. 13, 2002, the entire contents of each of which are incorporated herein by reference.

### BACKGROUND OF THE INVENTION

Genomic studies are now approaching “industrial” speed and scale, thanks to advances in gene sequencing and the increasing availability of high-throughput methods for studying genes, the proteins they encode, and the pathways in which they are involved. The development of DNA microarrays has enabled massively parallel studies of gene expression as well as genomic DNA variations.

DNA microarrays have shown promise in advanced medical diagnostics. More specifically, several groups have shown that when the gene expression patterns of normal and diseased tissues are compared at the whole genome level, patterns of expression characteristic of the particular disease state can be observed. Bittner et al., (2000) Nature 406:536-540; Clark et al., (2000) Nature 406:532-535; Huang et al., (2001) Science 294:870-875; and Hughes et al., (2000) Cell 102:109-126. For example, tissue samples from patients with malignant forms of prostate cancer display a recognizably different pattern of mRNA expression to tissue samples from patients with a milder form of the disease. C. f., Dhanasekaran et al., (2001) Nature 412 (2001), pp. 822-826.

However, as James Watson pointed out recently proteins are really the “actors in biology” (“A Cast of Thousands” Nature Biotechnology March 2003). A more attractive approach would be to monitor key proteins directly. These might be biomarkers identified by DNA microarray analysis. In this case, the assay required might be relatively simple, examining only 5-10 proteins. Another approach would be to use an assay that detects hundreds or thousands of protein features, such as for the direct analysis of blood, sputum or urine samples, etc. It is reasonable to believe that the body would react in a specific way to a particular disease state and produce a distinct “biosignature” in a complex data set, such as the levels of 500 proteins in the blood. One could imagine that in the future a single blood test could be used to diagnose most conditions.

The motivation for the development of large-scale protein detection assays as basic research tools is different to that for their development for medical diagnostics. The utility of biosignatures is one aspect researchers desire in order to understand the molecular basis of cellular response to a particular genetic, physiological or environmental stimulus. DNA microarrays do a good job in this role, but detection of proteins would allow for more accurate determination of protein levels and, more importantly, could be designed to quantitate the presence of different splice variants or isoforms. These events, to which DNA microarrays are largely or completely blind, often have pronounced effects on protein activities.

This has sparked great interest in the development of devices such as protein-detecting microarrays (PDMs) to allow similar experiments to be done at the protein level, particularly in the development of devices capable of monitoring the levels of hundreds or thousands of proteins simultaneously.

Prior to the present invention, PDMs that even approach the complexity of DNA microarrays do not exist. There are several problems with the current approaches to massively parallel, e.g., cell-wide or proteome wide, protein detection. First, reagent generation is difficult: One needs to first isolate every individual target protein in order to isolate a detection agent against every protein in an organism and then develop detection agents against the purified protein. Since the number of proteins in the human organism is currently estimated to be about 30,000 this requires a lot of time (years) and resources. Furthermore, detection agents against native proteins have less defined specificity since it is a difficult task to know which part of the proteins the detection agents recognize. This problem causes considerable cross-reactivity of when multiple detection agents are arrayed together, making large-scale protein detection array difficult to construct. Second, current methods achieve poor coverage of all possible proteins in an organism. These methods typically include only the soluble proteins in biological samples. They often fail to distinguish splice variants, which are now appreciated as being ubiquitous. They exclude a large number of proteins that are bound in organellar and cellular membranes or are insoluble when the sample is processed for detection. Third, current methods are not general to all proteins or to all types of biological samples. Proteins vary quite widely in their chemical character. Groups of proteins require different processing conditions in order to keep them stably solubilized for detection. Any one condition may not suit all the proteins. Further, biological samples vary in their chemical character. Individual cells considered identical express different proteins over the course of their generation and ultimate death. Physiological fluids like urine and blood serum are relatively simple, but biopsy tissue samples are very complex. Different protocols need to be used to process each type of sample and achieve maximal solubilization and stabilization of proteins.

Current detection methods are either not effective over all proteins uniformly or cannot be highly multiplexed to enable simultaneous detection of a large number of proteins (e.g., >5,000). Optical detection methods would be most cost effective but suffer from lack of uniformity over different proteins. Proteins in a sample have to be labeled with dye molecules and the different chemical character of proteins leads to inconsistency in efficiency of labeling. Labels may also interfere with the interactions between the detection agents and the analyte protein leading to further errors in quantitation. Non-optical detection methods have been developed but are quite expensive in instrumentation and are very difficult to multiplex for parallel detection of even moderately large samples (e.g., >100 samples).

Another problem with current technologies is that they are burdened by intracellular life processes involving a complex web of protein complex formation, multiple enzymatic reactions altering protein structure, and protein conformational changes. These processes can mask or expose binding sites known to be present in a sample. For example, prostate specific antigen (PSA) is known to exist in serum in multiple forms including free (unbound) forms, e.g., pro-PSA, BPSA (BPH-associated free PSA), and complexed forms, e.g., PSA-ACT, PSA-A2M (PSA-alpha₂-macroglobulin), and PSA-API (PSA-alpha₁-protease inhibitor) (see Stephan C. et al. (2002) Urology 59:2-8). Similarly, Cyclin E is known to exist not only as a full length 50 kD protein, but also in five other low molecular weight forms ranging in size from 34 to 49 kD. In fact, the low molecular weight forms of cyclin E are believed to be more sensitive markers for breast cancer than the full length protein (see Keyomarsi K. et al. (2002) N. Eng. J. Med. 347(20):1566-1575).

Sample collection and handling prior to a detection assay may also affect the nature of proteins that are present in a sample and, thus, the ability to detect these proteins. As indicated by Evans M. J. et al. (2001) Clinical Biochemistry 34:107-112 and Zhang D. J. et al. (1998) Clinical Chemistry 44(6):1325-1333, standarizing immunoassays is difficult due to the variability in sample handling and protein stability in plasma or serum. For example, PSA sample handling, such as sample freezing, affects the stability and the relative levels of the different forms of PSA in the sample (Leinonen J, Stenman U H (2000) Tumour Biol. 21(1):46-53).

Finally, current technologies are burdened by the presence of autoantibodies which affect the outcome of immunoassays in unpredictable ways, e.g., by leading to analytical errors (Fitzmaurice T. F. et al. (1998) Clinical Chemistry 44(10):2212-2214).

These problems prompted the question whether it is even possible to standardize immunoassays for hetergenous protein antigens. (Stenman U -H. (2001) Immunoassay Standardization: Is it possible? Who is responsible? Who is capable? Clinical Chemistry 47 (5) 815-820). Thus, a great need exists in the art for efficient and simple methods of parallel detection of proteins that are expressed in a biological sample and, particularly, for methods that can overcome the imprecisions caused by the complexity of protein chemistry and for methods which can detect all or a majority of the proteins expressed in a given cell type at a given time, or for proteome-wide detection and quantitation of proteins expressed in biological samples.

### SUMMARY OF THE INVENTION

The present invention is directed to methods and reagents for reproducible protein detection and quantitation, e.g., parallel detection and quantitation, in complex biological samples. Salient features to certain embodiments of the present invention reduce the complexity of reagent generation, achieve greater coverage of all protein classes in an organism, greatly simplify the sample processing and analyte stabilization process, and enable effective and reliable parallel detection, e.g., by optical or other automated detection methods, and quantitation of proteins and/or post-translationally modified forms, and, enable multiplexing of standardized capture agents for proteins with minimal cross-reactivity and well-defined specificity for large-scale, proteome-wide protein detection.

Embodiments of the present invention also overcome the imprecisions in detection methods caused by: the existence of proteins in multiple forms in a sample (e.g., various post-translationally modified forms or various complexed or aggregated forms); the variability in sample handling and protein stability in a sample, such as plasma or serum; and the presence of autoantibodies in samples. In certain embodiments, using a targeted fragmentation protocol, the methods of the present invention assure that a binding site on a protein of interest, which may have been masked due to one of the foregoing reasons, is made available to interact with a capture agent. In other embodiments, the sample proteins are subjected to conditions in which they are denatured, and optionally are alkylated, so as to render buried (or otherwise cryptic) URS moieties accessible to solvent and interaction with capture agents. As a result, the present invention allows for detection methods having increased sensitivity and more accurate protein quantitation capabilities. This advantage of the present invention will be particularly useful in, for example, protein marker-type disease detection assays (e.g., PSA or Cyclin E based assays) as it will allow for an improvement in the predictive value, sensitivity, and reproducibility of these assays. The present invention can standardize detection and measurement assays for all proteins from all samples.

The present invention is based, at least in part, on the realization that exploitation of unique recognition sequences (URSs) present within individual proteins can enable reproducible detection and quantitation of individual proteins in parallel in a milieu of proteins in a biological sample. As a result of this unique recognition sequence-based approach, the methods of the invention detect specific proteins in a manner that does not require preservation of the whole protein, nor even its native tertiary structure, for analysis. Moreover, the methods of the invention are suitable for the detection of most or all proteins in a sample, including insoluble proteins such as cell membrane bound and organelle membrane bound proteins.

The present invention is also based, at least in part, on the realization that unique recognition sequences can serve as Proteome Epitope Tags characteristic of a specific organism's proteome and can enable the recognition and detection of a specific organism.

The present invention is also based, at least in part, on the realization that high-affinity agents (such as antibodies) with predefined specificity can be generated for defined, short length peptides and when antibodies recognize protein or peptide epitopes, only 4-6 (on average) amino acids are critical. See, for example, Lerner R A (1984) Advances In Immunology. 36:1-45.

The present invention is also based, at least in part, on the realization that by denaturing and/or fragmenting all proteins in a sample to produce a soluble set of protein analytes, e.g., in which even otherwise buried URS's are solvent accessible, the subject method provides a reproducible and accurate (intra-assay and inter-assay) measurement of proteins.

Accordingly, in one aspect, the present invention provides a method for globally detecting the presence of a protein(s) (e.g., membrane bound protein(s)) in an organism's proteome. The method includes providing a sample which has been denatured and/or fragmented to generate a collection of soluble polypeptide analytes; contacting the polypeptide analytes with a plurality of capture agents (e.g., capture agents immobilized on a solid support such as an array) under conditions such that interaction of the capture agents with corresponding unique recognition sequences occurs, thereby globally detecting the presence of protein(s) in an organism's proteome.

The method is suitable for use in, for example, diagnosis (e.g., clinical diagnosis or environmental diagnosis), drug discovery, protein sequencing or protein profiling. In one embodiment, at least 50%, 55%, 60%, 65%, 70%, 75%, 80%, 85%, 90%, 95% or 100% of an organism's proteome is detectable from arrayed capture agents.

The capture agent may be a protein, a peptide, an antibody, e.g., a single chain antibody, an artificial protein, an RNA or DNA aptamer, an allosteric ribozyme, a small molecule or electronic means of capturing a URS.

The sample to be tested (e.g., a human, yeast, mouse, C. elegans, Drosophila melanogaster or Arabidopsis thaliana sample, such whole cell lysate) may be fragmented by the use of a proteolytic agent. The proteolytic agent can be any agent, which is capable of cleaving polypeptides between specific amino acid residues (i.e., the proteolytic cleavage pattern). According to one embodiment of this aspect of the present invention a proteolytic agent is a proteolytic enzyme. Examples of proteolytic enzymes, include but are not limited to trypsin, calpain, carboxypeptidase, chymotrypsin, V8 protease, pepsin, papain, subtilisin, thrombin, elastase, gluc-C, endo lys-C or proteinase K, caspase-1, caspase-2, caspase-3, caspase-4, caspase-5, caspase-6, caspase-7, caspase-8, MetAP-2, adenovirus protease, HIV protease and the like. According to another embodiment of this aspect of the present invention a proteolytic agent is a proteolytic chemical such as cyanogen bromide and 2-nitro-5-thiocyanobenzoate. In still other embodiments, the proteins of the test sample can be fragmented by physical shearing; by sonication, or some combination of these or other treatment steps.

An important feature for certain embodiments, particularly when analyzing complex samples, is to develop a fragmentation protocol that is known to reproducibly generate peptides, preferably soluble peptides, which serve as the unique recognition sequences.The collection of polypeptide analytes generated from the fragmentation may be 5-30, 5-20, 5-10, 10-20, 20-30, or 10-30 amino acids long, or longer. Ranges intermediate to the above recited values, e.g., 7-15 or 15-25 are also intended to be part of this invention. For example, ranges using a combination of any of the above recited values as upper and/or lower limits are intended to be included.

The unique recognition sequence may be a linear sequence or a non-contiguous sequence and may be 5, 6, 7, 8, 9, 10, 11, 12, 13, 14, 15, 16, 17, 18, 19, 20, 25, or 30 amino acids in length. In certain embodiments, the unique recognition sequence is selected from the group consisting of SEQ ID NOs:1-546 or a sub-collection thereof.

In one embodiment, the protein(s) being detected is characteristic of a pathogenic organism, e.g., anthrax, small pox, cholera toxin, Staphylococcus aureus α-toxin, Shiga toxin, cytotoxic necrotizing factor type 1, Escherichia coli heat-stable toxin, botulinum toxins, or tetanus neurotoxins.

In another aspect, the present invention provides a method for detecting the presence of a protein, preferably simultaneous or parallel detection of multiple proteins, in a sample. The method includes providing a sample which has been denatured and/or fragmented to generate a collection of soluble polypeptide analytes; providing an array comprising a support having a plurality of discrete regions to which are bound a plurality of capture agents, wherein each of the capture agents is bound to a different discrete region and wherein each of the capture agents is able to recognize and interact with a unique recognition sequence within a protein; contacting the array of capture agents with the polypeptide analytes; and determining which discrete regions show specific binding to the sample, thereby detecting the presence of a protein in a sample.

To further illustrate, the present invention provides a packaged protein detection array. Such arrays may include an addressable array having a plurality of features, each feature independently including a discrete type of capture agent that selectively interacts with a unique recognition sequence (URS) of an analyte protein, e.g., under conditions in which the analyte protein is a soluble protein produced by proteolysis and/or denaturation. The features of the array are disposed in a pattern or with a label to provide the identity of interactions between analytes and the capture agents, e.g., to ascertain the the identity and/or quantity of a protein occurring in the sample. The packated array may also include instructions for (i) contacting the addressable array with a sample containing polypeptide analytes produced by denaturation and/or cleavage of proteins at amide backbone positions; (ii) detecting interaction of said polypeptide analytes with said capture agent moieties; (iii) and determining the identity of polypeptide analytes, or native proteins from which they are derived, based on interaction with capture agent moieties.

In yet a further aspect, the present invention provides a method for detecting the presence of a protein in a sample by providing a sample which has been denatured and/or fragmented to generate a collection of soluble polypeptide analytes; contacting the sample with a plurality of capture agents, wherein each of the capture agents is able to recognize and interact with a unique recognition sequence within a protein, under conditions such that the presence of a protein in the sample is detected.

In another aspect, the present invention provides a method for detecting the presence of a protein in a sample by providing an array of capture agents comprising a support haying a plurality of discrete regions (features) to which are bound a plurality of capture agents, wherein each of the capture agents is bound to a different discrete region and wherein the plurality of capture agents are capable of interacting with at least 50% of an organism's proteome; contacting the array with the sample; and determining which discrete regions show specific binding to the sample, thereby detecting the presence of a protein in the sample.

In a further aspect, the present invention provides a method for globally detecting the presence of a protein(s) in an organism's proteome by providing a sample comprising the protein and contacting the sample with a plurality of capture agents under conditions such that interaction of the capture agents with corresponding unique recognition sequences occurs, thereby globally detecting the presence of protein(s) in an organism's proteome.

In another aspect, the present invention provides a plurality of capture agents, herein the plurality of capture agents are capable of interacting with at least 50%, 55%, 60%, 65%, 70%, 75%, 80%, 85%, 90%, 95% or 100% of an organism's proteome and wherein each of the capture agents is able to recognize and interact with a unique recognition sequence within a protein.

In yet another aspect, the present invention provides an array of capture agents, which includes a support having a plurality of discrete regions to which are bound a plurality of capture agents (, e.g., at least 10, 20, 30, 40, 50, 60, 70, 80, 90, 100, 200, 300, 400, 500, 1000, 2000, 3000, 4000, 5000, 6000, 7000, 8000, 9000, 10000, 11000, 12000 or 13000 different capture agents), wherein each of the capture agents is bound to a different discrete region and wherein each of the capture agents is able to recognize and interact with a unique recognition sequence within a protein. The capture agents may be attached to the support, e.g., via a linker, at a density of 50, 100, 150,,200, 250, 300, 350, 400, 450, 500 or 1000 capture agents/cm². In one embodiment, each of the discrete regions is physically separated from each of the other discrete regions.

The capture agent array can be produced on any suitable solid surface, including silicon, plastic, glass, polymer, such as cellulose, polyacrylamide, nylon, polystyrene, polyvinyl chloride or polypropylene, ceramic, photoresist or rubber surface. Preferably, the silicon surface is a silicon dioxide or a silicon nitride surface. Also preferably, the array is made in a chip format. The solid surfaces may be in the form of tubes, beads, discs. silicon chips, microplates, polyvinylidene difluoride (PVDF) membrane, nitrocellulose membrane, nylon membrane, other purous membrane, non-porous membrane, e.g., plastic, polymer, perspex, silicon, amongst others, a plurality of polymeric pins, or a plurality of microtitre wells, or any other surface suitable for immobilizing proteins and/or conducting an immunoassay or other binding assay.

The capture agent may be a protein, a peptide, an antibody, e.g., a single chain antibody, an artificial protein, an RNA or DNA aptamer, an allosteric ribozyme or a small molecule.

In a further aspect, the present invention provides a composition comprising a plurality of isolated unique recognition sequences, wherein the unique recognition sequences are derived from at least 50%, 55%, 60%, 65%, 70%, 75%, 80%, 85%, 90%, 95% or 100% of an organism's proteome. In one embodiment, each of the unique recognition sequences is derived from a different protein.

In another aspect, the present invention provides a method for preparing an array of capture agents. The method includes providing a plurality of isolated unique recognition sequences, the plurality of unique recognition sequences derived from at least 50%, 55%, 60%, 65%, 70%, 75%, 80%, 85%, 90%, 95% or 100% of an organism's proteome; generating a plurality of capture agents capable of binding the plurality of unique recognition sequences; and attaching the plurality of capture agents to a support having a plurality of discrete regions, wherein each of the capture agents is bound to a different discrete region, thereby preparing an array of capture agents.

In one fundamental aspect, the invention provides an apparatus for detecting simultaneously the presence of plural specific proteins in a multi-protein sample, e.g., a body fluid sample or a cell sample produced by lysing a natural tissue sample or miroroorganism sample. The apparatus comprises a plurality of immobilized capture agents for contact with the sample and which include at least a subset of agents which respectively bind specifically with individual unique recognition sequences, and means for detecting binding events between respective capture agents and the unique recognition sequences, e.g., probes for detecting the presence and/or concentration of unique recognition sequences bound to the capture agents. The unique recognition sequences are selected such that the presence of each sequence is unambiguously indicative of the presence in the sample (before it is fragmented) of a target protein from which it was derived. Each sample is treated with a set proteolytic protocol so that the unique recognition sequences are generated reproducibly. Optionally, the means for detecting binding events may include means for detecting data indicative of the amount of bound unique recognition sequence. This permits assessment of the relative quantity of at least two target proteins in said sample.

The invention also provides methods for simultaneously detecting the presence of plural specific proteins in a multi-protein sample. The method comprises denaturing and/or fragmenting proteins in a sample using a predetermined protocol to generate plural unique recognition sequences, the presence of which in the sample are indicative unambiguously of the presence of target proteins from which they were derived. At least a portion of the Recognition Sequences in the sample are contacted with plural capture agents which bind specifically to at least a portion of the unique recognition sequences. Detection of binding events to particular unique recognition sequences indicate the presence of target proteins corresponding to those sequences.

In another aspect, the present invention provides methods for improving the reproducibility of protein binding assays conducted on biological samples. The improvement enables detecting the presence of the target protein with greater effective sensitivity, or quantitating the protein more reliably (i.e., reducing standard deviation). The methods include: (1) treating the sample using a pre-determined protocol which A) inhibits masking of the target protein caused by target protein-protein non covalent or covalent complexation or aggregation, target protein degradation or denaturing, target protein post-translational modification, or environmentally induced alteration in target protein tertiary structure, and B) fragments the target protein to, thereby, produce at least one peptide epitope (i.e., a URS) whose concentration is directly proportional to the true concentration of the target protein in the sample; (2) contacting the so treated sample with a capture agent for the URS under suitable binding conditions, and (3) detecting binding events qualitatively or quantitatively.

For certain embodiments of the subject assay, the capture agents that are made available according to the teachings herein can be used to develop multiplex assays having increased sensitivity, dynamic range and/or recovery rates relative to, for example ELISA and other immunoassays. Such improved performance characteristics can include one or more of the following: a regression coefficient (R2) of 0.95 or greater for a reference standard, e.g., a comparable control sample, more preferably an R2 greater than 0.97, 0.99 or even 0.995; an average recovery rate of at least 50 percent, and more preferably at least 60, 75, 80 or even 90 percent; a average positive predictive value for the occurrence of proteins in a sample of at least 90 percent, more preferably at least 95, 98 or even 99 percent; an average diagnostic sensitivity (DSN) for the occurrence of proteins in a sample of 99 percent or higher, more preferably at least 99.5 or even 99.8 percent; an average diagnostic specificity (DSP) for the occurrence of proteins in a sample of 99 percent or higher, more preferably at least 99.5 or even 99.8 percent.

Other features and advantages of the invention will be apparent from the following detailed description and claims.

### BRIEF DESCRIPTION OF THE DRAWINGS

FIG. 1 depicts the sequence of the Interleukin-8 receptor A and the pentamer unique recognition sequences (URS) within this sequence.

FIG. 2 depicts the sequence of the Histamine H1 receptor and the pentamer unique recognition sequences (URS) within this sequence that are not destroyed by trypsin digestion.

FIG. 3 is an alternative format for the parallel detection of URS from a complex sample. In this type of “virtual array” each of many different beads displays a capture agent directed against a different URS. Each different bead is color-coded by covalent linkage of two dyes (dye1 and dye2) at a characteristic ratio. Only two different beads are shown for clarity. Upon application of the sample, the capture agent binds a cognate URS, if present in the sample. Then a mixture of secondary binding ligands (in this case labeled URS peptides) conjugated to a third fluorescent tag is applied to the mixture of beads. The beads can then be analyzed using flow cytometry other detection method that can resolve, on a bead-by-bead basis, the ratio of dye1 and dye2 and thus identify the URS captured on the bead, while the fluorescence intensity of dye3 is read to quantitate the amount of labeled URS on the bead (which will in inversely reflect the analyte URS level).

FIG. 4 illustrates: a) a schematic drawing of fluorescence sandwich immunoassay for specific capture and quantitation of a targeted peptide in a complex peptide mixture; b) results of readout fluorescent signal detected by the secondary antibody.

### DETAILED DESCRIPTION OF THE INVENTION

The present invention provides methods, reagents and systems for detecting, e.g., globally detecting, the presence of a protein or a panel of proteins in a sample. In certain embodiments, the method may be used to quantitate the level of expression or post-translational modification of one or more proteins in the sample. The method includes providing a sample which has, preferably, been fragmented and/or denatured to generate a collection of peptides, and contacting the sample with a plurality of capture agents, wherein each of the capture agents is able to recognize and interact with a unique recognition sequence (URS) characteristic of a specific protein or modified state. Through detection and deconvolution of binding data, the presence and/or amout of a protein in the sample is determined.

In the first step, a biological sample is obtained. The biological sample as used herein refers to any body sample such as blood (serum or plasma), sputum, ascites fluids, pleural effusions, urine, biopsy specimens, isolated cells and/or cell membrane preparation. Methods of obtaining tissue biopsies and body fluids from mammals are well known in the art.

Retrieved biological samples can be further solubilized using detergent-based or detergent free (i.e., sonication) methods, depending on the biological specimen and the nature of the examined polypeptide (i.e., secreted, membrane anchored or intracellular soluble polypeptide).

In certain embodiments, the solubilized biological sample is contacted with one or more proteolytic agents. Digestion is effected under effective conditions and for a period of time sufficient to ensure complete digestion of the diagnosed polypeptide(s). Agents that are capable of digesting a biological sample under moderate conditions in terms of temperature and buffer stringency are preferred. Measures are taken not to allow nonspecific sample digestion, thus the quantity of the digesting agent, reaction mixture conditions (i.e., salinity and acidity), digestion time and temperature are carefully selected. At the end of incubation time proteolytic activity is terminated to avoid non-specific proteolytic activity, which may evolve from elongated digestion period, and to avoid further proteolysis of other peptide-based molecules (i.e., protein-derived capture agents), which are added to the mixture in following steps.

In the next method step the rendered biological sample is contacted with one or more capture agents, which are capable of discriminately binding one or more protein analytes through interaction via URS binding, and the products of such binding interactions examined and, as necessary, deconvolved, in order to identify and/or quantitate proteins found in the sample.

The present invention is based, at least in part, on the realization that unique recognition sequences (URSs), which can be identified by computional analysis, can characterize individual proteins in a given sample, e.g., identify a particular protein from amongst others and/or identify a particular post-translationally modified form of a protein. The use of agents that bind URSs can be exploitated for the detection and quantitation of individual proteins from a milieu of several or many proteins in a biological sample. The subject method can be used to assess the status of proteins in, for example, bodily fluids, cell or tissue samples, cell lystates, cell membranes, etc. In certain embodiments, the method utilizes a set of capture agents which discriminate between splice variants, allelic variants and/or point mutations (e.g., altered amino acid sequences arising from single nucleotide polymorphisms).

As a result of the sample preparation, namely denaturation and/or proteolysis, the subject method can be used to detect specific proteins in a manner that does not require the homogeneity of the target protein for analysis and is relatively refractory to small but otherwise significant differences between samples. The methods of the invention are suitable for the detection of all or any selected subset of all proteins in a sample, including cell membrane bound and organelle membrane bound proteins.

In certain embodiments, the detection step(s) of the method are not sensitive to post-translational modifications of the native protein; while in other embodiments, the preparation steps are designed to preserve a post-translational modification of interest, and the detection step(s) use a set of capture agents able to discriminate between modified and unmodified forms of the protein. Exemplary post-translational modifications that the subject method can be used to detect and quantitate include acetylation, amidation, deamidation, prenylation (such as farnesylation or geranylation), formylation, glycosylation, hydroxylation, methylation, myristoylation, phosphorylation, ubiquitination, ribosylation and sulphation. In one specific embodiment, the phosphorylation to be assessed is phosphorylation on tyrosine, serine, threonine or histidine residue. In another specific embodiment, the addition of a hydrophobic group to be assessed is the addition of a fatty acid, e.g., myristate or palmitate, or addition of a glycosyl-phosphatidyl inositol anchor. In certain embodiment, the present method can be used to assess protein modification profile of a particular disease or disorder, such as infection, neoplasm (neoplasia), cancer, an immune system disease or disorder, a metabolism disease or disorder, a muscle and bone disease or disorder, a nervous system disease or disorder, a signal disease or disorder, or a transporter disease or disorder.

As used herein, the term “unique recognition sequence” or “URS” is intended to mean an amino acid sequence that, when detected in a particular sample, unambiguously indicates that the protein from which it was derived is present in the sample. For instance, a URS is selected such that its presence in a sample, as indicated by detection of an authentic binding event with a capture agent designed to selectively bind with the sequence, necessarily means that the protein which comprises the sequence is present in the sample. A useful URS must present a binding surface that is solvent accessible when a protein mixture is denatured and/or fragmented, and must bind with significant specificity to a selected capture agent with minimal cross reactivity. A unique recognition sequence is is present within the protein from which it is derived and in no other protein that may be present in the sample, cell type, or species under investigation. Moreover, a URS will preferably not have any closely related sequence, such as determined by a nearest neighbor analysis, among the other proteins that may be present in the sample. A URS can be derived from a surface region of a protein, buried regions, splice junctions, or post translationally modified regions.

Perhaps the ideal URS is a peptide sequence which is present in only one protein in the proteome of a species. But a peptide comprising a URS useful in a human sample may in fact be present within the structure of proteins of other organisms. A URS useful in an adult cell sample is “unique” to that sample even though it may be present in the structure of other different proteins of the same organism at other times in its life, such as during embryology, or is present in other tissues or cell types different from the sample under investigation. A URS may be unique even though the same amino acid sequence is present in the sample from a different protein provided one or more of its amino acids are derivatized, and a binder can be developed which resolves the peptides.

When referring herein to “uniqueness” with respect to a URS, the reference is always made in relation to the foregoing. Thus, within the human genome, a URS may be an amino acid sequence that is truly unique to the protein from which it is derived. Alternatively, it may be unique just to the sample from which it is derived, but the same amino acid sequence may be present in, for example, the murine genome. Likewise, when referring to a sample which may contain proteins from multiple different organism, uniqueness refers to the ability to unambiguosly identify and discriminate between proteins from the different organisms, such as being from a host or from a pathogen.

Thus, a unique recognition sequence may be present within more than one protein in the species, provided it is unique to the sample from which it is derived. For example, a URS may be an amino acid sequence that is unique to: a certain cell type, e.g., a liver, brain, heart, kidney or muscle cell; a certain biological sample, e.g., a plasma, urine, amniotic fluid, genital fluid, marrow, spinal fluid, or pericardial fluid sample; a certain biological pathway, e.g., a G-protein coupled receptor signaling pathway or a tumor necrosis factor (TNF) signaling pathway.

The unique recognition sequence may be found in the native protein from which it is derived as a contiguous or as a non-contiguous amino acid sequence. It typically will comprise a portion of the sequence of a larger peptide or protein, recognizable by a capture agent either on the surface of an intact or partially degraded or digested protein, or on a fragment of the protein produced by a predetermined fragmentation protocol. The unique recognition sequence may be 5, 6, 7, 8, 9, 10, 11, 12, 13,14, 15, 16, 17, 18, 19 or 20 amino acid residues in length. In a preferred embodiment, the URS is 6, 7, 8, 9 or 10 amino acid residues in length.

The term “discriminate”, as in “capture agents able to discriminate between”, refers to a relative difference in the binding of a capture agent to its intended protein analyte and background binding to other proteins (or compounds) present in the sample. In particular, a capture agent can discriminate between two different species of proteins (or species of modifications) if the difference in binding constants is such that a statistically significant difference in binding is produced under the assay protocols and detection sensitivities. In preferred embodiments, the capture agent will have a discriminating index (D.I.) of at least 0.5, and even more preferably at least 0.1, 0.001, or even 0.0001, wherein D.I. is defined as K_{d}(a)/K_{d}(b), K_{d}(a) being the dissociation constant for the intended analyte, K_{d}(b) is the dissociation constant for any other protein (or modified form as the case may be) present in sample.

As used herein, the term “Proteome Epitope Tag” is intended to include the special collection of unique recognition sequences that characterize, and that are unique to, the proteome of a specific organism.

As used herein, the term “capture agent” includes any agent which is capable of binding to a protein that includes a unique recognition sequence, e.g., with at least detectable selectivity. A capture agent is capable of specifically interacting with (directly or indirectly), or binding to (directly or indirectly) a unique recognition sequence. The capture agent is preferably able to produce a signal that may be detected. In a preferred embodiment, the capture agent is an antibody or a fragment thereof, such as a single chain antibody, or a peptide selected from a displayed library. In other embodiments, the capture agent may be an artificial protein, an RNA or DNA aptamer, an allosteric ribozyme or a small molecule. In other embodiments, the capture agent may allow for electronic (e.g., computer-based or information-based) recognition of a unique recognition sequence. In one embodiment, the capture agent is an agent that is not naturally found in a cell.

As used herein, the term “globally detecting” includes detecting at least 40% of the proteins in the sample. In a preferred embodiment, the term “globally detecting” includes detecting at least 50%, 60%, 65%, 70%, 75%, 80%, 85%, 90%, 95% or 100% of the proteins in the sample. Ranges intermediate to the above recited values, e.g., 50%-70% or 75%-95%, are also intended to be part of this invention. For example, ranges using a combination of any of the above recited values as upper and/or lower limits are intended to be included.

As used herein, the term “proteome” refers to the complete set of chemically distinct proteins found in an organism.

As used herein, the term “organism” includes any living organism including animals, e.g., avians, insects, mammals such as humans, mice, rats, monkeys, or rabbits; microorganisms such as bacteria, yeast, and fungi, e.g., Escherichia coli, Campylobacter, Listeria, Legionella, Staphylococcus, Streptococcus, Salmonella, Bordatella, Pneumococcus, Rhizobium, Chlamydia, Rickettsia, Streptomyces, Mycoplasma, Helicobacter pylori, Chlamydia pneumoniae, Coxiella burnetii, Bacillus Anthracis, and Neisseria; protozoa, e.g., Trypanosoma brucei; viruses, e.g., human immunodeficiency virus, rhinoviruses, rotavirus, influenza virus, Ebola virus, simian immunodeficiency virus, feline leukemia virus, respiratory syncytial virus, herpesvirus, pox virus, polio virus, parvoviruses, Kaposi's Sarcoma-Associated Herpesvirus (KSHV), adeno-associated virus (AAV), Sindbis virus, Lassa virus, West Nile virus, enteroviruses, such as 23 Coxsackie A viruses, 6 Coxsackie B viruses, and 28 echoviruses, Epstein-Barr virus, caliciviruses, astroviruses, and Norwalk virus; fungi, e.g., Rhizopus, neurospora, yeast, or puccinia; tapeworms, e.g., Echinococcus granulosus, E. multilocularis, E. vogeli and E. oligarthrus; and plants, e.g., Arabidopsis thaliana, rice, wheat, maize, tomato, alfalfa, oilseed rape, soybean, cotton, sunflower or canola.

As used herein, “sample” refers to anything which may contain a protein analyte. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). The sample may also be a mixture of target protein containing molecules prepared in vitro;

As used herein, “a comparable control sample” refers to a control sample that is only different in one or more defined aspects relative to a test sample, and the present methods, kits or arrays are used to identify the effects, if any, of these defined difference(s) between the test sample and the control sample, e.g., on the amounts and types of proteins expressed and/or on the protein modification profile. For example, the control biosample can be derived from physiological normal conditions and/or can be subjected to different physical, chemical, physiological or drug treatments, or can be derived from different biological stages, etc.

A report by MacBeath and Schreiber (Science 289 (2000), pp. 1760-1763) in 2000 established that proteins could be printed and assayed in a microarray format, and thereby had a large role in renewing the excitement for the prospect of a protein chip. Shortly after this, Snyder and co-workers reported the preparation of a protein chip comprising nearly 6000 yeast gene products and used this chip to identify new classes of calmodulin- and phospholipid-binding proteins (Zhu et al., Science 293 (2001), pp. 2101-2105). The proteins were generated by cloning the open reading frames and overproducing each of the proteins as glutathione-S-transferase-(GST) and His-tagged fusions. The fusions were used to facilitate the purification of each protein and the His-tagged family were also used in the immobilization of proteins. This and other references in the art established that microarrays containing thousands of proteins could be prepared and used to discover binding interactions. They also reported that proteins immobilized by way of the His tag—and therefore uniformly oriented at the surface—gave superior signals to proteins randomly attached to aldehyde surfaces.

Related work has addressed the construction of antibody arrays (de Wildt et al., Antibody arrays for high-throughput screening of antibody-antigen interactions. Nat. Biotechnol. 18 (2000), pp. 989-994; Haab, B. B. et al. (2001) Protein microarrays for highly parallel detection and quantitation of specific proteins and antibodies in complex solutions. Genome Biol. 2, RESEARCH0004.1-RESEARCH0004.13). Specifically, in an early landmark report, de Wildt and Tomlinson immobilized phage libraries presenting scFv antibody fragments on filter paper to select antibodies for specific antigens in complex mixtures (supra). The use of arrays for this purpose greatly increased the throughput when evaluating antibodies, allowing nearly 20,000 unique clones to be screened in one cycle. Brown and co-workers extended this concept to create molecularly defined arrays wherein antibodies were directly attached to aldehyde-modified glass. They printed 115 commercially available antibodies and analyzed their interactions with cognate antigens with semi-quantitative results (supra). Kingsmore and co-workers used an analogous approach to prepare arrays of antibodies recognizing 75 distinct cytokines and, using the rolling-circle amplification strategy (Lizardi et al., Mutation detection and single molecule counting using isothermal rolling circle amplification. Nat. Genet. 19 (1998), pp. 225-233), could measure cytokines at femtomolar concentrations (Schweitzer et al., Multiplexed protein profiling on microarrays by rolling-circle amplification. Nat. Biotechnol. 20 (2002), pp. 359-365).

These examples demonstrate the many important roles that protein chips can play, and give evidence for the widespread activity in fabrication of these tools. The following subsections describes in further detail about various aspects of the invention.

I. Type of Capture Agents

In certain preferred embodiments, the capture agents used should be capable of selective affinity reactions with URS moieties. Generally, such ineraction will be non-covalent in nature, though the present invention also contemplates the use of capture reagents that become covalently linked to the URS.

Examples of capture agents which can be used include, but are not limited to: nucleotides; nucleic acids including oligonucleotides, double stranded or single stranded nucleic acids (linear or circular), nucleic acid aptamers and ribozymes; PNA (peptide micleic acids); proteins, including antibodies (such as monoclonal or recombinantly engineered antibodies or antibody fragments), T cell receptor and MHC complexes, lectins and scaffolded peptides; peptides; other naturally occurring polymers such as carbohydrates; artificial polymers, including plastibodies; small organic molecules such as drugs, metabolites and natural products; and the like.

In certain embodiments, the capture agents are immobilized, permanently or reversibly, on a solid support such as a bead, chip, or slide. When employed to analyze a complex mixture of proteins, the immobilized capture agent are arrayed and/or otherwise labeled for deconvolution of the binding data to yield identity of the capture agent (and therefore of the protein to which it binds) and (optionally) to quantitate binding. Alternatively, the capture agents can be provided free in solution (soluble), and other methods can be used for deconvolving URS binding in parallel.

In one embodiment, the capture agents are conjugated with a reporter molecule such as a fluorescent molecule or an enzyme, and used to detect the presence of bound URS on a substrate (such as a chip or bead), in for example, a “sandwich” type assay in which one capture agent is immobilized on a support to capture a URS, while a second, labeled capture agent also specific for the captured URS may be added to detect/quantitate the captured URS. In other embodiments a labeled-URS peptide is used in a competitive binding assay to determine the amount of unlabeled URS (from the sample) binds to the capture agent.

An important advantage of the invention is that useful capture agents can be identified and/or synthesized even in the absence of a sample of the protein to be detected. With the completion of the whole genome in a number of organisms, such as human, fly (Drosophila melanogaster) and nematode (C. elegans), URS of a given length or combination thereof can be identified for any single given protein in a certain organism, and capture agents for any of these proteins of interest can then be made without ever cloning and expressing the full length protein.

In addition, the suitability of any URS to serve as an antigen or target of a capture agent can be further checked against other available information. For example, since amino acid sequence of many proteins can now be inferred from available genomic data, sequence from the structure of the proteins unique to the sample can be determined by computer aided searching, and the location of the peptide in the protein, and whether it will be accessible in the intact protein, can be determined. Once a suitable URS peptide is found, it can be synthesized using known techniques. With a sample of the URS in hand, an agent that interacts with the peptide such as an antibody or peptidic binder, can be raised against it or panned from a library. In this situation, care must be taken to assure that any chosen fragmentation protocol for the sample does not restrict the protein in a way that destroys or masks the URS. This can be determined theoretically and/or experimentally, and the process can be repeated until the selected URS is reliably retrieved by a capture agent(s).

The URS set selected according to the teachings of the present invention can be used to generate peptides either through enzymatic cleavage of the protein from which they were generated and selection of peptides, or preferably through peptide synthesis methods.

Proteolytically cleaved peptides can be separated by chromatographic or electrophoretic procedures and purified and renatured via well known prior art methods.

Synthetic peptides can be prepared by classical methods known in the art, for example, by using standard solid phase techniques. The standard methods include exclusive solid phase synthesis, partial solid phase synthesis methods, fragment condensation, classical solution synthesis, and even by recombinant DNA technology. See, e.g., Merrifield, J. Am. Chem. Soc., 85:2149 (1963), incorporated herein by reference. Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. W H Freeman and Co. N.Y.] and the composition of which can be confirmed via amino acid sequencing.

In addition, other additives such as stabilizers, buffers, blockers and the like may also be provided with the capture agent.

A. Antibodies

In one embodiment, the capture agent is an antibody or an antibody-like molecule (collectively “antibody”). Thus an antibody useful as capture agent may be a full length antibody or a fragment thereof, which includes an “antigen-binding portion” of an antibody. The term “antigen-binding portion,” as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546 ), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any V_{H} and V_{L} sequences of specific scFv can be linked to human immunoglobulin constant region cDNA or genomic sequences, in order to generate expression vectors encoding complete IgG molecules or other isotypes. V_{H} and V_{L} can also be used in the generation of Fab , Fv or other fragments of immunoglobulins using either protein chemistry or recombinant DNA technology. Other forms of single chain antibodies, such as diabodies are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques.

Antibodies may be polyclonal or monoclonal. The terms “monoclonal antibodies” and “monoclonal antibody composition,” as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody composition, typically displays a single binding affinity for a particular antigen with which it immunoreacts.

Any art-recognized methods can be used to generate an URS-directed antibody. For example, a URS (alone or linked to a hapten) can be used to immunize a suitable subject, (e.g., rabbit, goat, mouse or other mammal or vertebrate). For example, the methods described in U.S. Pat. Nos. 5,422,110; 5,837,268; 5,708,155; 5,723,129; and 5,849,531 (the contents of each of which are incorporated herein by reference) can be used. The immunogenic preparation can further include an adjuvant, such as Freund's complete or incomplete adjuvant, or similar immunostimulatory agent. Immunization of a suitable subject with a URS induces a polyclonal anti-URS antibody response. The antiURS antibody titer in the immunized subject can be monitored over time by standard techniques, such as with an enzyme linked immunosorbent assay (ELISA) using immobilized URS.

The antibody molecules directed against a URS can be isolated from the mammal (e.g., from the blood) and further purified by well known techniques, such as protein A chromatography to obtain the IgG fraction. At an appropriate time after immunization, e.g., when the anti-URS antibody titers are highest, antibody-producing cells can be obtained from the subject and used to prepare, e.g., monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), or the EBV-hybridoma technique (Cole et al. (1985), Monoclonal Antibodies and Cancer Therapy, Alan R. Liss, Inc., pp. 77-96). The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a URS immunogen as described above, and the culture supernatants of the resulting hybridoma cells are screened to identify a hybridoma producing a monoclonal antibody that binds a URS.

Any of the many well known protocols used for fusing lymphocytes and immortalized cell lines can be applied for the purpose of generating an anti-URS monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine-(“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a URS, e.g., using a standard ELISA assay.

In addition, automated screening of antibody or scaffold libraries against arrays of target proteins/URSs will be the most rapid way of developing thousands of reagents that can be used for protein expression profiling. Furthermore, polyclonal antisera, hybridomas or selection from library systems may also be used to quickly generate the necessary capture aganets. A high-throughput process for antibody isolation is described by Hayhurst and Georgiou in Curr Opin Chem Biol 5(6):683-9, December 2001 (incorporated by reference).

B. Proteins and Peptides

Other methods for generating the capture agents of the present invention include phage-display technology described in, for example, Dower et al., WO 91/17271, McCafferty et al., WO 92/01047, Herzig et al., U.S. Pat. No. 5,877,218, Winter et al., U.S. Pat. No. 5,871,907, Winter et al., U.S. Pat. No. 5,858,657, Holliger et al., U.S. Pat. No. 5,837,242, Johnson et al., U.S. Pat. No. 5,733,743 and Hoogenboom et al., U.S. Pat. No. 5,565,332 (the contents of each of which are incorporated by reference). In these methods, libraries of phage are produced in which members display different antibodies, antibody binding sites, or peptides on their outer surfaces. Antibodies are usually displayed as Fv or Fab fragments. Phage displaying sequences with a desired specificity are selected by affinity enrichment to a specific URS.

Methods such as yeast display and in vitro ribosome display may also be used to generate the capture agents of the present invention. The foregoing methods are described in, for example, Methods in Enzymology Vol 328-Part C: Protein-protein interactions & Genomics and Bradbury A. (2001) Nature Biotechnology 19:528-529, the contents of each of which are incorporated herein by reference.

In a related embodiment, proteins or polypeptides may also act as capture agents of the present invention. These peptide capture agents also specifically bind to an given URS, and can be identified, for example, using phage display screening against an immobilized URS, or using any other art-recognized methods. Once identified, the peptidic capture agents may be prepared by any of the well known methods for preparing peptidic sequences. For example, the peptidic capture agents may be produced in prokaryotic or eukaryotic host cells by expression of polynucleotides encoding the particular peptide sequence. Alternatively, such peptidic capture agents may be synthesized by chemical methods. Methods for expression of heterologous peptides in recombinant hosts, chemical synthesis of peptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken, I. M. (1981) CRC Crit. Rev. Biochem. 11:255; Kaiser et al. (1989) Science 243:187; Merrifield, B. (1986) Science 232:342; Kent, S. B. H. (1988) Ann. Rev. Biochem. 57:957; and Offord, R. E. (1980) Semisynthetic Proteins, Wiley Publishing, which are incorporated herein in their entirety by reference).

The peptidic capture agents may also be prepared by any suitable method for chemical peptide synthesis, including solution-phase and solid-phase chemical synthesis. Preferably, the peptides are synthesized on a solid support. Methods for chemically synthesizing peptides are well known in the art (see, e.g., Bodansky, M. Principles of Peptide Synthesis, Springer Verlag, Berlin (1993) and Grant, G. A (ed.). Synthetic Peptides: A User's Guide, W. H. Freeman and Company, New York (1992). Automated peptide synthesizers useful to make the peptidic capture agents are commercially available.

C. Scaffolded Peptides

An alternative approach to generating capture agents for use in the present invention makes use of antibodies are scaffolded peptides, e.g., peptides displayed on the surface of a protein. The idea is that restricting the degrees of freedom of a peptide by incorporating it into a surface-exposed protein loop could reduce the entropic cost of binding to a target protein, resulting in higher affinity. Thioredoxin, fibronectin, avian pancreatic polypeptide (aPP) and albumin, as examples, are small, stable proteins with surface loops that will tolerate a great deal of sequence variation. To identify scaffolded peptides that selectively bind a target URS, libraries of chimeric proteins can be generated in which random peptides are used to replace the native loop sequence, and through a process of affinity maturation, those which selectively bind a URS of interest are identified.

D. Simple Peptides and Peptidomimetic Compounds

Peptides are also attractive candidates for capture agents because they combine advantages of small molecules and proteins. Large, diverse libraries can be made either biologically or synthetically, and the “hits” obtained in binding screens against URS moieties can be made synthetically in large quantities.

Peptide-like oligomers (Soth et al. (1997) Curr. Opin. Chem. Biol. 1:120-129) such as peptoids (Figliozzi et al., (1996) Methods Enzymol. 267:437-447) can also be used as capture reagents, and can have certain advantages over peptides. They are impervious to proteases and their synthesis can be simpler and cheaper than that of peptides, particularly if one considers the use of functionality that is not found in the 20 common amino acids.

E. Nucleic Acids

In another embodiment, aptamers binding specifically to a URS may also be used as capture agents. As used herein, the term “aptamer,” e.g., RNA aptamer or DNA aptamer, includes single-stranded oligonucleotides that bind specifically to a target molecule. Aptamers are selected, for example, by employing an in vitro evolution protocol called systematic evolution of ligands by exponential enrichment. Aptamers bind tightly and specifically to target molecules; most aptamers to proteins bind with a K_{d} (equilibrium dissociation constant) in the range of 1 pM to 1 nM. Aptamers and methods of preparing them are described in, for example, E. N. Brody et al. (1999) Mol. Diagn. 4:381-388, the contents of which are incorporated herein by reference.

In one embodiment, the subject aptamers can be generated using SELEX, a method for generating very high affinity receptors that are composed of nucleic acids instead of proteins. See, for example,. Brody et al. (1999) Mol. Diagn. 4:381-388. SELEX offers a completely in vitro combinatorial chemistry alternative to traditional protein-based antibody technology. Similar to phage display, SELEX is advantageous in terms of obviating animal hosts, reducing production time and labor, and simplifying purification involved in generating specific binding agents to a particular target URS.

To further illustrate, SELEX can be performed by synthesizing a random oligonucleotide library, e.g., of greater than 20 bases in length, which is flanked by known primer sequences. Synthesis of the random region can be achieved by mixing all four nucleotides at each position in the sequence. Thus, the diversity of the random sequence is maximally 4ⁿ, where n is the length of the sequence, minus the frequency of palindromes and symmetric sequences. The greater degree of diversity conferred by SELEX affords greater opportunity to select for oligonuclotides that form 3-dimensional binding sites. Selection of high affinity oligonucleotides is achieved by exposing a random SELEX library to an immobilized target URS. Sequences, which bind readily without washing away, are retained and amplified by the PCR, for subsequent rounds of SELEX consisting of alternating affinity selection and PCR amplification of bound nucleic acid sequences. Four to five rounds of SELEX are typically sufficient to produce a high affinity set of aptamers.

Therefore, hundreds to thousands of aptamers can be made in an economically feasible fashion. Blood and urine can be analyzed on aptamer chips that capture and quantitate proteins. SELEX has also been adapted to the use of 5-bromo (5-Br) and 5-iodo (5-I) deoxyuridine residues. These halogenated bases can be specifically cross-linked to proteins. Selection pressure during in vitro evolution can be applied for both binding specificity and specific photo-cross-linkability. These are sufficiently independent parameters to allow one reagent, a photo-cross-linkable aptamer, to substitute for two reagents, the capture antibody and the detection antibody, in a typical sandwich array. After a cycle of binding, washing, cross-linking, and detergent washing, proteins will be specifically and covalently linked to their cognate aptamers. Because no other proteins are present on the chips, protein-specific stain will now show a meaningful array of pixels on the chip. Combined with learning algorithms and retrospective studies, this technique should lead to a robust yet simple diagnostic chip.

In yet another related embodiment, a capture agent may be an allosteric ribozyme. The term “allosteric ribozymes,” as used herein, includes single-stranded oligonucleotides that perform catalysis when triggered with a variety of effectors, e.g., nucleotides, second messengers, enzyme cofactors, pharmaceutical agents, proteins, and oligonucleotides. Allosteric ribozymes and methods for preparing them are described in, for example, S. Seetharaman et al. (2001) Nature Biotechnol. 19: 336-341, the contents of which are incorporated herein by reference. According to Seetharaman et al., a prototype biosensor array has been assembled from engineered RNA molecular switches that undergo ribozyme-mediated self-cleavage when triggered by specific effectors. Each type of switch is prepared with a 5′-thiotriphosphate moiety that permits immobilization on gold to form individually addressable pixels. The ribozymes comprising each pixel become active only when presented with their corresponding effector, such that each type of switch serves as a specific analyte sensor. An addressed array created with seven different RNA switches was used to report the status of targets in complex mixtures containing metal ion, enzyme cofactor, metabolite, and drug analytes. The RNA switch array also was used to determine the phenotypes of Escherichia coli strains for adenylate cyclase function by detecting naturally produced 3′,5′-cyclic adenosine monophosphate (cAMP) in bacterial culture media.

F. Plastibodies

In certain embodiments the subject capture agent is a plastibody. The term “plastibody” refers to polymers imprinted with selected template molecules. See, for example, Bruggemann (2002) Adv Biochem Eng Biotechnol 76:127-63; and Haupt et al. (1998) Trends Biotech. 16:468-475. The plastibody principle is based on molecular imprinting, namely, a recognition site that can be generated by stereoregular display of pendant functional groups that are grafted to the sidechains of a polymeric chain to thereby mimic the binding site of, for example, an antibody.

G. Chimeric Binding Agents Derived from Two Low-Affinity Ligands

Still another strategy for generating suitable capture agents is to link two or more modest-affinity ligands and generate high affinity capture agent. Given the appropriate linker, such chimeric compounds can exhibit affinities that approach the product of the affinities for the two individual ligands for the URS. To illustrate, a collection of compounds is screened at high concentrations for weak interactors of a target URS. The compounds that do not compete with one another are then identified and a library of chimeric compounds is made with linkers of different length. This library is then screened for binding to the URS at much lower concentrations to identify high affinity binders. Such a technique may also be applied to peptides or any other type of modest-affinity URS-binding compound.

H. Labels for Capture Agents

The capture agents of the present invention may be modified to enable detection using techniques known to one of ordinary skill in the art, such as fluorescent, radioactive, chromatic, optical, and other physical or chemical labels, as described herein below.

I. Miscellaneous

In addition, for any given URS, multiple capture agents belonging to each of the above described categories of capture agents may be available. These multiple capture agents may have different properties, such as affinity/avidity/specificity for the URS. Different affinities are useful in covering the wide dynamic ranges of expression which some proteins can exhibit. Depending on specific use, in any given array of capture agents, different types/amounts of capture agents may be present on a single chip/array to achieve optimal overall performance.

In a preferred embodiment, capture agents are raised against URSs that are located on the surface of the protein of interest, e.g., hydrophilic regions. URSs that are located on the surface of the protein of interest may be identified using any of the well known software available in the art. For example, the Naccess program may be used.

Naccess is a program that calculates the accessible area of a molecule from a PDB (Protein Data Bank) format file. It can calculate the atomic and residue accessiblities for both proteins and nucleic acids. Naccess calculates the atomic accessible area when a probe is rolled around the Van der Waal's surface of a macromolecule. Such three-dimensional co-ordinate sets are available from the PDB at the Brookhaven National laboratory. The program uses the Lee & Richards (1971) J. Mol. Biol., 55, 379-400 method, whereby a probe of given radius is rolled around the surface of the molecule, and the path traced out by its center is the accessible surface.

The solvent accessibility method described in Boger, J., Emini, E. A. & Schmidt, A., Surface probability profile-An heuristic approach to the selection of synthetic peptide antigens, Reports on the Sixth International Congress in Immunology (Toronto) 1986 p.250 also may be used to identify URSs that are located on the surface of the protein of interest. The package MOLMOL (Koradi, R. et al. (1996) J. MoL Graph. 14:51-55) and Eisenhaber's ASC method (Eisenhaber and Argos (1993) J. Comput. Chem. 14:1272-1280; Eisenhaber et al. (1995) J. Comput. Chem. 16:273-284) may also be used.

In another embodiment, capture agents are raised that are designed to bind with peptides generated by digestion of intact proteins rather than with accessible peptidic surface regions on the proteins. In this embodiment, it is preferred to employ a fragmentation protocol which reproducibly generates all of the URSs in the sample under study.

II. Tools Comprising Capture Agents (Arrays, etc.)

In certain embodiments, to construct arrays, e.g., high-density arrays, of capture agents for efficient screening of complex chemical or biological samples or large numbers of compounds, the capture agents need to be immobilized onto a solid support (e.g., a planar support or a bead). A variety of methods are known in the art for attaching biological molecules to solid supports. See, generally, Affinity Techniques, Enzyme Purification: Part B, Meth. Enz. 34 (ed. W. B. Jakoby and M. Wilchek, Acad. Press, N.Y. 1974) and Immobilized Biochemicals and Affinity Chromatography, Adv. Exp. Med. Biol. 42 (ed. R. Dunlap, Plenum Press, N.Y. 1974). The following are a few considerations when constructing arrays.

A. Formats and Surfaces Consideration

Protein arrays have been designed as a miniaturisation of familiar immunoassay methods such as ELISA and dot blotting, often utilising fluorescent readout, and facilitated by robotics and high throughput detection systems to enable multiple assays to be carried out in parallel. Common physical supports include glass slides, silicon, microwells, nitrocellulose or PVDF membranes, and magnetic and other microbeads. While microdrops of protein delivered onto planar surfaces are widely used, related alternative architectures include CD centrifugation devices based on developments in microfluidics [Gyros] and specialised chip designs, such as engineered microchannels in a plate [The Living Chip™, Biotrove] and tiny 3D posts on a silicon surface [Zyomyx]. Particles in suspension can also be used as the basis of arrays, providing they are coded for identification; systems include colour coding for microbeads [Luminex, Bio-Rad] and semiconductor nanocrystals [QDots™, Quantum Dots], and barcoding for beads [UltraPlex™, Smartbeads] and multimetal microrods [Nanobarcodes™ particles, Surromed]. Beads can also be assembled into planar arrays on semiconductor chips [LEAPS technology, BioArray Solutions].

B. Immobilisation Considerations

The variables in immobilisation of proteins such as antibodies include both the coupling reagent and the nature of the surface being coupled to. Ideally, the immobilisation method used should be reproducible, applicable to proteins of different properties (size, hydrophilic, hydrophobic), amenable to high throughput and automation, and compatible with retention of fully functional protein activity. Orientation of the surface-bound protein is recognised as an important factor in presenting it to ligand or substrate in an active state; for capture arrays the most efficient binding results are obtained with orientated capture reagents, which generally requires site-specific labelling of the protein.

The properties of a good protein array support surface are that it should be chemically stable before and after the coupling procedures, allow good spot morphology, display minimal nonspecific binding, not contribute a background in detection systems, and be compatible with different detection systems.

Both covalent and noncovalent methods of protein immobilisation are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatisation may alter the function of the protein and requires a stable interactive surface. Biological capture methods utilising a tag on the protein provide a stable linkage and bind the protein specifically and in reproducible orientation, but the biological reagent must first be immobilised adequately and the array may require special handling and have variable stability.

Several immobilisation chemistries and tags have been described for fabrication of protein arrays. Substrates for covalent attachment include glass slides coated with aminoor aldehyde-containing silane reagents [Telechem]. In the Versalinx™ system [Prolinx], reversible covalent coupling is achieved by interaction between the protein derivatised with phenyldiboronic acid, and salicylhydroxamic acid immobilised on the support surface. This also has low background binding and low intrinsic fluorescence and allows the immobilised proteins to retain function. Noncovalent binding of unmodified protein occurs within porous structures such as HydroGel™ [PerkinElmer], based on a 3-dimensional polyacrylamide gel; this substrate is reported to give a particularly low background on glass microarrays, with a high capacity and retention of protein function. Widely used biological capture methods are through biotin/streptavidin or hexahistidine/Ni interactions, having modified the protein appropriately. Biotin may be conjugated to a poly-lysine backbone immobilised on a surface such as titanium dioxide [Zyomyx] or tantalum pentoxide [Zeptosens].

Arenkov et al., for example, have described a way to immobilize proteins while preserving their function by using microfabricated polyacrylamide gel pads to capture proteins, and then accelerating diffusion through the matrix by microelectrophoresis (Arenkov et al. (2000), Anal Biochem 278(2):123-31). The patent literature also describes a number of different methods for attaching biological molecules to solid supports. For example, U.S. Pat. No. 4,282,287 describes a method for modifying a polymer surface through the successive application of multiple layers of biotin, avidin, and extenders. U.S. Pat. No. 4,562,157 describes a technique for attaching biochemical ligands to surfaces by attachment to a photochemically reactive arylazide. U.S. Pat. No. 4,681,870 describes a method for introducing free amino or carboxyl groups onto a silica matrix, in which the groups may subsequently be covalently linked to a protein in the presence of a carbodiimide. In addition, U.S. Pat. No. 4,762,881 describes a method for attaching a polypeptide chain to a solid substrate by incorporating a light-sensitive unnatural amino acid group into the polypeptide chain and exposing the product to low-energy ultraviolet light.

The surface of the support is chosen to possess, or is chemically derivatized to possess, at least one reactive chemical group that can be used for further attachment chemistry. There may be optional flexible adapter molecules interposed between the support and the capture agents. In one embodiment, the capture agents are physically adsorbed onto the support.

In certain embodiments of the invention, a capture agent is immobilized on a support in ways that separate the capture agent's URS binding site region and the region where it is linked to the support. In a preferred embodiment, the capture agent is engineered to form a covalent bond between one of its termini to an adapter molecule on the support. Such a covalent bond may be formed through a Schiff-base linkage, a linkage generated by a Michael addition, or a thioether linkage.

In order to allow attachment by an adapter or directly by a capture agent, the surface of the substrate may require preparation to create suitable reactive groups. Such reactive groups could include simple chemical moieties such as amino, hydroxyl, carboxyl, carboxylate, aldehyde, ester, amide, amine, nitrile, sulfonyl, phosphoryl, or similarly chemically reactive groups. Alternatively, reactive groups may comprise more complex moieties that include, but are not limited to, sulfo-N-hydroxysuccinimide, nitrilotriacetic acid, activated hydroxyl, haloacetyl (e.g., bromoacetyl, iodoacetyl), activated carboxyl, hydrazide, epoxy, aziridine, sulfonylchloride, trifluoromethyldiaziridine, pyridyldisulfide, N-acyl-imidazole, imidazolecarbamate, succinimidylcarbonate, arylazide, anhydride, diazoacetate, benzophenone, isothiocyanate, isocyanate, imidoester, fluorobenzene, biotin and avidin. Techniques of placing such reactive groups on a substrate by mechanical, physical, electrical or chemical means are well known in the art, such as described by U.S. Pat. No. 4,681,870, incorporated herein by reference.

Once the initial preparation of reactive groups on the substrate is completed (if necessary), adapter molecules optionally may be added to the surface of the substrate to make it suitable for further attachment chemistry. Such adapters covalently join the reactive groups already on the substrate and the capture agents to be immobilized, having a backbone of chemical bonds forming a continuous connection between the reactive groups on the substrate and the capture agents, and having a plurality of freely rotating bonds along that backbone. Substrate adapters may be selected from any suitable class of compounds and may comprise polymers or copolymers of organic acids, aldehydes, alcohols, thiols, amines and the like. For example, polymers or copolymers of hydroxy-, amino-, or di-carboxylic acids, such as glycolic acid, lactic acid, sebacic acid, or sarcosine may be employed. Alternatively, polymers or copolymers of saturated or unsaturated hydrocarbons such as ethylene glycol, propylene glycol, saccharides, and the like may be employed. Preferably, the substrate adapter should be of an appropriate length to allow the capture agent, which is to be attached, to interact freely with molecules in a sample solution and to form effective binding. The substrate adapters may be either branched or unbranched, but this and other structural attributes of the adapter should not interfere stereochemically with relevant functions of the capture agents, such as a URS interaction. Protection groups, known to those skilled in the art, may be used to prevent the adapter's end groups from undesired or premature reactions. For instance, U.S. Pat. No. 5,412,087, incorporated herein by reference, describes the use of photo-removable protection groups on a adapter's thiol group.

To preserve the binding affinity of a capture agent, it is preferred that the capture agent be modified so that it binds to the support substrate at a region separate from the region responsible for interacting with it's ligand, i.e., the URS.

Methods of coupling the capture agent to the reactive end groups on the surface of the substrate or on the adapter include reactions that form linkage such as thioether bonds, disulfide bonds, amide bonds, carbamate bonds, urea linkages, ester bonds, carbonate bonds, ether bonds, hydrazone linkages, Schiff-base linkages, and noncovalent linkages mediated by, for example, ionic or hydrophobic interactions. The form of reaction will depend, of course, upon the available reactive groups on both the substrate/adapter and capture agent.

C. Array Fabrication Consideration

Preferably, the immobilized capture agents are arranged in an array on a solid support, such as a silicon-based chip or glass slide. One or more capture agents designed to detect the presence (and optionally the concentration) of a given known protein (one previously recognized as existing) is immobilized at each of a plurality of cells/regions in the array. Thus, a signal at a particular cell/region indicates the presence of a known protein in the sample, and the identity of the protein is revealed by the position of the cell. Alternatively, capture agents for one or a plurality of URS are immobilized on beads, which optionally are labeled to identify their intended target analyte, or are distributed in an array such as a microwell plate.

In one embodiment, the microarray is high density, with a density over about 100, preferably over about 1000, 1500, 2000, 3000, 4000, 5000 and further preferably over about 9000, 10000, 11000, 12000 or 13000 spots per cm², formed by attaching capture agents onto a support surface which has been functionalized to create a high density of reactive groups or which has been functionalized by the addition of a high density of adapters bearing reactive groups. In another embodiment, the microarray comprises a relatively small number of capture agents, e.g., 10 to 50, selected to detect in a sample various combinations of specific proteins which generate patterns probative of disease diagnosis, cell type determination, pathogen identification, etc.

Although the characteristics of the substrate or support may vary depending upon the intended use, the shape, material and surface modification of the substrates must be considered. Although it is preferred that the substrate have at least one surface which is substantially planar or flat, it may also include indentations, protuberances, steps, ridges, terraces and the like and may have any geometric form (e.g., cylindrical, conical, spherical, concave surface, convex surface, string, or a combination of any of these). Suitable substrate materials include, but are not limited to, glasses, ceramics, plastics, metals, alloys, carbon, papers, agarose, silica, quartz, cellulose, polyacrylamide, polyamide, and gelatin, as well as other polymer supports, other solid-material supports, or flexible membrane supports. Polymers that may be used as substrates include, but are not limited to: polystyrene; poly(tetra)fluoroethylene (PTFE); polyvinylidenedifluoride; polycarbonate; polymethylmethacrylate; polyvinylethylene; polyethyleneimine; polyoxymethylene (POM); polyvinylphenol; polylactides; polymethacrylimide (PMI); polyalkenesulfone (PAS); polypropylene; polyethylene; polyhydroxyethylmethacrylate (HEMA); polydimethylsiloxane; polyacrylamide; polyimide; and various block copolymers. The substrate can also comprise a combination of materials, whether water-permeable or not, in multi-layer configurations. A preferred embodiment of the substrate is a plain 2.5 cm×7.5 cm glass slide with surface Si—OH functionalities.

Array fabrication methods include robotic contact printing, ink-jetting, piezoelectric spotting and photolithography. A number of commercial arrayers are available [e.g. Packard Biosience] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

A microfluidics system for automated sample incubation with arrays on glass slides and washing has been codeveloped by NextGen and PerkinElmer Lifesciences.

For example, capture agent microarrays may be produced by a number of means, including “spotting” wherein small amounts of the reactants are dispensed to particular positions on the surface of the substrate. Methods for spotting include, but are not limited to, microfluidics printing, microstamping (see, e.g., U.S. Pat. No. 5,515,131, U.S. Pat. No. 5,731,152, Martin, B. D. et al. (1998), Langmuir 14: 3971-3975 and Haab, B B et al. (2001) Genome Biol 2 and MacBeath, G. et al. (2000) Science 289: 1760 1763), microcontact printing (see, e.g., PCT Publication WO 96/29629), inkjet head printing (Roda, A. et al. (2000) BioTechniques 28: 492-496, and Silzel, J. W. et al. (1998) Clin Chem 44: 2036 2043), microfluidic direct application (Rowe, C. A. et al. (1999) Anal Chem 71: 433-439 and Bernard, A. et al. (2001), Anal Chem 73: 8-12) and electrospray deposition (Morozov, V. N. et al. (1999) Anal Chem 71: 1415-1420 and Moerman R. et al. (2001) Anal Chem 73: 2183-2189). Generally, the dispensing device includes calibrating means for controlling the amount of sample deposition, and may also include a structure for moving and positioning the sample in relation to the support surface. The volume of fluid to be dispensed per capture agent in an array varies with the intended use of the array, and available equipment. Preferably, a volume formed by one dispensation is less than 100 nL, more preferably less than 10 nL, and most preferably about 1 nL. The size of the resultant spots will vary as well, and in preferred embodiments these spots are less than 20,000 μm in diameter, more preferably less than 2,000 μm in diameter, and most preferably about 150-200 μm in diameter (to yield about 1600 spots per square centimeter). Solutions of blocking agents may be applied to the microarrays to prevent non-specific binding by reactive groups that have not bound to a capture agent. Solutions of bovine serum albumin (BSA), casein, or nonfat milk, for example, may be used as blocking agents to reduce background binding in subsequent assays.

In preferred embodiments, high-precision, contact-printing robots are used to pick up small volumes of dissolved capture agents from the wells of a microtiter plate and to repetitively deliver approximately 1 nL of the solutions to defined locations on the surfaces of substrates, such as chemically-derivatized glass microscope slides. Examples of such robots include the GMS 417 Arrayer, commercially available from Affymetrix of Santa Clara, Calif., and a split pin arrayer constructed according to instructions downloadable from the Brown lab website at http://cmgm.stanford.edu/pbrown. This results in the formation of microscopic spots of compounds on the slides. It will be appreciated by one of ordinary skill in the art, however, that the current invention is not limited to the delivery of 1 nL volumes of solution, to the use of particular robotic devices, or to the use of chemically derivatized glass slides, and that alternative means of delivery can be used that are capable of delivering picoliter or smaller volumes. Hence, in addition to a high precision array robot, other means for delivering the compounds can be used, including, but not limited to, ink jet printers, piezoelectric printers, and small volume pipetting robots.

In one embodiment, the compositions, e.g., microarrays or beads, comprising the capture agents of the present invention may also comprise other components, e.g., molecules that recognize and bind specific peptides, metabolites, drugs or drug candidates, RNA, DNA, lipids, and the like. Thus, an array of capture agents only some of which bind a URS can comprise an embodiment of the invention.

As an alternative to planar microarrays, bead-based assays combined with fluorescence-activated cell sorting (FACS) have been developed to perform multiplexed immunoassays. Fluorescence-activated cell sorting has been routinely used in diagnostics for more than 20 years. Using mAbs, cell surface markers are identified on normal and neoplastic cell populations enabling the classification of various forms of leukemia or disease monitoring (recently reviewed by Herzenberg et al. Immunol Today 21 (2000), pp. 383-390).

Bead-based assay systems employ microspheres as solid support for the capture molecules instead of a planar substrate, which is conventionally used for microarray assays. In each individual immunoassay, the capture agent is coupled to a distinct type of microsphere. The reaction takes place on the surface of the microspheres. The individual microspheres are color-coded by a uniform and distinct mixture of red and orange fluorescent dyes. After coupling to the appropriate capture molecule, the different colorcoded bead sets can be pooled and the immunoassay is performed in a single reaction vial. Product formation of the URS targets with their respective capture agents on the different bead types can be detected with a fluorescence-based reporter system. The signal intensities are measured in a flow cytometer, which is able to quantify the amount of captured targets on each individual bead. Each bead type and thus each immobilized target is identified using the color code measured by a second fluorescence signal. This allows the multiplexed quantification of multiple targets from a single sample. Sensitivity, reliability and accuracy are similar to those observed with standard microtiter ELISA procedures. Colour-coded microspheres can be used to perform up to a hundred different assay types simultaneously (LabMAP system, Laboratory Muliple Analyte Profiling, Luminex, Austin, Tex., USA). For example, microsphere-based systems have been used to simultaneously quantify cytokines or autoantibodies from biological samples (Carson and Vignali, J Immunol Methods 227 (1999), pp. 41-5²; Chen et al., Clin Chem 45 (1999), pp. 1693-1694; Fulton et al., Clin Chem 43 (1997), pp. 1749-1756). Bellisario et al. (Early Hum Dev 64 (2001), pp. 21-25) have used this technology to simultaneously measure antibodies to three HIV-1 antigens from newborn dried blood-spot specimens.

Bead-based systems have several advantages. As the capture molecules are coupled to distinct microspheres, each individual coupling event can be perfectly analysed. Thus, only quality-controlled beads can be pooled for multiplexed immunoassays. Furthermore, if an additional parameter has to be included into the assay, one must only add a new type of loaded bead. No washing steps are required when performing the assay. The sample is incubated with the different bead types together with fluorescently labeled detection antibodies. After formation of the sandwich immuno-complex, only the fluorophores that are definitely bound to the surface of the microspheres are counted in the flow cytometer.

D. Related Non-Array Formats

An alternative to an array of capture agents is one made through the so-called “molecular imprinting” technology, in which peptides (e.g. selected URSs) are used as templates to generate structurally complementary, sequence-specific cavities in a polymerisable matrix; the cavities can then specifically capture (digested) proteins which have the appropriate primary amino acid sequence [ProteinPrint™, Aspira Biosystems]. To illustrate, a chosen URS can be synthesized, and a universal matrix of polymerizable monomers is allowed to self assemble around the peptide and crosslinked into place. The URS, or template, is then removed, leaving behind a cavity complementary in shape and functionality. The cavities can be formed on a film, discrete sites of an array or the surface of beads. When a sample of fragmented proteins is exposed to the capture agent, the polymer will selectively retain the target protein containing the URS and exclude all others. After the washing, only the bound URS-containing peptides remain. Common staining and tagging procedures, or any of the non-labeling techniques described below can be used to detect expression levels and/or post translational modifications. Alternatively, the captured peptides can be eluted for further analysis such as mass spectrometry analysis. See WO 01/61354 A1, WO 01/61355 A1, and related applications/patents.

Another methodology which can be used diagnostically and in expression profiling is the ProteinChip® array [Ciphergen], in which solid phase chromatographic surfaces bind proteins with similar characteristics of charge or hydrophobicity from mixtures such as plasma or tumour extracts, and SELDI-TOF mass spectrometry is used to detection the retained proteins. The ProteinChip® is credited with the ability to identify novel disease markers. However, this technology differs from the protein arrays under discussion here since, in general, it does not involve immobilisation of individual proteins for detection of specific ligand interactions.

E. Single Assay Format

URS-specific affinity capture agents can also be used in a single assay format. For example, such agents can be used to develop a better assay for detecting circulating agents, such as PSA, by providing increased sensitivity, dynamic range and/or recovery rate. For instance, the single assays can have functional performance characteristics which exceed traditional ELISA and other immunoassays, such as one or more of the following: a regression coefficient (R2) of 0.95 or greater for a reference standard, e.g., a comparable control sample, more preferably an R2 greater than 0.97, 0.99 or even 0.995; a recovery rate of at least 50 percent, and more preferably at least 60, 75, 80 or even 90 percent; a positive predictive value for occurrence of the protein in a sample of at least 90 percent, more preferably at least 95, 98 or even 99 percent; a diagnostic sensitivity (DSN) for occurrence of the protein in a sample of 99 percent or higher, more preferably at least 99.5 or even 99.8 percent; a diagnostic specificity (DSP) for occurrence of the protein in a sample of 99 percent or higher, more preferably at least 99.5 or even 99.8 percent.

III. Methods of Detecting Binding Events

The capture agents of the invention, as well as compositions, e.g., microarrays or beads, comprising these capture agents have a wide range of applications in the health care industry, e.g., in therapy, in clinical diagnostics, in in vivo imaging or in drug discovery. The capture agents of the present invention also have industrial and environmental applications, e.g., in environmental diagnostics, industrial diagnostics, food safety, toxicology, catalysis of reactions, or high-throughput screening; as well as applications in the agricultural industry and in basic research, e.g., protein sequencing.

The capture agents of the present invention are a powerful analytical tool that enables a user to detect a specific protein, or group of proteins of interest present within complex samples. In addition, the invention allow for efficient and rapid analysis of samples; sample conservation and direct sample comparison. The invention enables “multi-parametric” analysis of protein samples. As used herein, a “multi-parametric” analysis of a protein sample is intended to include an analysis of a protein sample based on a plurality of parameters. For example, a protein sample may be contacted with a plurality of URSs, each of the URSs being able to detect a different protein within the sample. Based on the combination and, preferably the relative concentration, of the proteins detected in the sample the skilled artisan would be able to determine the identity of a sample, diagnose a disease or pre-disposition to a disease, or determine the stage of a disease

The capture agents of the present invention may be used in any method suitable for detection of a protein or a polypeptide, such as, for example, in immunoprecipitations, immunocytochemistry, Western Blots or nuclear magnetic resonance spectroscopy (NMR).

To detect the presence of a protein that interacts with a capture agent, a variety of art known methods may be used. The protein to be detected may be labeled with a detectable label, and the amount of bound label directly measured. The term “label” is used herein in a broad sense to refer to agents that are capable of providing a detectable signal, either directly or through interaction with one or more additional members of a signal producing system. Labels that are directly detectable and may find use in the present invention include, for example, fluorescent labels such as fluorescein, rhodamine, BODIPY, cyanine dyes (e.g. from Amersham Pharmacia), Alexa dyes (e.g. from Molecular Probes, Inc.), fluorescent dye phosphoramidites, beads, chemilumninescent compounds, colloidal particles, and the like. Suitable fluorescent dyes are known in the art, including fluoresceinisothiocyanate (FITC); rhodamine and rhodamine derivatives; Texas Red; phycoerythrin; allophycocyanin; 6-carboxyfluorescein (6-FAM); 2′,7′-dimethoxy-41,51-dichloro carboxyfluorescein (JOE); 6-carboxy-X-rhodamine (ROX); 6-carboxy-21,41,71,4,7-hexachlorofluorescein (HEX); 5-carboxyfluorescein (5-FAM); N,N,N1,N′-tetramethyl carboxyrhodamine (TAMRA); sulfonated rhodamine; Cy3; Cy5, etc. Radioactive isotopes, such as ³⁵S, ³²P, ³H, ¹²⁵I, etc., and the like can also be used for labeling. In addition, labels may also include near-infrared dyes (Wang et al., Anal. Chem., 72:5907-5917 (2000), upconverting phosphors (Hampl et al., Anal. Biochem., 288:176-187 (2001), DNA dendrimers (Stears et al., Physiol. Genomics 3: 93-99 (2000), quantum dots (Bruchez et al., Science 281:2013-2016 (1998), latex beads (Okana et al., Anal. Biochem. 202:120-125 (1992), selenium particles (Stimpson et al., Proc. Natl. Acad. Sci. 92:6379-6383 (1995), and europium nanoparticles (Harma et al., Clin. Chem. 47:561-568 (2001). The label is one that preferably does not provide a variable signal, but instead provides a constant and reproducible signal over a given period of time.

A very useful labeling agent is water-soluable quantum dots, or so-called “functionalized nanocrystals” or “semiconductor nanocrystals”as described in U.S. Pat. No. 6,114,038. Generally, quantum dots can be prepared which result in relative monodispersity (e.g., the diameter of the core varying approximately less than 10% between quantum dots in the preparation), as has been described previously (Bawendi et al., 1993, J. Am. Chem. Soc. 115:8706). Examples of quantum dots are known in the art to have a core selected from the group consisting of CdSe, CdS, and CdTe (collectively referred to as “CdX”)(see, e.g., Norris et al., 1996, Physical Review B. 53:16338-16346; Nirmal et al., 1996, Nature 383:802-804; Empedocles et al., 1996, Physical Review Letters 77:3873-3876; Murray et al., 1996, Science 270: 1355-1338; Effros et al., 1996, Physical Review B. 54:4843-4856; Sacra et al., 1996, J. Chem. Phys. 103:5236-5245; Murakoshi et al., 1998, J. Colloid Interface Sci. 203:225-228; Optical Materials and Engineering News, 1995, Vol. 5, No. 12; and Murray et al., 1993, J. Am. Chem. Soc. 115:8706-8714; the disclosures of which are hereby incorporated by reference).

CdX quantum dots have been passivated with an inorganic coating (“shell”) uniformly deposited thereon. Passivating the surface of the core quantum dot can result in an increase in the quantum yield of the luminescence emission, depending on the nature of the inorganic coating. The shell which is used to passivate the quantum dot is preferably comprised of YZ wherein Y is Cd or Zn, and Z is S, or Se. Quantum dots having a CdX core and a YZ shell have been described in the art (see, e.g., Danek et al., 1996, Chem. Mater. 8:173-179; Dabbousi et al., 1997, J. Phys. Chem. B 101:9463; Rodriguez-Viejo et al., 1997, Appl. Phys. Lett. 70:2132-2134; Peng et al., 1997, J. Am. Chem. Soc. 119:7019-7029; 1996, Phys. Review B. 53:16338-16346; the disclosures of which are hereby incorporated by reference). However, the above described quantum dots, passivated using an inorganic shell, have only been soluble in organic, non-polar (or weakly polar) solvents. To make quantum dots useful in biological applications, it is desirable that the quantum dots are water-soluble. “Water-soluble” is used herein to mean sufficiently soluble or suspendable in an aqueous-based solution, such as in water or water-based solutions or buffer solutions, including those used in biological or molecular detection systems as known by those skilled in the art.

U.S. Pat. No. 6,114,038 provides a composition comprising functionalized nanocrystals for use in non-isotopic detection systems. The composition comprises quantum dots (capped with a layer of a capping compound) that are water-soluble and functionalized by operably linking, in a successive manner, one or more additional compounds. In a preferred embodiment, the one or more additional compounds form successive layers over the nanocrystal. More particularly, the functionalized nanocrystals comprise quantum dots capped with the capping compound, and have at least a diaminocarboxylic acid which is operatively linked to the capping compound. Thus, the functionalized nanocrystals may have a first layer comprising the capping compound, and a second layer comprising a diaminocarboxylic acid; and may further comprise one or more successive layers including a layer of amino acid, a layer of affinity ligand, or multiple layers comprising a combination thereof. The composition comprises a class of quantum dots that can be excited with a single wavelength of light resulting in detectable luminescence emissions of high quantum yield and with discrete luminescence peaks. Such functionalized nanocrystal may be used to label capture agents of the instant invention for their use in the detection and/or quantitation of the binding events.

U.S. Pat. No. 6,326,144 describes quantum dots (QDs) having a characteristic spectral emission, which is tunable to a desired energy by selection of the particle size of the quantum dot. For example, a 2 nanometer quantum dot emits green light, while a 5 nanometer quantum dot emits red light. The emission spectra of quantum dots have linewidths as narrow as 25-30 nm depending on the size heterogeneity of the sample, and lineshapes that are symmetric, gaussian or nearly gaussian with an absence of a tailing region. The combination of tunability, narrow linewidths, and symmetric emission spectra without a tailing region provides for high resolution of multiply-sized quantum dots within a system and enables researchers to examine simultaneously a variety of biological moieties tagged with QDs. In addition, the range of excitation wavelengths of the nanocrystal quantum dots is broad and can be higher in energy than the emission wavelengths of all available quantum dots. Consequently, this allows the simultaneous excitation of all quantum dots in a system with a single light source, usually in the ultraviolet or blue region of the spectrum. QDs are also more robust than conventional organic fluorescent dyes and are more resistant to photobleaching than the organic dyes. The robustness of the QD also alleviates the problem of contamination of the degradation products of the organic dyes in the system being examined. These QDs can be used for labeling capture agents of protein, nucleic acid, and other biological molecules in nature. Cadmium Selenide quantum dot nanocrystals are available from Quantum Dot Corporation of Hayward, Calif.

Alternatively, the sample to be tested is not labeled, but a second stage labeled reagent is added in order to detect the presence or quantitate the amount of protein in the sample. Such “sandwich based” methods of detection have the disadvantage that two capture agents must be developed for each protein, one to capture the URS and one to label it once captured. Such methods have the advantage that they are characterized by an inherently improved signal to noise ratio as they exploit two binding reactions at different points on a peptide, thus the presence and/or concentration of the protein can be measured with more accuracy and precision because of the increased signal to noise ratio.

In yet another embodiment, the subject capture array can be a “virtual arrays”. For example, a virtual array can be generated in which antibodies or other capture agents are immobilized on beads whose identity, with respect to the particular URS it is specific for as a consequence to the associated capture agent, is encoded by a particular ratio of two or more covalently attached dyes. Mixtures of encoded URS-beads are added to a sample, resulting in capture of the URS entities recognized by the immobilized capture agents.

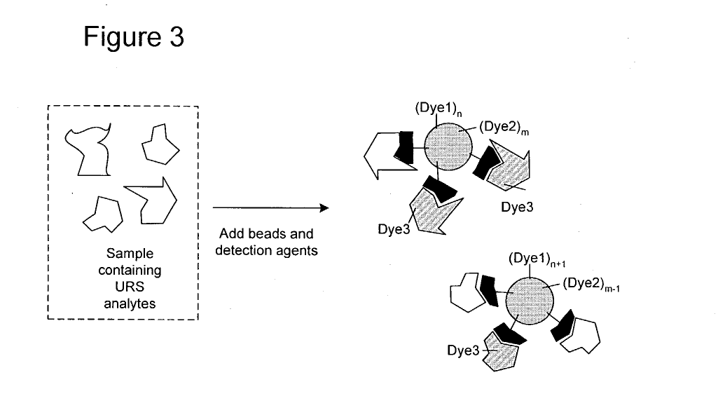

To quantitate the captured species, a sandwich assay with fluorescently labeled antibodies that bind the captured URS, or a competitive binding assay with a fluorescently labeled ligand for the capture agent, are added to the mix. In one embodiment, the labeled ligand is a labeled URS that competes with the analyte URS for binding to the capture agent. The beads are then introduced into an instrument, such as a flow cytometer, that reads the intensity of the various fluorescence signals on each bead, and the identity of the bead can be determined by measuring the ratio of the dyes (FIG. 3). This technology is relatively fast and efficient, and can be adapted by researchers to monitor almost any set of URS of interest.

In another embodiment, an array of capture agents are embedded in a matrix suitable for ionization (such as described in Fung et al. (2001) Curr. Opin. Biotechnol. 12:65-69). After application of the sample and removal of unbound molecules (by washing), the retained URS proteins are analyzed by mass spectrometry. In some instances, further proteolytic digestion of the bound species with trypsin may be required before ionization, particularly if electrospray is the means for ionizing the peptides.

All the above named reagents may be used to label the capture agents. Preferably, the capture agent to be labeled is combined with an activated dye that reacts with a group present on the protein to be detected, e.g., amine groups, thiol groups, or aldehyde groups.

The label may also be a covalently bound enzyme capable of providing a detectable product signal after addition of suitable substrate. Examples of suitable enzymes for use in the present invention include horseradish peroxidase, alkaline phosphatase, malate dehydrogenase and the like.

Enzyme-Linked Immunosorbent Assay (ELISA) may also be used for detection of a protein that interacts with a capture agent. In an ELISA, the indicator molecule is covalently coupled to an enzyme and may be quantified by determining with a spectrophotometer the initial rate at which the enzyme converts a clear substrate to a correlated product. Methods for performing ELISA are well known in the art and described in, for example, Perlmann, H. and Perlmann, P. (1994). Enzyme-Linked Immunosorbent Assay. In: Cell Biology: A Laboratory Handbook. San Diego, Calif., Academic Press, Inc., 322-328; Crowther, J. R. (1995). Methods in Molecular Biology, Vol. 42-ELISA: Theory and Practice. Humana Press, Totowa, N.J.; and Harlow, E. and Lane, D. (1988). Antibodies: A Laboratory Manual. Cold Spring Harbor Laboratory Press, Cold Spring Harbor, N.Y., 553-612, the contents of each of which are incorporated by reference. Sandwich (capture) ELISA may also be used to detect a protein that interacts with two capture agents. The two capture agents may be able to specifically interact with two URSs that are present on the same peptide (e.g., the peptide which has been generated by fragmentation of the sample of interest, as described above). Alternatively, the two capture agents may be able to specifically interact with one URS and one non-unique amino acid sequence, both present on the same peptide (e.g., the peptide which has been generated by fragmentation of the sample of interest, as described above). Sandwich ELISAs for the quantitation of proteins of interest are especially valuable when the concentration of the protein in the sample is low and/or the protein of interest is present in a sample that contains high concentrations of contaminating proteins.

A fully-automated, microarray-based approach for high-throughput, ELISAs was described by Mendoza et al. (BioTechniques 27:778-780,782-786,788, 1999). This system consisted of an optically flat glass plate with 96 wells separated by a Teflon mask. More than a hundred capture molecules were immobilised in each well. Sample incubation, washing and fluorescence-based detection were performed with an automated liquid pipettor. The microarrays were quantitatively imaged with a scanning charge-coupled device (CCD) detector. Thus, the feasibility of multiplex detection of arrayed antigens in a high-throughput fashion using marker antigens could be successfully demonstrated. In addition, Silzel et al. (Clin Chem 44 pp. 2036-2043, 1998) could demonstrate that multiple IgG subclasses can be detected simultaneously using microarray technology. Wiese et al. (Clin Chem 47 pp. 1451-1457, 2001) were able to measure prostate-specific antigen (PSA), -(1)-antichymotrypsin-bound PSA and interleukin-6 in a microarray format. Arenkov et al. (supra) carried out microarray sandwich immunoassays and direct antigen or antibody detection experiments using a modified polyacrylamide gel as substrate for immobilised capture molecules.

Most of the microarray assay formats described in the art rely on chemiluminescence- or fluorescence-based detection methods. A further improvement with regard to sensitivity involves the application of fluorescent labels and waveguide technology. A fluorescence-based array immunosensor was developed by Rowe et al. (Anal Chem 71 (1999), pp. 433439; and Biosens Bioelectron 15 (2000), pp. 579-589) and applied for the simultaneous detection of clinical analytes using the sandwich immunoassay format. Biotinylated capture antibodies were immobilised on avidin-coated waveguides using a flow-chamber module system. Discrete regions of capture molecules were vertically arranged on the surface of the waveguide. Samples of interest were incubated to allow the targets to bind to their capture molecules. Captured targets were then visualised with appropriate fluorescently labelled detection molecules. This array immunosensor was shown to be appropriate for the detection and measurement of targets at physiologically relevant concentrations in a variety of clinical samples.

A further increase in the sensitivity using waveguide technology was achieved with the development of the planar waveguide technology (Duveneck et al., Sens Actuators B B38 (1997), pp. 88-95). Thin-film waveguides are generated from a high-refractive material such as Ta₂O₅ that is deposited on a transparent substrate. Laser light of desired wavelength is coupled to the planar waveguide by means of diffractive grating. The light propagates in the planar waveguide and an area of more than a square centimeter can be homogeneously illuminated. At the surface, the propagating light generates a so-called evanescent field. This extends into the solution and activates only fluorophores that are bound to the surface. Fluorophores in the surrounding solution are not excited. Close to the surface, the excitation field intensities can be a hundred times higher than those achieved with standard confocal excitation. A CCD camera is used to identify signals simultaneously across the entire area of the planar waveguide. Thus, the immobilisation of the capture molecules in a microarray format on the planar waveguide allows the performance of highly sensitive miniaturised and parallelised immunoassays. This system as successfully employed to detect interleukin-6 at concentrations as low as 40 fM and as the additional advantage that the assay can be performed without washing steps that are usually required to remove unbound detection molecules (Weinberger et al., Pharmacogenomics 1 (2000), pp. 395-416).

Alternative strategies pursued to increase sensitivity are based on signal amplification procedures. For example, immunoRCA (immuno rolling circle amplification) involves an oligonucleotide primer that is covalently attached to a detection molecule (such as a second capture agent in a sanwitch-type assay format). Using circular DNA as template, which is complementary to the attached oligonucleotide, DNA polymerase will extend the attached oligonucleotide and generate a long DNA molecule consisting of hundreds of copies of the circular DNA, which remains attached to the detection molecule. The incorporation of thousands of fluorescently labelled nucleotides will generate a strong signal. Schweitzer et al. (Proc Natl Acad Sci USA 97 (2000), pp. 10113-10119) have evaluated this detection technology for use in microarray-based assays. Sandwich immunoassays for hulgE and prostate-specific antigens were performed in a microarray format. The antigens could be detected at femtomolar concentrations and it was possible to score single, specifically captured antigens by counting discrete fluorescent signals that arose from the individual antibody-antigen complexes. The authors demonstrated that immunoassays employing rolling circle DNA amplification are a versatile platform for the ultra-sensitive detection of antigens and thus are well suited for use in protein microarray technology.

Radioimmunoassays (RIA) may also be used for detection of a protein that interacts with a capture agent. In a RIA, the indicator molecule is labeled with a radioisotope and it may be quantified by counting radioactive decay events in a scintillation counter. Methods for performing direct or competitive RIA are well known in the art and described in, for example, Cell Biology: A Laboratory Handbook. San Diego, Calif., Academic Press, Inc., the contents of which are incorporated herein by reference.

Other immunoassays commonly used to quantitate the levels of proteins in cell samples, and are well-known in the art, can be adapted for use in the instant invention. The invention is not limited to a particular assay procedure, and therefore is intended to include both homogeneous and heterogeneous procedures. Exemplary other immunoassays which can be conducted according to the invention include fluorescence polarization immunoassay (FPIA), fluorescence immunoassay (FIA), enzyme immunoassay (EIA), nephelometric inhibition immunoassay (NIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art. In one embodiment, the determination of protein level in a biological sample may be performed by a microarray analysis (protein chip).

In several other embodiments, detection of the presence of a protein that interacts with a capture agent may be achieved without labeling. For example, determining the ability of a protein to bind to a capture agent can be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore).

In another embodiment, a biosensor with a special diffractive grating surface may be used to detect/quantitate binding between non-labeled URS-containing peptides in a treated (digested) biological sample and immobilized capture agents at the surface of the biosensor. Details of the technology is described in more detail in B. Cunningham, P. Li, B. Lin, J. Pepper, “Colorimetric resonant reflection as a direct biochemical assay technique,” Sensors and Actuators B, Volume 81, p. 316-328, Jan. 5 2002, and in PCT No. WO 02/061429 A2 and US 2003/0032039. Briefly, a guided mode resonant phenomenon is used to produce an optical structure that, when illuminated with collimated white light, is designed to reflect only a single wavelength (color). When molecules are attached to the surface of the biosensor, the reflected wavelength (color) is shifted due to the change of the optical path of light that is coupled into the grating. By linking receptor molecules to the grating surface, complementary binding molecules can be detected/quantitated without the use of any kind of fluorescent probe or particle label. The spectral shifts may be analyzed to determine the expression data provided, and to indicate the presence or absence of a particular indication.

The biosensor typically comprises: a two-dimensional grating comprised of a material having a high refractive index, a substrate layer that supports the two-dimensional grating, and one or more detection probes immobilized on the surface of the two-dimensional grating opposite of the substrate layer. When the biosensor is illuminated a resonant grating effect is produced on the reflected radiation spectrum. The depth and period of the two-dimensional grating are less than the wavelength of the resonant grating effect.

A narrow band of optical wavelengths can be reflected from the biosensor when it is illuminated with a broad band of optical wavelengths. The substrate can comprise glass, plastic or epoxy. The two-dimensional grating can comprise a material selected from the group consisting of zinc sulfide, titanium dioxide, tantalum oxide, and silicon nitride.

The substrate and two-dimensional grating can optionally comprise a single unit. The surface of the single unit comprising the two-dimensional grating is coated with a material having a high refractive index, and the one or more detection probes are immobilized on the surface of the material having a high refractive index opposite of the single unit. The single unit can be comprised of a material selected from the group consisting of glass, plastic, and epoxy.

The biosensor can optionally comprise a cover layer on the surface of the two-dimensional grating opposite of the substrate layer. The one or more detection probes are immobilized on the surface of the cover layer opposite of the two-dimensional grating. The cover layer can comprise a material that has a lower refractive index than the high refractive index material of the two-dimensional grating. For example, a cover layer can comprise glass, epoxy, and plastic.

A two-dimensional grating can be comprised of a repeating pattern of shapes selected from the group consisting of lines, squares, circles, ellipses, triangles, trapezoids, sinusoidal waves, ovals, rectangles, and hexagons. The repeating pattern of shapes can be arranged in a linear grid, i.e., a grid of parallel lines, a rectangular grid, or a hexagonal grid. The two-dimensional grating can have a period of about 0.01 microns to about I micron and a depth of about 0.01 microns to about 1 micron.

To illustrate, biochemical interactions occurring on a surface of a calorimetric resonant optical biosensor embedded into a surface of a microarray slide, microtiter plate or other device, can be directly detected and measured on the sensor's surface without the use of fluorescent tags or calorimetric labels. The sensor surface contains an optical structure that, when illuminated with collimated white light, is designed to reflect only a narrow band of wavelengths (color). The narrow wavelength is described as a wavelength “peak.” The “peak wavelength value” (PWV) changes when biological material is deposited or removed from the sensor surface, such as when binding occurs. Such binding-induced change of PWV can be measured using a measurement instrument disclosed in US2003/0032039.

In one embodiment, the instrument illuminates the biosensor surface by directing a collimated white light on to the sensor structure. The illuminated light may take the form of a spot of collimated light. Alternatively, the light is generated in the form of a fan beam. The instrument collects light reflected from the illuminated biosensor surface. The instrument may gather this reflected light from multiple locations on the biosensor surface simultaneously. The instrument can include a plurality of illumination probes that direct the light to a discrete number of positions across the biosensor surface. The instrument measures the Peak Wavelength Values (PWVs) of separate locations within the biosensor-embedded microtiter plate using a spectrometer. In one embodiment, the spectrometer is a single-point spectrometer. Alternatively, an imaging spectrometer is used. The spectrometer can produce a PWV image map of the sensor surface. In one embodiment, the measuring instrument spatially resolves PWV images with less than 200 micron resolution.

In one embodiment, a subwavelength structured surface (SWS) may be used to create a sharp optical resonant reflection at a particular wavelength that can be used to track with high sensitivity the interaction of biological materials, such as specific binding substances or binding partners or both. A colormetric resonant diffractive grating surface acts as a surface binding platform for specific binding substances (such as immobilized capture agents of the instant invention). SWS is an unconventional type of diffractive optic that can mimic the effect of thin-film coatings. (Peng & Morris, “Resonant scattering from two-dimensional gratings,” J. Opt. Soc. Am. A, Vol. 13, No. 5, p. 993, May; Magnusson, & Wang, “New principle for optical filters,” Appl. Phys. Lett., 61, No. 9, p. 1022, August, 1992; Peng & Morris, “Experimental demonstration of resonant anomalies in diffraction from two-dimensional gratings,” Optics Letters, Vol. 21, No. 8, p. 549, April, 1996). A SWS structure contains a surface-relief, two-dimensional grating in which the grating period is small compared to the wavelength of incident light so that no diffractive orders other than the reflected and transmitted zeroth orders are allowed to propagate. A SWS surface narrowband filter can comprise a two-dimensional grating sandwiched between a substrate layer and a cover layer that fills the grating grooves. Optionally, a cover layer is not used. When the effective index of refraction of the grating region is greater than the substrate or the cover layer, a waveguide is created. When a filter is designed accordingly, incident light passes into the waveguide region. A two-dimensional grating structure selectively couples light at a narrow band of wavelengths into the waveguide. The light propagates only a short distance (on the order of 10-100 micrometers), undergoes scattering, and couples with the forward- and backward-propagating zeroth-order light. This sensitive coupling condition can produce a resonant grating effect on the reflected radiation spectrum, resulting in a narrow band of reflected or transmitted wavelengths (colors). The depth and period of the two-dimensional grating are less than the wavelength of the resonant grating effect.

The reflected or transmitted color of this structure can be modulated by the addition of molecules such as capture agents or their URS-containing binding partners or both, to the upper surface of the cover layer or the two-dimensional grating surface. The added molecules increase the optical path length of incident radiation through the structure, and thus modify the wavelength (color) at which maximum reflectance or transmittance will occur. Thus in one embodiment, a biosensor, when illuminated with white light, is designed to reflect only a single wavelength. When specific binding substances are attached to the surface of the biosensor, the reflected wavelength (color) is shifted due to the change of the optical path of light that is coupled into the grating. By linking specific binding substances to a biosensor surface, complementary binding partner molecules can be detected without the use of any kind of fluorescent probe or particle label. The detection technique is capable of resolving changes of, for example, about 0.1 nm thickness of protein binding, and can be performed with the biosensor surface either immersed in fluid or dried. This PWV change can be detected by a detection system consists of, for example, a light source that illuminates a small spot of a biosensor at normal incidence through, for example, a fiber optic probe. A spectrometer collects the reflected light through, for example, a second fiber optic probe also at normal incidence. Because no physical contact occurs between the excitation/detection system and the biosensor surface, no special coupling prisms are required. The biosensor can, therefore, be adapted to a commonly used assay platform including, for example, microtiter plates and microarray slides. A spectrometer reading can be performed in several milliseconds, thus it is possible to efficiently measure a large number of molecular interactions taking place in parallel upon a biosensor surface, and to monitor reaction kinetics in real time.

Various embodiments, variations of the biosensor described above can be found in US2003/0032039, incorporated herein by reference in its entirety.

One or more specific capture agents may be immobilized on the two-dimensional grating or cover layer, if present. Immobilization may occur by any of the above described methods. Suitable capture agents can be, for example, a nucleic acid, polypeptide, antigen, polyclonal antibody, monoclonal antibody, single chain antibody (scFv), F(ab) fragment, F(ab′)2 fragment, Fv fragment, small organic molecule, even cell, virus, or bacteria. A biological sample can be obtained and/or deribed from, for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, or prostatitc fluid. Preferably, one or more specific capture agents are arranged in a microarray of distinct locations on a biosensor. A microarray of capture agents comprises one or more specific capture agents on a surface of a biosensor such that a biosensor surface contains a plurality of distinct locations, each with a different capture agent or with a different amount of a specific capture agent. For example, an array can comprise 1, 10, 100, 1,000, 10,000, or 100,000 distinct locations. A biosensor surface with a large number of distinct locations is called a microarray because one or more specific capture agents are typically laid out in a regular grid pattern in x-y coordinates. However, a microarray can comprise one or more specific capture agents laid out in a regular or irregular pattern.

A microarray spot can range from about 50 to about 500 microns in diameter. Alternatively, a microarray spot can range from about 150 to about 200 microns in diameter. One or more specific capture agents can be bound to their specific URS-containing binding partners.

In one biosensor embodiment, a microarray on a biosensor is created by placing microdroplets of one or more specific capture agents onto, for example, an x-y grid of locations on a two-dimensional grating or cover layer surface. When the biosensor is exposed to a test sample comprising one or more URS binding partners, the binding partners will be preferentially attracted to distinct locations on the microarray that comprise capture agents that have high affinity for the URS binding partners. Some of the distinct locations will gather binding partners onto their surface, while other locations will not. Thus a specific capture agent specifically binds to its URS binding partner, but does not substantially bind other URS binding partners added to the surface of a biosensor. In an alternative embodiment, a nucleic acid microarray (such as an aptamer array) is provided, in which each distinct location within the array contains a different aptamer capture agent. By application of specific capture agents with a microarray spotter onto a biosensor, specific binding substance densities of 10,000 specific binding substances/in² can be obtained. By focusing an illumination beam of a fiber optic probe to interrogate a single microarray location, a biosensor can be used as a label-free microarray readout system.

For the detection of URS binding partners at concentrations of less than about 0.1 ng/ml, one may amplify and transduce binding partners bound to a biosensor into an additional layer on the biosensor surface. The increased mass deposited on the biosensor can be detected as a consequence of increased optical path length. By incorporating greater mass onto a biosensor surface, an optical density of binding partners on the surface is also increased, thus rendering a greater resonant wavelength shift than would occur without the added mass. The addition of mass can be accomplished, for example, enzymatically, through a “sandwich” assay, or by direct application of mass (such as a second capture agent specific for the URS peptide) to the biosensor surface in the form of appropriately conjugated beads or polymers of various size and composition. Since the capture agents are URS-specific, multiple capture agents of different types and specificity can be added together to the captured URSs. This principle has been exploited for other types of optical biosensors to demonstrate sensitivity increases over 1500× beyond sensitivity limits achieved without mass amplification. See, e.g., Jenison et al., “Interference-based detection of nucleic acid targets on optically coated silicon,” Nature Biotechnology, 19: 62-65, 2001.

In an alternative embodiment, a biosensor comprises volume surface-relief volume diffractive structures (a SRVD biosensor). SRVD biosensors have a surface that reflects predominantly at a particular narrow band of optical wavelengths when illuminated with a broad band of optical wavelengths. Where specific capture agents and/or URS binding partners are immobilized on a SRVD biosensor, the reflected wavelength of light is shifted. One-dimensional surfaces, such as thin film interference filters and Bragg reflectors, can select a narrow range of reflected or transmitted wavelengths from a broadband excitation source. However, the deposition of additional material, such as specific capture agents and/or URS binding partners onto their upper surface results only in a change in the resonance linewidth, rather than the resonance wavelength. In contrast, SRVD biosensors have the ability to alter the reflected wavelength with the addition of material, such as specific capture agents and/or binding partners to the surface.

A SRVD biosensor comprises a sheet material having a first and second surface. The first surface of the sheet material defines relief volume diffraction structures. Sheet material can comprise, for example, plastic, glass, semiconductor wafer, or metal film. A relief volume diffractive structure can be, for example, a two-dimensional grating, as described above, or a three-dimensional surface-relief volume diffractive grating. The depth and period of relief volume diffraction structures are less than the resonance wavelength of light reflected from a biosensor. A three-dimensional surface-relief volume diffractive grating can be, for example, a three-dimensional phase-quantized terraced surface relief pattern whose groove pattern resembles a stepped pyramid. When such a grating is illuminated by a beam of broadband radiation, light will be coherently reflected from the equally spaced terraces at a wavelength given by twice the step spacing times the index of refraction of the surrounding medium. Light of a given wavelength is resonantly diffracted or reflected from the steps that are a half-wavelength apart, and with a bandwidth that is inversely proportional to the number of steps. The reflected or diffracted color can be controlled by the deposition of a dielectric layer so that a new wavelength is selected, depending on the index of refraction of the coating.

A stepped-phase structure can be produced first in photoresist by coherently exposing a thin photoresist film to three laser beams, as described previously. See e.g., Cowen, “The recording and large scale replication of crossed holographic grating arrays using multiple beam interferometry,” in International Conference on the Application, Theory, and Fabrication of Periodic Structures, Diffraction Gratings, and Moire Phenomena II, Lerner, ed., Proc. Soc. Photo-Opt. Instrum. Eng., 503, 120-129, 1984; Cowen, “Holographic honeycomb microlens,” Opt. Eng. 24, 796-802 (1985); Cowen & Slafer, “The recording and replication of holographic micropatterns for the ordering of photographic emulsion grains in film systems,” J Imaging Sci. 31, 100-107, 1987. The nonlinear etching characteristics of photoresist are used to develop the exposed film to create a three-dimensional relief pattern. The photoresist structure is then replicated using standard embossing procedures. For example, a thin silver film may be deposited over the photoresist structure to form a conducting layer upon which a thick film of nickel can be electroplated. The nickel “master” plate is then used to emboss directly into a plastic film, such as vinyl, that has been softened by heating or solvent. A theory describing the design and fabrication of three-dimensional phase-quantized terraced surface relief pattern that resemble stepped pyramids is described: Cowen, “Aztec surface-relief volume diffractive structure,” J. Opt. Soc. Am. A, 7:1529 (1990). An example of a three-dimensional phase-quantized terraced surface relief pattern may be a pattern that resembles a stepped pyramid. Each inverted pyramid is approximately 1 micron in diameter. Preferably, each inverted pyramid can be about 0.5 to about 5 microns diameter, including for example, about 1 micron. The pyramid structures can be close-packed so that a typical microarray spot with a diameter of 150-200 microns can incorporate several hundred stepped pyramid structures. The relief volume diffraction structures have a period of about 0.1 to about 1 micron and a depth of about 0.1 to about 1 micron.

One or more specific binding substances, as described above, are immobilized on the reflective material of a SRVD biosensor. One or more specific binding substances can be arranged in microarray of distinct locations, as described above, on the reflective material.

A SRVD biosensor reflects light predominantly at a first single optical wavelength when illuminated with a broad band of optical wavelengths, and reflects light at a second single optical wavelength when one or more specific binding substances are immobilized on the reflective surface. The reflection at the second optical wavelength results from optical interference. A SRVD biosensor also reflects light at a third single optical wavelength when the one or more specific capture agents are bound to their respective URS binding partners, due to optical interference. Readout of the reflected color can be performed serially by focusing a microscope objective onto individual microarray spots and reading the reflected spectrum with the aid of a spectrograph or imaging spectrometer, or in parallel by, for example, projecting the reflected image of the microarray onto an imaging spectrometer incorporating a high resolution color CCD camera.

A SRVD biosensor can be manufactured by, for example, producing a metal master plate, and stamping a relief volume diffractive structure into, for example, a plastic material like vinyl. After stamping, the surface is made reflective by blanket deposition of, for example, a thin metal film such as gold, silver, or aluminum. Compared to MEMS-based biosensors that rely upon photolithography, etching, and wafer bonding procedures, the manufacture of a SRVD biosensor is very inexpensive.

A SWS or SRVD biosensor embodiment can comprise an inner surface. In one preferred embodiment, such an inner surface is a bottom surface of a liquid-containing vessel. A liquid-containing vessel can be, for example, a microtiter plate well, a test tube, a petri dish, or a microfluidic channel. In one embodiment, a SWS or SRVD biosensor is incorporated into a microtiter plate. For example, a SWS biosensor or SRVD biosensor can be incorporated into the bottom surface of a microtiter plate by assembling the walls of the reaction vessels over the resonant reflection surface, so that each reaction “spot” can be exposed to a distinct test sample. Therefore, each individual microtiter plate well can act as a separate reaction vessel. Separate chemical reactions can, therefore, occur within adjacent wells without intermixing reaction fluids and chemically distinct test solutions can be applied to individual wells.

This technology is useful in applications where large numbers of biomolecular interactions are measured in parallel, particularly when molecular labels would alter or inhibit the functionality of the molecules under study. High-throughput screening of pharmaceutical compound libraries with protein targets, and microarray screening of protein-protein interactions for proteomics are examples of applications that require the sensitivity and throughput afforded by the compositions and methods of the invention.

Unlike surface plasmon resonance, resonant mirrors, and waveguide biosensors, the described compositions and methods enable many thousands of individual binding reactions to take place simultaneously upon the biosensor surface. This technology is useful in applications where large numbers of biomolecular interactions are measured in parallel (such as in an array), particularly when molecular labels alter or inhibit the functionality of the molecules under study. These biosensors are especially suited for high-throughput screening of pharmaceutical compound libraries with protein targets, and microarray screening of protein-protein interactions for proteomics. A biosensor of the invention can be manufactured, for example, in large areas using a plastic embossing process, and thus can be inexpensively incorporated into common disposable laboratory assay platforms such as microtiter plates and microarray slides.

Other similar biosensors may also be used in the instant invention. Numerous biosensors have been developed to detect a variety of biomolecular complexes including oligonucleotides, antibody-antigen interactions, hormone-receptor interactions, and enzyme-substrate interactions. In general, these biosensors consist of two components: a highly specific recognition element and a transducer that converts the molecular recognition event into a quantifiable signal. Signal transduction has been accomplished by many methods, including fluorescence, interferometry (Jenison et al., “Interference-based detection of nucleic acid targets on optically coated silicon,” Nature Biotechnology, 19, p. 62-65; Lin et al., “A porous silicon-based optical interferometric biosensor,” Science, 278, p. 840-843, 1997), and gravimetry (A. Cunningham, Bioanalytical Sensors, John Wiley & Sons (1998)). Of the optically-based transduction methods, direct methods that do not require labeling of analytes with fluorescent compounds are of interest due to the relative assay simplicity and ability to study the interaction of small molecules and proteins that are not readily labeled.

These direct optical methods include surface plasmon resonance (SPR) (Jordan & Corn, “Surface Plasmon Resonance Imaging Measurements of Electrostatic Biopolymer Adsorption onto Chemically Modified Gold Surfaces,” Anal. Chem., 69:1449-1456 (1997); plasmom-resonant particles (PRPs) (Schultz et al., Proc. Nat. Acad. Sci., 97: 996-1001 (2000); grating couplers (Morhard et al., “Innnobilization of antibodies in micropattems for cell detection by optical diffraction,” Sensors and Actuators B, 70, p. 232-242, 2000); ellipsometry (Jin et al., “A biosensor concept based on imaging ellipsometry for visualization of biomolecular interactions,” Analytical Biochemistry, 232, p. 69-72, 1995), evanascent wave devices (Huber et al., “Direct optical immunosensing (sensitivity and selectivity),” Sensors and Actuators B, 6, p.122.126, 1992), resonance light scattering (Bao et al., Anal. Chem., 74:1792-1797 (2002), and reflectometry (Brecht & Gauglitz, “Optical probes and transducers,” Biosensors and Bioelectronics, 10, p. 923-936, 1995). Changes in the optical phenomenon of surface plasmon resonance (SPR) can be used as an indication of real-time reactions between biological molecules. Theoretically predicted detection limits of these detection methods have been determined and experimentally confirmed to be feasible down to diagnostically relevant concentration ranges.

Surface plasmon resonance (SPR) has been successfully incorporated into an immunosensor format for the simple, rapid, and nonlabeled assay of various biochemical analytes. Proteins, complex conjugates, toxins, allergens, drugs, and pesticides can be determined directly using either natural antibodies or synthetic receptors with high sensitivity and selectivity as the sensing element. Immunosensors are capable of real-time monitoring of the antigen-antibody reaction. A wide range of molecules can be detected with lower limits ranging between 10⁻⁹ and 10⁻¹³ mol/L. Several successful commercial developments of SPR immunosensors are available and their web pages are rich in technical information. Wayne et al. (Methods 22: 77-91, 2000) reviewed and highlighted many recent developments in SPR-based immunoassay, functionalizations of the gold surface, novel receptors in molecular recognition, and advanced techniques for sensitivity enhancement.

Utilization of the optical phenomenon surface plasmon resonance (SPR) has seen extensive growth since its initial observation by Wood in 1902 (Phil. Mag. 4 (1902), pp. 396-402). SPR is a simple and direct sensing technique that can be used to probe refractive index (η) changes that occur in the very close vicinity of a thin metal film surface (Otto Z. Phys. 216 (1968), p. 398). The sensing mechanism exploits the properties of an evanescent field generated at the site of total internal reflection. This field penetrates into the metal film, with exponentially decreasing amplitude from the glass-metal interface. Surface plasmons, which oscillate and propagate along the upper surface of the metal film, absorb some of the plane-polarized light energy from this evanescent field to change the total internal reflection light intensity Iᵣ. A plot of Iᵣ versus incidence (or reflection) angle θ produces an angular intensity profile that exhibits a sharp dip. The exact location of the dip minimum (or the SPR angle θᵣ) can be determined by using a polynomial algorithm to fit the Iᵣ signals from a few diodes close to the minimum. The binding of molecules on the upper metal surface causes a change in η of the surface medium that can be observed as a shift in θᵣ.

The potential of SPR for biosensor purposeswas realized in 1982-1983 by Liedberg et al., who adsorbed an immunoglobulin G (IgG) antibody overlayer on the gold sensing film, resulting in the subsequent selective binding and detection of IgG (Nylander et al., Sens. Actuators 3 (1982), pp. 79-84; Liedberg et al., Sens. Actuators 4 (1983), pp. 229-304). The principles of SPR as a biosensing technique have been reviewed previously (Daniels et al., Sens. Actuators 15 (1988), pp. 11-18; VanderNoot and Lai, Spectroscopy 6 (1991), pp. 28-33; Lundström Biosens. Bioelectron. 9 (1994), pp. 725-736; Liedberg et al., Biosens. Bioelectron. 10 (1995); Morgan et al., Clin. Chem. 42 (1996), pp. 193-209; Tapuchi et al., S. Afr. J. Chem. 49 (1996), pp. 8-25). Applications of SPR to biosensing were demonstrated for a wide range of molecules, from virus particles to sex hormonebinding globulin and syphilis. Most importantly, SPR has an inherent advantage over other types of biosensors in its versatility and capability of monitoring binding interactions without the need for fluorescence or radioisotope labeling of the biomolecules. This approach has also shown promise in the real-time determination of concentration, kinetic constant, and binding specificity of individual biomolecular interaction steps. Antibody-antigen interactions, peptide/protein-protein interactions, DNA hybridization conditions, biocompatibility studies of polymers, biomolecule-cell receptor interactions, and DNA/receptor-ligand interactions can all be analyzed (Pathak and Savelkoul, Immunol. Today 18 (1997), pp. 464-467). Commercially, the use of SPR-based immunoassay has been promoted by companies such as Biacore (Uppsala, Sweden) (Jönsson et al., Ann. Biol. Clin. 51 (1993), pp. 19-26), Windsor Scientific (U.K.) (WWW URL for Windsor Scientific IBIS Biosensor), Quantech (Minnesota) (WWW URL for Quantech), and Texas Instruments (Dallas, Tex.) (WWW URL for Texas Instruments).

In yet another embodiment, a fluorescent polymer superquenching-based bioassays as disclosed in WO 02/074997 may be used for detecting binding of the unlabeled URS to its capture agents. In this embodiment, a capture agent that is specific for both a target URS peptide and a chemical moiety is used. The chemical moiety includes (a) a recognition element for the capture agent, (b) a fluorescent property-altering element, and (c) a tethering element linking the recognition element and the property-altering element. A composition comprising a fluorescent polymer and the capture agent are co-located on a support. When the chemical moiety is bound to the capture agent, the property-altering element of the chemical moiety is sufficiently close to the fluorescent polymer to alter (quench) the fluorescence emitted by the polymer. When an analyte sample is introduced, the target URS peptide, if present, binds to the capture agent, thereby displacing the chemical moiety from the receptor, resulting in de-quenching and an increase of detected fluorescence. Assays for detecting the presence of a target biological agent are also disclosed in the application.

In another related embodiment, the binding event between the capture agents and the URS can be detected by using a water-soluble luminescent quantum dot as described in US2003/0008414A1. In one embodiment, a water-soluble luminescent semiconductor quantum dot comprises a core, a cap and a hydrophilic attachment group. The “core” is a nanoparticle-sized semiconductor. While any core of the IIB-VIB, IIIB-VB or IVB-IVB semiconductors can be used in this context, the core must be such that, upon combination with a cap, a luminescent quantum dot results. A IIB-VIB semiconductor is a compound that contains at least one element from Group IEB and at least one element from Group VIB of the periodic table, and so on. Preferably, the core is a IIB-VIB, IIIB-VB or IVB-IVB semiconductor that ranges in size from about 1 nm to about 10 nm. The core is more preferably a IIB-VIB semiconductor and ranges in size from about 2 nm to about 5 nm. Most preferably, the core is CdS or CdSe. In this regard, CdSe is especially preferred as the core, in particular at a size of about 4.2 nm.

The “cap” is a semiconductor that differs from the semiconductor of the core and binds to the core, thereby forming a surface layer on the core. The cap must be such that, upon combination with a given semiconductor core, results in a luminescent quantum dot. The cap should passivate the core by having a higher band gap than the core. In this regard, the cap is preferably a IIB-VIB semiconductor of high band gap. More preferably, the cap is ZnS or CdS. Most preferably, the cap is ZnS. In particular, the cap is preferably ZnS when the core is CdSe or CdS and the cap is preferably CdS when the core is CdSe.

The “attachment group” as that term is used herein refers to any organic group that can be attached, such as by any stable physical or chemical association, to the surface of the cap of the luminescent semiconductor quantum dot and can render the quantum dot water-soluble without rendering the quantum dot no longer luminescent. Accordingly, the attachment group comprises a hydrophilic moiety. Preferably, the attachment group enables the hydrophilic quantum dot to remain in solution for at least about one hour, one day, one week, or one month. Desirably, the attachment group is attached to the cap by covalent bonding and is attached to the cap in such a manner that the hydrophilic moiety is exposed. Preferably, the hydrophilic attachment group is attached to the quantum dot via a sulfur atom. More preferably, the hydrophilic attachment group is an organic group comprising a sulfur atom and at least one hydrophilic attachment group. Suitable hydrophilic attachment groups include, for example, a carboxylic acid or salt thereof, a sulfonic acid or salt thereof, a sulfamic acid or salt thereof, an amino substituent, a quaternary ammonium salt, and a hydroxy. The organic group of the hydrophilic attachment group of the present invention is preferably a C1-C6 alkyl group or an aryl group, more preferably a C1-C6 alkyl group, even more prefeably a C1-C3 alkyl group. Therefore, in a preferred embodiment, the attachment group of the present invention is a thiol carboxylic acid or thiol alcohol. More preferably, the attachment group is a thiol carboxylic acid. Most preferably, the attachment group is mercaptoacetic acid.

Accordingly, a preferred embodiment of a water-soluble luminescent semiconductor quantum dot is one that comprises a CdSe core of about 4.2 nm in size, a ZnS cap and an attachment group. Another preferred embodiment of a watersoluble luminescent semiconductor quantum dot is one that comprises a CdSe core, a ZnS cap and the attachment group mercaptoacetic acid. An especially preferred water-soluble luminescent semiconductor quantum dot comprises a CdSe core of about 4.2 nm, a ZnS cap of about 1 nm and a mercaptoacetic acid attachment group.

The capture agent of the instant invention can be attached to the quantum dot via the hydrophilic attachment group and forms a conjugate. The capture agent can be attached, such as by any stable physical or chemical association, to the hydrophilic attachment group of the water-soluble luminescent quantum dot directly or indirectly by any suitable means, through one or more covalent bonds, via an optional linker that does not impair the function of the capture agent or the quantum dot. For example, if the attachment group is mercaptoacetic acid and a nucleic acid biomolecule is being attached to the attachment group, the linker preferably is a primary amine, a thiol, streptavidin, neutravidin, biotin, or a like molecule. If the attachment group is mercaptoacetic acid and a protein biomolecule or a fragment thereof is being attached to the attachment group, the linker preferably is strepavidin, neutravidin, biotin, or a like molecule.

By using the quantum dot-capture agent conjugate, a URS-containing sample, when contacted with a conjugate as described above, will promote the emission of luminescence when the capture agent of the conjugate specifically binds to the URS peptide. This is particularly useful when the capture agent is a nucleic acid aptamer or an antibody. When the aptamer is used, an alternative embodiment may be employed, in which a fluorescent quencher may be positioned adjacent to the quantum dot via a self-pairing stem-loop structure when the aptamer is not bound to a URS-containing sequence. When the aptamer binds to the URS, the stem-loop structure is opened, thus releasing the quenching effect and generates luminiscence.

In another related embodiment, arrays of nanosensors comprising nanowires or nanotubes as described in US2002/0117659A1 may be used for detection and/or quantitation of URS-capture agent interaction. Briefly, a “nanowire” is an elongated nanoscale semiconductor, which can have a cross-sectional dimension of as thin as 1 nanometer. Similarly, a “nanotube” is a nanowire that has a hollowed-out core, and includes those nanotubes know to those of ordinary skill in the art. A “wire” refers to any material having a conductivity at least that of a semiconductor or metal. These nanowires/nanotubes may be used in a system constructed and arranged to determine an analyte (e.g., URS peptide) in a sample to which the nanowire(s) is exposed. The surface of the nanowire is functionalized by coating with a capture agent. Binding of an analyte to the functionalized nanowire causes a detectable change in electrical conductivity of the nanowire or optical properties. Thus, presence of the analyte can be determined by determining a change in a characteristic in the nanowire, typically an electrical characteristic or an optical characteristic. A variety of biomolecular entities can be used for coating, including, but not limited to, amino acids, proteins, sugars, DNA, antibodies, antigens, and enzymes, etc. For more details such as construction of nanowires, functionalization with various biomolecules (such as the capture agents of the instant invention), and detection in nanowire devices, see US2002/0117659A1 (incorporated by reference). Since multiple nanowires can be used in parelle, each with a different capture agent as the functionalized group, this technology is ideally suited for large scale arrayed detection of URS-containing peptides in biological samples without the need to label the URS peptides. This nanowire detection technology has been successfully used to detect pH change (H⁺ binding), biotin-streptavidin binding, antibody-antigen binding, metal (Ca²⁺) binding with picomolar sensitivity and in real time (Cui et al., Science 293: 1289-1292).

Matrix-assisted laser desorption/ionization time-of-flight mass spectrometry (MALDI-TOF MS), uses a laser pulse to desorb proteins from the surface followed by mass spectrometry to identify the molecular weights of the proteins (Gilligan et al., Mass spectrometry after capture and small-volume elution of analyte from a surface plasmon resonance biosensor. Anal. Chem. 74 (2002), pp. 2041-2047). Because this method only measures the mass of proteins at the interface, and because the desorption protocol is sufficiently mild that it does not result in fragmentation, MALDI can provide straightforward useful information such as confirming the identity of the bound URS peptide, or any enzymatic modification of a URS peptide. For this matter, MALDI can be used to identify proteins that are bound to immobilized capture agents. An important technique for identifying bound proteins relies on treating the array (and the proteins that are selectively bound to the array) with proteases and then analyzing the resulting peptides to obtain sequence data.

IV. Samples and Their Preparation

The capture agents or an array of capture agents typically are contacted with a sample, e.g., a biological fluid, a water sample, or a food sample, which has been fragmented to generate a collection of peptides, under conditions suitable for binding a URS corresponding to a protein of interest.

Samples to be assayed using the capture agents of the present invention may be drawn from various physiological, environmental or artificial sources. In particular, physiological samples such as body fluids or tissue samples of a patient or an organism may be used as assay samples. Such fluids include, but are not limited to, saliva, mucous, sweat, whole blood, serum, urine, amniotic fluid, genital fluids, fecal material, marrow, plasma, spinal fluid, pericardial fluids, gastric fluids, abdominal fluids, peritoneal fluids, pleural fluids and extraction from other body parts, and secretion from other glands. Alternatively, biological samples drawn from cells taken from the patient or grown in culture may be employed. Such samples include supernatants, whole cell lysates, or cell fractions obtained by lysis and fractionation of cellular material. Extracts of cells and fractions thereof, including those directly from a biological entity and those grown in an artificial environment, can also be used. In addition, a biological sample can be obtained and/or deribed from, for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, or prostatitc fluid.

The sample may be pre treated to remove extraneous materials, stabilized, buffered, preserved, filtered, or otherwise conditioned as desired or necessary. Proteins in the sample typically are fragmented, either as part of the methods of the invention or in advance of performing these methods. Fragmentation can be performed using any art-recognized desired method, such as by using chemical cleavage (e.g., cyanogen bromide); enzymatic means (e.g., using a protease such as trypsin, chymotrypsin, pepsin, papain, carboxypeptidase, calpain, subtilisin, gluc-C, endo lys-C and proteinase K, or a collection or sub-collection thereof); or physical means (e.g., fragmentation by physical shearing or fragmentation by sonication). As used herein, the terms “fragmentation” “cleavage,” “proteolytic cleavage,” “proteolysis” “restriction” and the like are used interchangeably and refer to scission of a chemical bond, typically a peptide bond, within proteins to produce a collection of peptides (i.e., protein fragments).

The purpose of the fragmentation is to generate peptides comprising URS which are soluble and available for binding with a capture agent. In essence, the sample preparation is designed to assure to the extent possible that all URS present on or within relevant proteins that may be present in the sample are available for reaction with the capture agents. This strategy can avoid many of the problems encountered with previous attempts to design protein chips caused by protein-protein complexation, post translational modifications and the like.

In one embodiment, the sample of interest is treated using a pre-determined protocol which: (A) inhibits masking of the target protein caused by target protein-protein non covalent or covalent complexation or aggregation, target protein degradation or denaturing, target protein post-translational modification, or environmentally induced alteration in target protein tertiary structure, and (B) fragments the target protein to, thereby, produce at least one peptide epitope (i.e., a URS) whose concentration is directly proportional to the true concentration of the target protein in the sample. The sample treatment protocol is designed and empirically tested to result reproducibly in the generation of a URS that is available for reaction with a given capture agent. The treatment can involve protein separations; protein fractionations; solvent modifications such as polarity changes, osmolarity changes, dilutions, or pH changes; heating; freezing; precipitating; extractions; reactions with a reagent such as an endo-, exo- or site specific protease; non proteolytic digestion; oxidations; reductions; neutralization of some biological activity, and other steps known to one of skill in the art.

For example, the sample may be treated with an alkylating agent and a reducing agent in order to prevent the formation of dimers or other aggregates through disulfide/dithiol exchange. The sample of URS-containing peptides may also be treated to remove secondary modifications, including but are not limited to, phosphorylation, methylation, glycosylation, acetylation, prenylation, using, for example, respective modification-specific enzymes such as phosphatases, etc.

In one embodiment, proteins of a sample will be denatured, reduced and/or alkylated, but will not be proteolytically cleaved. Proteins can be denatured by thermal denaturation or organic solvents, then subjected to direct detection or optionally, further proteolytic cleavage.

Fractionation may be performed using any single or multidimentional chromatography, such as reversed phase chromatography (RPC), ion exchange chromatography, hydrophobic interaction chromatography, size exclusion chromatography, or affinity fractionation such as immunoaffinity and immobilized metal affinity chromatography. Preferably, the fractionation involves surface-mediated selection strategies. Electrophoresis, either slab gel or capillary electrophoresis, can also be used to fractionate the peptides in the sample. Examples of slab gel electrophoretic methods include sodium dodecyl sulfate polyacrylamide gel electrophoresis (SDS-PAGE) and native gel electrophoresis. Capillary electrophoresis methods that can be used for fractionation include capillary gel electrophoresis (CGE), capillary zone electrophoresis (CZE) and capillary electrochromatography (CEC), capillary isoelectric focusing, immobilized metal affinity chromatography and affinity electrophoresis.

Protein precipitation may be performed using techniques well known in the art. For example, precipitation may be achieved using known precipitants, such as potassium thiocyanate, trichloroacetic acid and ammonium sulphate.

Subsequent to fragmentation, the sample may be contacted with the capture agents of the present invention, e.g., capture agents immobilized on a planar support or on a bead, as described herein. Alternatively, the fragmented sample (containing a collection of peptides) may be fractionated based on, for example, size, post-translational modifications (e.g., glycosylation or phosphorylation) or antigenic properties, and then contacted with the capture agents of the present invention, e.g., capture agents immobilized on a planar support or on a bead.

V. Selection of URS

The URS of the instant invention can be selected in various ways. In the simplest embodiment, the URS for a given organism or biological sample can be generated or identified by a brute force search of the relevant database, using all theoretically possible URS with a given length. For example, to identify URS of 5 amino acids in length (a total of 3.2 million possible URS candidates, see table 2.2.2 below), each of the 3.2 million candidates may be used as a query sequence to search against the human proteom as described below. Any candidate that has more than one hit (found in two or more proteins) is immediately eliminated before further searching is done. At the end of the search, a list of human proteins that have one or more URSs can be obtained (see Example 1 below). The same or similar procedure can be used for any pre-determined organism or database.

For example, URSs for each human protein can be identified using the following procedure. A Perl program is developed to calculate the occurrence of all possible peptides, given by 20^{N}, of defined length N (amino acids) in human proteins. For example, the total tag space is 160,000 (20⁴) for tetramer peptides, 3.2 M (20⁵) for pentamer peptides, and 64 M (20⁶) for hexamer peptides, so on. Predicted human protein sequences are analyzed for the presence or absence of all possible peptides of N amino acids. URS are the peptide sequences that occur only once in the human proteome. Thus the presence of a specific URS is an intrinsic property of the protein sequence and is operational independent. According to this approach, a definitive set of URSs can be defined and used regardless of the sample processing procedure (operational independence).

In one embodiment, to speed up the searching process, computer algorithms may be developed or modified to eliminate unnecessary searches before the actual search begins.

Using the example above, two highly related (say differ only in a few amino acid positions) human proteins may be aligned, and a large number of candidate URS can be eliminated based on the sequence of the identical regions. For example, if there is a stretch of identical sequence of 20 amino acids, then sixteen 5-amino acid URSs can be eliminated without searching, by virtue of their simultaneous appearance in two non-identical human proteins. This elimination process can be continued using as many highly related protein pairs or families as possible, such as the evolutionary conserved proteins such as histones, globins, etc.

In another embodiment, the identified URS for a given protein may be rankordered based on certain criteria, so that higher ranking URSs are preferred to be used in generating specific capture agents.

For example, certain URS may naturally exist on protein surface, thus making good candidates for being a soluble peptide when digested by a protease. On the other hand, certain URS may exist in an internal or core region of a protein, and may not be readily soluble even after digestion. Such solubility property may be evaluated by avilable softwares. The solvent accessibility method described in Boger, J., Emini, E. A. & Schmidt, A., Surface probability profile-An heuristic approach to the selection of synthetic peptide antigens, Reports on the Sixth International Congress in Immunology (Toronto) 1986 p.250 also may be used to identify URSs that are located on the surface of the protein of interest. The package MOLMOL (Koradi, R. et al. (1996) J. Mol. Graph. 14:51-55) and Eisenhaber's ASC method (Eisenhaber and Argos (1993) J. Comput. Chem. 14:1272-1280; Eisenhaber et al. (1995) J. Comput. Chem. 16:273-284) may also be used. Surface URSs generally have higher ranking than internal URSs. In one embodiment, the log P or log D values that can be calculated for a URS, or proteolytic fragment containing a URS, can be calculated and used to rank order the URS's based on likely solubility under conditions that a protein sample is to be contacted with a capture agent.

Any URS may also be associated with an annotation, which may contain useful information such as: whether the URS may be desctroyed by a certain protease (such as trypsin), whether it is likely to appear on a digested peptide with a relatively rigid or flexible structure, etc. These characteristics may help to rank order the URSs for use if generating specific capture agents, especially when there are a large number of URSs associated with a given protein. Since URS may change depending on particular use in a given organism, ranking order may change depending on specific usages. A URS may be low ranking due to its probability of being destroyed by a certain protease may rank higher in a different fragmentation scheme using a different protease.

In another embodiment, the computational algorithm for selecting optimal URS from a protein for antibody generation takes antibody-peptide interaction data into consideration. A process such as Nearest-Neighbor Analysis (NNA), can be used to select most unique URS for each protein. Each URS in a protein is given a relative score, or URS Uniqueness Index, that is based on the number of nearest neighbors it has. The higher the URS Uniqueness Index, the more unique the URS is. The URS Uniqueness Index can be calculated using an Amino Acid Replacement Matrix such as the one in Table VIII of Getzoff, E D, Tainer J A and Lemer R A. The chemistry and meachnism of antibody binding to protein antigens. 1988. Advances. Immunol. 43: 1-97. In this matrix, the replaceability of each amino acid by the remaining 19 amino acids was calculated based on experimental data on antibody cross-reactivity to a large number of peptides of single mutations (replacing each amino acid in a peptide sequence by the remaining 19 amino acids). For example, each octamer URS from a protein is compared to 8.7 million octamers present in human proteome and a URS Uniqueness Index is calculated. This process not only selects the most unique URS for particular protein, it also identifies Nearest Neighbor Peptides for this URS. This becomes important for defining cross-reactivity of URS-specific antibodies since Nearest Neighbor Peptides are the ones most likely will cross-react with particular antibody.

Besides URS Uniqueness Index, the following parameters for each URS may also be calculated and help to rank the URSs:

a) URS Solubility Index: which involves calculating LogP and LogD of the URS.

b) URS Hydrophobicity & water accessibility: only hydrophilic peptides and peptides with good water accessibility will be selected.

c) URS Length: since longer peptides tend to have conformations in solution, we use URS peptides with defined length of 8 amino acids. URS-specific antibodies will have better defined specificity due to limited number of epitopes in a shorter peptide sequences. This is very important for multiplexing assays using these antibodies. In one embodiment, only antibodies generated by this way will be used for multiplexing assays.

d) Evolutionary Conservation Index: each human URS will be compared with other species to see whether a URS sequence is conserved cross species. Ideally, URS with minimal conservation, for example, between mouse and human sequences will be selected. This will maximize the possibility to generate good immunoresponse and monoclonal antibodies in mouse.

A. Post-Translational Modifications

The subject computer generated URS's can also be analyzed according to the likely presence or absence of post-translational modifications. More than 100 different such modifications of amino acid residues are known, examples include but are not limited to acetylation, amidation, deamidation, prenylation (such as farnesylation or geranylation), formylation, glycosylation, hydroxylation, methylation, myristoylation, phosphorylation, ubiquitination, ribosylation and sulphation. Sequence analysis softwares which are capable of determining putative post-translational modification in a given amino acid sequence include the NetPhos server which produces neural network predictions for serine, threonine and tyrosine phosphorylation sites in eukaryotic proteins (available through http://www.cbs.dtu.dk/services/Net- Phos/), GPI Modification Site Prediction (available through http://mendel.imp.univie.ac.at/gpi) and the ExPASy proteomics server for total protein analysis (available through www.expasy.ch/tools/)

In certain embodiments, preferred URS moieties are those lacking any posttranslational modification sites, since post-translationally modified amino acid sequences may complicate sample preparation and/or interaction with a capture agent. Notwithstanding the above, capture agents that can discriminate between post-translationally forms of a URS, which may indicate a biological activity of the polypeptide-of-interest, can be generated and used in the present invention. A very common example is the phosphorylation of OH group of the amino acid side chain of a serine, a threonine, or a tyrosine group in a polypeptide. Depending on the polypeptide, this modification can increase or decrease its functional activity. In one embodiment, the subject invention provides an array of capture agents that are variegated so as to provide discriminatory binding and identification of various post-translationally modified forms of one or more proteins.

VI. Applications of the Invention

A. Investigative and Diagnostic Applications

The capture agents of the present invention provide a powerful tool in probing living systems and in diagnostic applications (e.g., clinical, environmental and industrial, and food safety diagnostic applications). For clinical diagnostic applications, the capture agents are designed such that they bind to one or more URS corresponding to one or more diagnostic targets (e.g., a disease related protein, collection of proteins, or pattern of proteins). Specific individual disease related proteins include, for example, prostate-specific antigen (PSA), prostatic acid phosphatase (PAP) or prostate specific membrane antigen (PSMA) (for diagnosing prostate cancer); Cyclin E for diagnosing breast cancer; Annexin, e.g., Annexin V (for diagnosing cell death in, for example, cancer, ischemia, or transplant rejection); or β-amyloid plaques (for diagnosing Alzheimer's Disease).

Thus, unique recognition sequences and the capture agents of the present invention may be used as a source of surrogate markers. For example, they can be used as markers of disorders or disease states, as markers for precursors of disease states, as markers for predisposition of disease states, as markers of drug activity, or as markers of the pharmacogenomic profile of protein expression.

As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the causation of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using a URS corresponding to a protein associated with a cardiovascular disease as a surrogate marker, and an analysis of HIV infection may be made using a URS corresponding to an HIV protein as a surrogate marker, well in advance of the undesirable clinical outcomes of myocardial infarction or fully-developed AIDS). Examples of the use of surrogate markers in the art include: Koomen et al. (2000) J. Mass. Spectrom. 35:258-264; and James (1994) AIDS Treatment News Archive 209.

Perhaps the most significant use of the invention is that it enables practice of a powerful new protein expression analysis technique: analyses of samples for the presence of specific combinations of proteins and specific levels of expression of combinations of proteins. This is valuable in molecular biology investigations generally, and particularly in development of novel assays. Thus, this invention permits one to identify proteins, groups of proteins, and protein expression patterns present in a sample which are characteristic of some disease, physiologic state, or species identity. Such multiparametric assay protocols may be particularly informative if the proteins being detected are from disconnected or remotely connected pathways. For example, the invention might be used to compare protein expression patterns in tissue, urine, or blood from normal patients and cancer patients, and to discover that in the presence of a particular type of cancer a first group of proteins are expressed at a higher level than normal and another group are expressed at a lower level. As another example, the protein chips might be used to survey protein expression levels in various strains of bacteria, to discover patterns of expression which characterize different strains, and to determine which strains are susceptible to which antibiotic. Furthermore, the invention enables production of specialty assay devices comprising arrays or other arrangements of capture agents for detecting specific patterns of specific proteins. Thus, to continue the example, in accordance with the practice of the invention, one can produce a chip which can be exposed to a cell lysate preparation from a patient or a body fluid to reveal the presence or absence or pattern of expression informative that the patient is cancer free, or is suffering from a particular cancer type. Alternatively, one might produce a protein chip that would be exposed to a sample and read to indicate the species of bacteria in an infection and the antibiotic that will destroy it.

A junction URS is a peptide which spans the region of a protein corresponding to a splice site of the RNA which encodes it. Capture agents designed to bind to a junction URS may be included in such analyses to detect splice variants as well as gene fusions generated by chromosomal rearrangements, e.g., cancer-associated chromosomal rearrangements. Detection of such rearrangements may lead to a diagnosis of a disease, e.g., cancer. It is now becoming apparent that splice variants are common and that mechanisms for controlling RNA splicing have evolved as a control mechanism for various physiological processes. The invention permits detection of expression of proteins encoded by such species, and correlation of the presence of such proteins with disease or abnormality. Examples of cancer-associated chromosomal rearrangements include: translocation t(16;21)(p11;q22) between genes FUS-ERG associated with myeloid leukemia and non-lymphocytic, acute leukemia (see Ichikawa H. et al. (1994) Cancer Res. 54(11):2865-8); translocation t(21;22)(q22;q12) between genes ERG-EWS associated with Ewing's sarcoma and neuroepithelioma (see Kaneko Y. et al. (1997) Genes Chromosomes Cancer 18(3):228-31); translocation t(14;18)(q32;q21) involving the bcl2 gene and associated with follicular lymphoma; and translocations juxtaposing the coding regions of the PAX3 gene on chromosome 2 and the FKHR gene on chromosome 13 associated with alveolar rhabdomyosarcoma (see Barr F. G. et al. (1996) Hum. Mol. Genet. 5:15-21).

For applications in environmental and industrial diagnostics the capture agents are designed such that they bind to one or more URS corresponding to a biowarfare agent (e.g., anthrax, small pox, cholera toxin) and/or one or more URS corresponding to other environmental toxins (Staphylococcus aureus a-toxin, Shiga toxin, cytotoxic necrotizing factor type 1, Escherichia coli heat-stable toxin, and botulinum and tetanus neurotoxins) or allergens. The capture agents may also be designed to bind to one or more URS corresponding to an infectious agent such as a bacterium, a prion, a parasite, or a URS corresponding to a virus (e.g., human immunodeficiency virus-1 (HIV-1), HIV-2, simian immunodeficiency virus (SIV), hepatitis C virus (HCV ), hepatitis B virus (HBV), Influenza, Foot and Mouth Disease virus, and Ebola virus).

B. High-Throughput Screening

Compositions containing the capture agents of the invention, e.g., microarrays, beads or chips enable the high-throughput screening of very large numbers of compounds to identify those compounds capable of interacting with a particular capture agent, or to detect molecules which compete for binding with the URSs. Microarrays are useful for screening large libraries of natural or synthetic compounds to identify competitors of natural or non-natural ligands for the capture agent, which may be of diagnostic, prognostic, therapeutic or scientific interest.

The use of microarray technology with the capture agents of the present invention enables comprehensive profiling of large numbers of proteins from normal and diseasedstate serum, cells, and tissues.

For example, once the microarray has been formed, it may be used for high-throughput drug discovery (e.g., screening libraries of compounds for their ability to bind to or modulate the activity of a target protein); for high-throughput target identification (e.g., correlating a protein with a disease process); for high-throughput target validation (e.g., manipulating a protein by, for example, mutagenesis and monitoring the effects of the manipulation on the protein or on other proteins); or in basic research (e.g., to study patterns of protein expression at, for example, key developmental or cell cycle time points or to study patterns of protein expression in response to various stimuli).

In one embodiment, the invention provides a method for identifying a test compound, e.g., a small molecule, that modulates the activity of a ligand of interest. According to this embodiment, a capture agent is exposed to a ligand and a test compound. The presence or the absence of binding between the capture agent and the ligand is then detected to determine the modulatory effect of the test compound on the ligand. In a preferred embodiment, a microarray of capture agents, that bind to ligands acting in the same cellular pathway, are used to profile the regulatory effect of a test compound on all these proteins in a parallel fashion.

C. Pharmacoproteomics

The capture agents or arrays comprising the capture agents of the present invention may also be used to study the relationship between a subject's protein expression profile and that subject's response to a foreign compound or drug. Differences in metabolism of therapeutics can lead to severe toxicity or therapeutic failure by altering the relation between dose and blood concentration of the pharmacologically active drug. Thus, use of the capture agents in the foregoing manner may aid a physician or clinician in determining whether to administer a pharmacologically active drug to a subject, as well as in tailoring the dosage and/or therapeutic regimen of treatment with the drug.

D. Protein Profiling

As indicated above, capture agents of the present invention enable the characterization of any biological state via protein profiling. The term “protein profile,” as used herein, includes the pattern of protein expression obtained for a given tissue or cell under a given set of conditions. Such conditions may include, but are not limited to, cellular growth, apoptosis, proliferation, differentiation, transformation, tumorigenesis, metastasis, and carcinogen exposure.

The capture agents of the present invention may also be used to compare the protein expression patterns of two cells or different populations of cells. Methods of comparing the protein expression of two cells or populations of cells are particularly useful for the understanding of biological processes. For example, using these methods, the protein expression patterns of identical cells or closely related cells exposed to different conditions can be compared. Most typically, the protein content of one cell or population of cells is compared to the protein content of a control cell or population of cells. As indicated above, one of the cells or populations of cells may be neoplastic and the other cell is not. In another embodiment, one of the two cells or populations of cells being assayed may be infected with a pathogen. Alternatively, one of the two cells or populations of cells has been exposed to a chemical, environmental, or thermal stress and the other cell or population of cells serves as a control. In a further embodiment, one of the cells or populations of cells may be exposed to a drug or a potential drug and its protein expression pattern compared to a control cell.

Such methods of assaying differential protein expression are useful in the identification and validation of new potential drug targets as well as for drug screening. For instance, the capture agents and the methods of the invention may be used to identify a protein which is overexpressed in tumor cells, but not in normal cells. This protein may be a target for drug intervention. Inhibitors to the action of the overexpressed protein can then be developed. Alternatively, antisense strategies to inhibit the overexpression may be developed. In another instance, the protein expression pattern of a cell, or population of cells, which has been exposed to a drug or potential drug can be compared to that of a cell, or population of cells, which has not been exposed to the drug. This comparison will provide insight as to whether the drug has had the desired effect on a target protein (drug efficacy) and whether other proteins of the cell, or population of cells, have also been affected (drug specificity).

E. Protein Sequencing, Purification and Characterization

The capture agents of the present invention may also be used in protein sequencing. Briefly, capture agents are raised that interact with a known combination of unique recognition sequences. Subsequently, a protein of interest is fragmented using the methods described herein to generate a collection of peptides and then the sample is allowed to interact with the capture agents. Based on the interaction pattern between the collection of peptides and the capture agents, the amino acid sequence of the collection of peptides may be deciphered. In a preferred embodiment, the capture agents are immobilized on an array in pre-determined positions that allow for easy determination of peptide-capture agent interactions. These sequencing methods would further allow the identification of amino acid polymorphisms, e.g., single amino acid polymorphisms, or mutations in a protein of interest.

In another embodiment, the capture agents of the present invention may also be used in protein purification. In this embodiment, the URS acts as a ligand/affinity tag and allows for affinity purification of a protein. A capture agent raised against a URS exposed on a surface of a protein may be coupled to a column of interest using art known techniques. The choice of a column will depend on the amino acid sequence of the capture agent and which end will be linked to the matrix. For example, if the amino-terminal end of the capture agent is to be linked to the matrix, matrices such as the Affigel (by Biorad) may be used. If a linkage via a cysteine residue is desired, an Epoxy-Sepharose-6B column (by Pharmacia) may be used. A sample containing the protein of interest may then be run through the column and the protein of interest may be eluted using art known techniques as described in, for example, J. Nilsson et al. (1997) “Affinity fusion strategies for detection, purification, and immobilization of recombinant proteins,” Protein Expression and Purification, 11:11-16, the contents of which are incorporated by reference. This embodiment of the invention also allows for the characterization of protein-protein interactions under native conditions, without the need to introduce artificial affinity tags in the protein(s) to be studied.

In yet another embodiment, the capture agents of the present invention may be used in protein characterization. Capture agents can be generated that differentiate between alternative forms of the same gene product, e.g., between proteins having different post-translational modifications (e.g., phosphorylated versus non-phosphorylated versions of the same protein or glycosylated versus non-glycosylated versions of the same protein) or between alternatively spliced gene products.

The utility of the invention is not limited to diagnosis. The system and methods described herein may also be useful for screening, making prognosis of disease outcomes, and providing treatment modality suggestion based on the profiling of the pathologic cells, prognosis of the outcome of a normal lesion and susceptibility of lesions to malignant transformation.

VII. Other Aspects of the Invention

In another aspect, the invention provides compositions comprising a plurality of isolated unique recognition sequences, wherein the unique recognition sequences are derived from at least 50%, 55%, 60%, 65%, 70%, 75%, 80%, 85%, 90% 95% or 100% of an organism's proteome. In one embodiment, each of the unique recognition sequences is derived from a different protein.

The present invention further provides methods for identifying and/or detecting a specific organism based on the organism's Proteome Epitope Tag. The methods include contacting a sample containing an organism of interest (e.g., a sample that has been fragmented using the methods described herein to generate a collection of peptides) with a collection of unique recognition sequences that characterize, and/or that are unique to, the proteome of the organism. In one embodiment, the collection of unique recognition sequences that comprise the Proteome Epitope Tag are immobilized on an array. These methods can be used to, for example, distinguish a specific bacterium or virus from a pool of other bacteria or viruses.

The unique recognition sequences of the present invention may also be used in a protein detection assay in which the unique recognition sequences are coupled to a plurality of capture agents that are attached to a support. The support is contacted with a sample of interest and, in the situation where the sample contains a protein that is recognized by one of the capture agents, the unique recognition sequence will be displaced from being bound to the capture agent. The unique recognition sequences may be labeled, e.g., fluorescently labeled, such that loss of signal from the support would indicate that the unique recognition sequence was displaced and that the sample contains a protein is recognized by one or more of the capture agents.

The unique recognition sequences of the present invention may also be used in therapeutic applications, e.g., to prevent or treat a disease in a subject. Specifically, the unique recognition sequences may be used as vaccines to elicit a desired immune response in a subject, such as an immune response against a tumor cell, an infectious agent or a parasitic agent. In this embodiment of the invention, a unique recognition sequence is selected that is unique to or is over-represented in, for example, a tissue of interest, an infectious agent of interest or a parasitic agent of interest. A unique recognition sequence is administered to a subject using art known techniques, such as those described in, for example, U.S. Pat. No. 5,925,362 and international publication Nos. WO 91/11465 and WO 95/24924, the contents of each of which are incorporated herein by reference. Briefly, the unique recognition sequence may be administered to a subject in a formulation designed to enhance the immune response. Suitable formulations include, but are not limited to, liposomes with or without additional adjuvants and/or cloning DNA encoding the unique recognition sequence into a viral or bacterial vector. The formulations, e.g., liposomal formulations, incorporating the unique recognition sequence may also include immune system adjuvants, including one or more of lipopolysaccharide (LPS), lipid A, muramyl dipeptide (MDP), glucan or certain cytokines, including interleukins, interferons, and colony stimulating factors, such as IL1, IL2, gamma interferon, and GM-CSF.

### EXAMPLES

This invention is further illustrated by the following examples which should not be construed as limiting. The contents of all references, patents and published patent applications cited throughout this application, as well as the Figures are hereby incorporated by reference.

### Example 1

Identification of Unique Recognition Equences within the Human Proteome

As any one of the total 20 amino acids could be at one specific position of a peptide, the total possible combination for a tetramer (a peptide containing 4 amino acid residues) is 20⁴; the total possible combination for a pentamer (a peptide containing 5 amino acid residues) is 20⁵ and the total possible combination for a hexamer (a peptide containing 6 amino acid residues) is 20⁶. In order to identify unique recognition sequences within the human proteome, each possible tetramer, pentamer or hexamer was searched against the human proteome (total number: 29,076; Source of human proteome: EBI Ensembl project release v 4.28.1 on Mar. 12, 2002, http://www.ensembl.org/Homo_sapiens/).

The results of this analysis, set forth below, indicate that using a pentamer as a unique recognition sequence, 80.6% (23,446 sequences) of the human proteome have their own unique recognition sequence(s). Using a hexamer as a unique recognition sequence, 89.7% of the human proteome have their own unique recognition sequence(s). In contrast, 5 when a tetramer is used as a unique recognition sequence, only 2.4% of the human proteome have their own unique recognition sequence(s).

Results and Data

2.1. Tetramer Analysis:

2.1.1. Sequence space:

| | | |
|---|---|---|
| Total number of human protein sequences | 29,076 | 100% |
| *Number of sequences with 1 or more unique | 684 | 2.4% |
| tetramer tag | | |
| Number of sequences with 0 unique tetramer tag | 28,392 | 97.6% |

*For these 684 sequences, average Tag/sequence: 1.1.

2.1.2. Tag space:

| | | |
|---|---|---|
| Total number of tetramers | 20⁴ = 160,000 | 100% |
| Tetramers found in 0 sequence | 393 | 0.2% |
| ^{#}Tetramers found in 1 sequence only | 745 | 0.5% |
| Tetramers found in more than 1 sequences | 158,862 | 99.3% |

^{#}These are signature tetra-peptides

2.2. Pentamer Analysis:

2.2.1. Sequence space:

| | | |
|---|---|---|
| Total number of human protein sequences | 29,076 | 100% |
| *Number of sequences with 1 or more unique | 23,446 | 80.6% |
| pentamer tag | | |
| Number of sequences with 0 unique pentamer tag | 5,630 | 19.4% |

*For these 23,446 sequences, Average Tag/sequence: 23.9

2.2.2. Tag space:

| | | |
|---|---|---|
| Total number of pentamers | 20⁵ = 3,200,000 | 100% |
| Pentamers found in 0 sequence | 955,007 | 29.8% |
| ^{#}Pentamers found in 1 sequence only | 560,309 | 17.5% |
| Pentamers found in more than 1 sequences | 1,684,684 | 52.6% |

^{#}These are signature penta-peptides

2.3. Hexamer Analysis:

2.3.1. Sequence space:

| | | |
|---|---|---|
| Total number of human protein sequences | 29,076 | 100% |
| *Number of sequences with 1 or more unique | 26,069 | 89.7% |
| hexamer tag | | |
| Number of sequences with 0 unique hexamer tag | 3,007 | 10.3% |

*For these 26069 sequences, Average Tag/sequence: 177

2.3.2. Tag space:

| | | |
|---|---|---|
| Total number of hexamers | 20⁶ = 64,000,000 | 100% |
| hexamers found in 0 sequence | 57,040,296 | 89.1% |
| ^{#}hexamers found in 1 sequence only | 4,609,172 | 7.2% |
| hexamers found in more than 1 sequences | 2,350,532 | 3.7% |

^{#}These are signature hexa-peptides.

Similar analysis in the human proteome was done for URS sequences of 7-10 amino acids in length, and the results are combinedly summarized in the table below:

| | Tagged | Tagged | Average |
|---|---|---|---|
| URS Length | Sequences | Sequences | URS (Number/ |
| (Amino Acids) | (Number) | (% of total - 29076) | Tagged Protein) |
| 4 | 684 | 2.35% | 3 |
| 5 | 23,446 | 80.64% | 24 |
| 6 | 26,069 | 89.66% | 177 |
| 7 | 26,184 | 90.05% | 254 |
| 8 | 26,216 | 90.16% | 268 |
| 9 | 26,238 | 90.24% | 272 |
| 10 | 26,250 | 90.28% | 275 |

### Example 2

Identification of Unique Recognition Sequences within all Bacterial Proteomes

In order to identify pentamer URSs that can be used to, for example, distinguish a specific bacterium from a pool of all other bacteria, each possible pentamer was searched against the NCBI database (http://www.ncbi.nlm.nih.gov/PMGifs/Genomes/eub_g.html, updated as of Apr. 10, 2002). The results from this analysis are set forth below.

Results and Data:

| Number of | Database ID | |
|---|---|---|
| unique | (NCBI | |
| pentamers | RefSeq ID) | Species Name |
| 6 | NC_000922 | Chlamydophila pneumoniae CWL029 |
| 37 | NC_002745 | Staphylococcus aureus N315 chromosome |
| 40 | NC_001733 | Methanococcus jannaschii small extra- |
| | | chromosomal element |
| 58 | NC_002491 | Chlamydophila pneumoniae J138 |
| 84 | NC_002179 | Chlamydophila pneumoniae AR39 |
| 135 | NC_000909 | Methanococcus jannaschii |
| 206 | NC_003305 | Agrobacterium tumefaciens str. C58 (U. |
| | | Washington) linear chromosome |
| 298 | NC_002758 | Staphylococcus aureus Mu50 chromosome |
| 356 | NC_002655 | Escherichia coli O157: H7 EDL933 |
| 386 | NC_003063 | Agrobacterium tumefaciens str. C58 (Cereon) |
| | | linear chromosome |
| 479 | NC_000962 | Mycobacterium tuberculosis |
| 481 | NC_002737 | Streptococcus pyogenes |
| 495 | NC_003304 | Agrobacterium tumefaciens str. C58 (U. |
| | | Washington) circular chromosome |
| 551 | NC_003098 | Streptococcus pneumonia R6 |
| 567 | NC_003485 | Streptococcus pyogenes MGAS8232 |
| 577 | NC_002695 | Escherichia coli O157 |
| 592 | NC_003028 | Streptococcus pneumonia TIGR4 |
| 702 | NC_003062 | Agrobacterium tumefaciens str. C58 (Cereon) |
| | | circular chromosome |
| 729 | NC_001263 | Deinococcus radiodurans chromosome 1 |
| 918 | NC_003116 | Neisseria meningitidis Z2491 |
| 924 | NC_000908 | Mycoplasma genitalium |
| 960 | NC_002755 | Mycobacterium tuberculosis CDC1551 |
| 977 | NC_003112 | Neisseria meningitidis MC58 |
| 979 | NC_000921 | Helicobacter pylori J99 |
| 1015 | NC_000915 | Helicobacter pylori 26695 |
| 1189 | NC_000963 | Rickettsia prowazekii |
| 1284 | NC_001318 | Borrelia burgdorferi chromosome |
| 1331 | NC_002771 | Mycoplasma pulmonis |
| 1426 | NC_000912 | Mycoplasma pneumoniae |
| 1431 | NC_002528 | Buchnera sp. APS |
| 1463 | NC_000868 | Pyrococcus abyssi |
| 1468 | NC_000117 | Chlamydia trachomatis |
| 1468 | NC_002162 | Ureaplasma urealyticum |
| 1478 | NC_003212 | Listeria innocua |
| 1553 | NC_003210 | Listeria monocytogenes |
| 1577 | NC_000961 | Pyrococcus horikoshii |
| 1630 | NC_002620 | Chlamydia muridarum |
| 1636 | NC_003103 | Rickettsia conorii Malish 7 |
| 1769 | NC_003198 | Salmonella typhi |
| 1794 | NC_000913 | Escherichia coli K12 |
| 1894 | NC_002689 | Thermoplasma volcanium |
| 1996 | NC_003413 | Pyrococcus furiosis |
| 2081 | NC_002578 | Thermoplasma acidophilum |
| 2106 | NC_003197 | Salmonella typhimurium LT2 |
| 2137 | NC_003317 | Brucella melitensis chromosome I |
| 2402 | NC_002677 | Mycobacterium leprae |
| 2735 | NC_000918 | Aquifex aeolicus |
| 2803 | NC_002505 | Vibrio cholerae chromosome 1 |
| 2900 | NC_000907 | Haemophilus influenzae |
| 3000 | NC_003318 | Brucella melitensis chromosome II |
| 3120 | NC_000854 | Aeropyrum pernix |
| 3229 | NC_002662 | Lactococcus lactis |
| 3287 | NC_002607 | Halobacterium sp. NRC-1 |
| 3298 | NC_003454 | Fusobacterium nucleatum |
| 3497 | NC_001732 | Methanococcus jannaschii large extra- |
| | | chromosomal element |
| 3548 | NC_002163 | Campylobacter jejuni |
| 3551 | NC_000853 | Thermotoga maritima |
| 3688 | NC_003106 | Sulfolobus tokodaii |
| 3775 | NC_002754 | Sulfolobus solfataricus |
| 3842 | NC_000919 | Treponema pallidum |
| 3921 | NC_003296 | Ralstonia solanacearum GMI1000 |
| 3940 | NC_000916 | Methanobacterium thermoautotrophicum |
| 4165 | NC_001264 | Deinococcus radiodurans chromosome 2 |
| 4271 | NC_003047 | Sinorhizobium meliloti 1021 chromosome |
| 4338 | NC_002663 | Pasteurella multocida |
| 4658 | NC_003364 | Pyrobaculum aerophilum |
| 5101 | NC_000917 | Archaeoglobus fulgidus |
| 5787 | NC_003366 | Clostridium perfringens |
| 5815 | NC_003450 | Corynebacterium glutamicum |
| 6520 | NC_002696 | Caulobacter crescentus |
| 6866 | NC_002506 | Vibrio cholerae chromosome 2 |
| 6891 | NC_003295 | Ralstonia solanacearum chromosome |
| 7078 | NC_002488 | Xylella fastidiosa chromosome |
| 8283 | NC_003143 | Yersinia pestis chromosome |
| 8320 | NC_000911 | Synechocystis PCC6803 |
| 8374 | NC_002570 | Bacillus halodurans |
| 8660 | NC_000964 | Bacillus subtilis |
| 8994 | NC_003030 | Clostridium acetobutylicum ATCC824 |
| 11725 | NC_003552 | Methanosarcina acetivorans |
| 12120 | NC_002516 | Pseudomonas aeruginosa |
| 12469 | NC_002678 | Mesorhizobium loti |
| 14022 | NC_003272 | Nostoc sp. PCC 7120 |

### Example 3

Identification of Specific Pentamer Unique Recognition Sequences

As indicated above, each possible tetramer, pentamer or hexamer was searched against the human proteome (total number: 29,076; Source of human proteome: EBI Ensembl project release 4.28.1 on Mar. 12, 2002, http://www.ensembl.org/Homo₁₃ sapiens/) to identify unique recognition sequences (URSs).

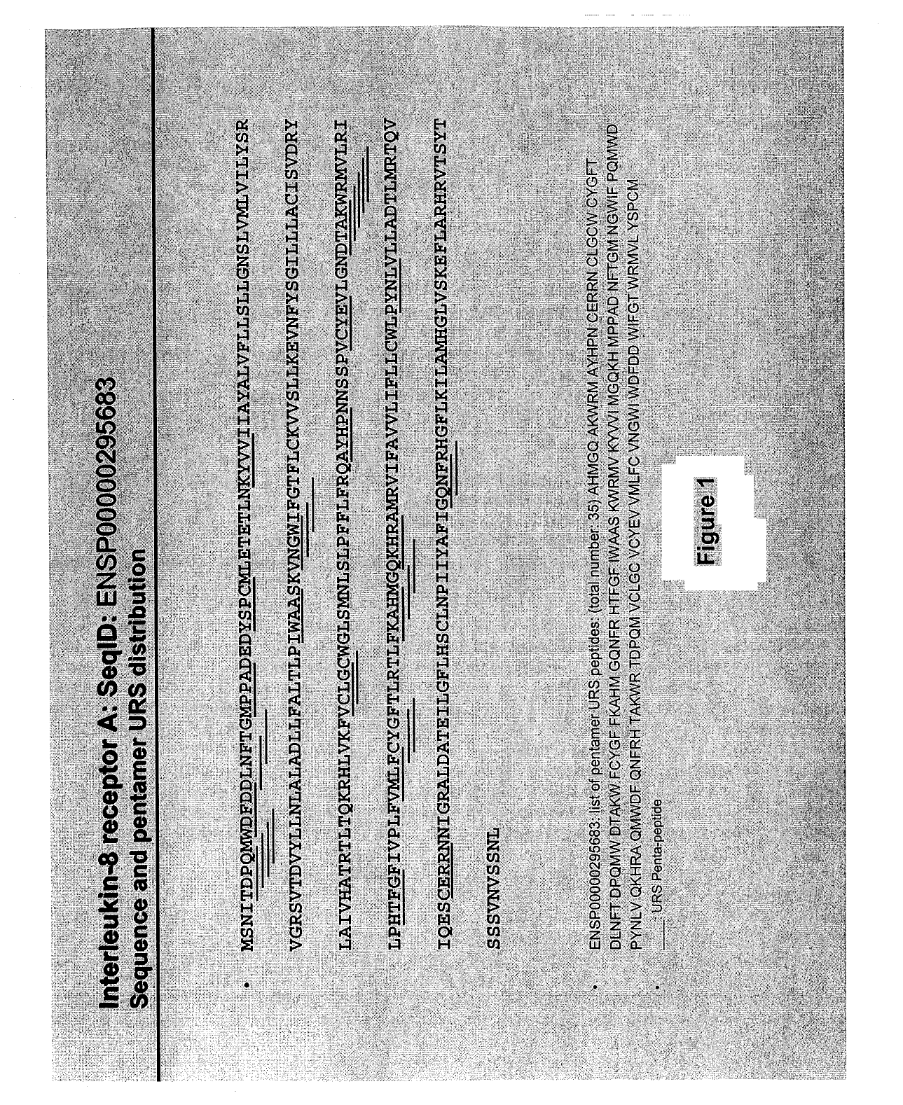

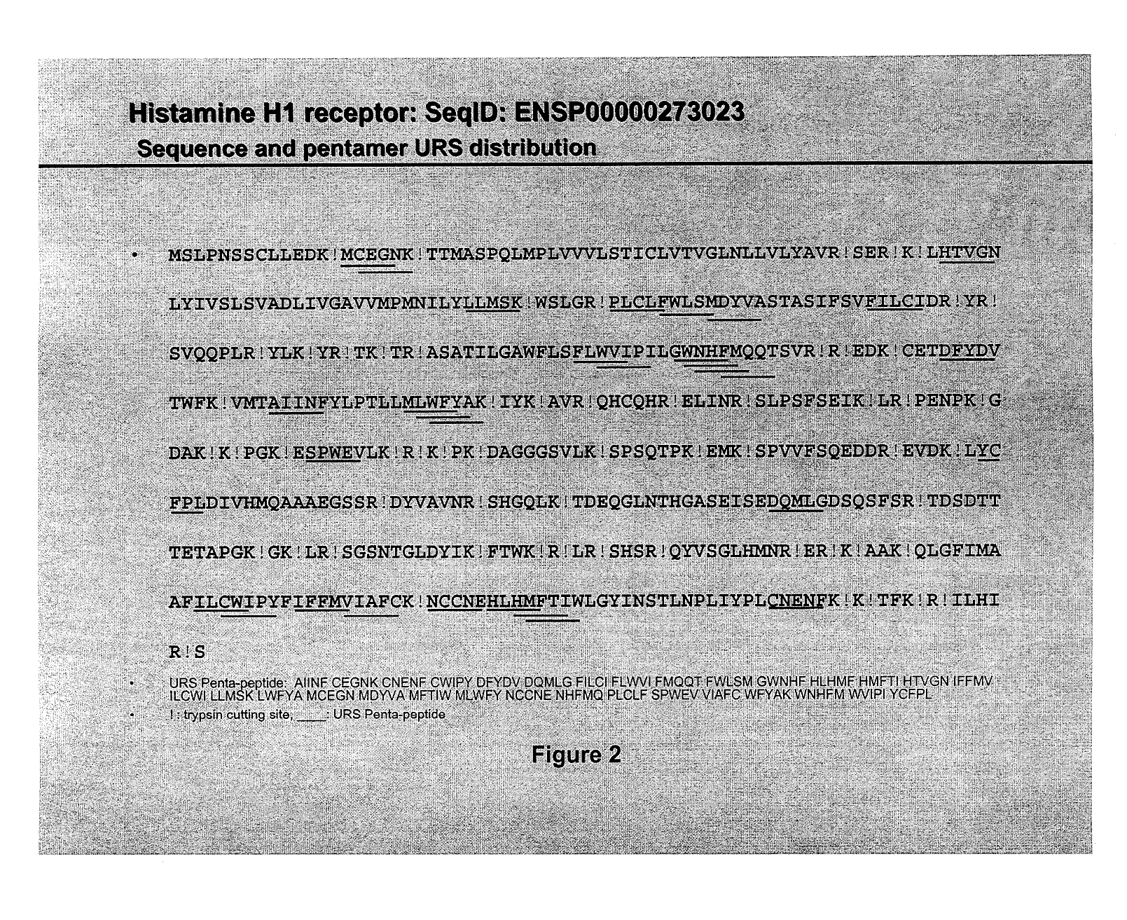

Based on the foregoing searches, specific URSs were identified for the majority of the human proteome. FIG. 1 depicts the pentamer unique recognition sequences that were identified within the sequence of the Interleukin-8 receptor A. FIG. 2 depicts the pentamer unique recognition sequences that were identified within the Histamine H1 receptor that are not destroyed by trypsin digestion. Further Examples of pentamer unique recognition sequences that were identified within the human proteome are set forth below.

| | Number | |
|---|---|---|
| | of | |
| | pentamer | |
| Sequence ID* | URSs | Pentamer URSs |
| ENSP00000000233 | 9 | AMPVS CATQG CFTVW ICFTV MPNAM PNAMP SRTWY |
| | | TWYVQ WYVQA |
| | | (SEQ ID NOs: 1-9) |
| ENSP00000000412 | 30 | CDFVC CGKEQ CWRTG DNFNP DNHCG FRVCR FYSCW |
| | | GMEQF HLAFW IFNGS IMLIY IYIFR KGMEQ KTCDL |
| | | MFPFY MISCN NETHI NWIML PFYSC QDCFY QFPHL |
| | | RESWQ SNWIM VMISC YDNHC YIYIF YKGGD YLFEM |
| | | YRGVG YSCWR |
| | | (SEQ ID NOs: 10-39) |
| ENSP00000000442 | 2 | ASNEC PASNE |
| | | (SEQ ID NOs: 40-41) |
| ENSP00000000449 | 9 | AQPWA ASTWR CLCLV FVICA LYCCP PRANR VNVLC |
| | | YAQLW YCCPV |
| | | (SEQ ID NOs: 42-50) |
| ENSP00000001008 | 20 | AIQRM AKPNE AMCHL AWDIA CQQRI ELKYE EMPMI |
| | | FVHYT HSIVY HYTGW LYANM MIGDR QKSNT SWEMN |
| | | SWLEY TEMPM WEMNS YAKPN YESSF YPNNK |
| | | (SEQ ID NOs: 51-70) |
| ENSP00000001146 | 32 | ATRDK CPCEG DKSCK DTHDT EWPRS FEVYQ FQIPK |
| | | FSGYR GCPCE GHLFE HDTAP IFSHE KEMTM KLQCT |
| | | KSCKL KYGNV LKHPT MGEHH MTMQE MYSIR NVFDP |
| | | QLWQL RGIQA RYLDC STEWP THDTA TRTFP VMYSI |
| | | VRTCL VSTEW WQLRW WSVMY |
| | | (SEQ ID NOs: 71-102) |
| ENSP00000001178 | 8 | ACKCF CKCFW FWLWY KCFWL LWYPH QKRRC WLWYP |
| | | WYPHF |
| | | (SEQ ID NOs: 103-110) |
| ENSP00000001380 | 26 | AMEQT APCTI AYMER CTIMK DGLCN EQTWR FRSYG |
| | | GMAYM GYHMP HIPNY KGRIP KLDMG MAYME MEQTW |
| | | MNKRE PGMNK QGYHM TMSPK TWRLD VEQGY VNDGL |
| | | WDQTR WRLDP YEAME YHMPC YNPCQ |
| | | (SEQ ID NOs: 111-136) |
| ENSP00000001567 | 137 | ATYYK CATYY CDNPY CEVVK CIKTD CINSR CKSPD |
| | | CKSSN CNELP CQENY CSESF CYERE CYHFG CYMGK |
| | | DFTWF DGWSA DIPIC DQTYP DREYH EEMHC EFDHN |
| | | EFNCS EHGWA EINYR EKIPC EMHCS ESNTG ESTCG |
| | | ESYAH EYHFG EYYCN FENAI FQYKC FTWFK GEWVA |
| | | GNVFE GWTND HGRKF HGTIN HGWAQ HPGYA HPPSC |
| | | HTVCI IHGVW IKHRT IMVCR INGRW IPCSQ IPVFM |
| | | IVCGY IYKCR IYKEN KCNMG KGEWV KIPCS KPCDY |
| | | KWSHP LPICY MENGW MGKWS MGYEY MIGHR NCSMA |
| | | NDFTW NEGYQ NETTC NGWSD NMGYE NQNHG NSVQC |
| | | NVFEY NYRDG NYREC PCDYP PEVNC PICYE PPQCE |
| | | PPYYY PQCVA PYIPN QCYHF QIQLC QYKVG RDTSC |
| | | REYHF RIKHR RKGEW RPCGH RVRYQ RWQSI SCDNP |
| | | SDQTY SFTMI SITCG SRWTG STGWI SVEFN SWSDQ |
| | | TAKCT TCIHG TCINS TCMEN TCYMG TMIGH TNDIP |
| | | TSTGW TWFKL TYKCF VAIDK VCGYN VEFNC VFEYG |
| | | VIMVC VNCSM VTYKC WDHIH WFKLN WIHTV WQSIP |
| | | WSDQT WTNDI YCNPR YHENM YHFGQ YKCFE YKCNM |
| | | YKCRP YKIEG YMGKW YNGWS YNQNH YPDIK YQCRN |
| | | YQYGE YSERG YWDHI YYKMD |
| | | (SEQ ID NOs: 137-274) |
| ENSP00000001585 | 25 | CVSKG EIIII GINYE GMKHA GWDLK HGMKH HHPKF |
| | | IEKCV IIMDA INYEI KGYVF MEMIV MIVRA NYTIG |
| | | QMEMI SHHPK TGSFR TRYKG VYGWD YGESK YGWDL |
| | | YIHGM YNERE YTIGE YVFQM |
| | | (SEQ ID NOs: 275-299) |
| ENSP00000002125 | 7 | GRYQR KNMGI MGERF PIKQH QRNAR RYQRN YDMLM |
| | | (SEQ ID NOs: 299-306) |
| ENSP00000002165 | 63 | AHSAT AKFFN CKWGW CMTID DKLSW DQAKF DVWYT |
| | | EYSWN FDQAK FEWFH FNANQ FWWYW FYTCS HKWEN |
| | | HPKAI HQMPC HTWRS IHQMP IPKYV IYETH KFFNA |
| | | KWENC KWGWA KWPTS LMNIG LPHKW MPCKW MRPQE |
| | | NANQW NCMTI NYPPS NYQPE PCKWG PDQYW PHKWE |
| | | QMGSW QYWNS RNRTD SCGGN SKHHE TCSDR THTWR |
| | | TIHQM TNDRW TPDVW TRFDP TVVTN VRGTV VVTND |
| | | WENCM WFDQA WFWWY WGSEY WGWAL WNWNA WRSQN |
| | | WWYWQ YEDFG YETHT YNPGH YSWNW YVEFM YYSLF |
| | | (SEQ ID NOs: 307-369) |
| ENSP00000002494 | 74 | AMNDA AKHGE AQWRN CVKLP CVQYK DAHKR DCVQY |
| | | DIEQR DMAER DPDKW DTANH EVSFM EYVID FEQYE |
| | | FFEQY FGDCV FMNET HEIYR HERFL HFDQT HKQWK |
| | | HKRAF HTAMN HWIQQ KHFDQ KMLNQ KQMTS KQYAQ |
| | | KRAFH KWERF LNGRW LPHWI MFATM MKFMN MKMEF |
| | | MLNQS MPQEG MYVKA NLPHW NTDAH NVLKH PHWIQ |
| | | PVMDA QADEM QENCK QHTAM QNYVS QWKDL QYAQA |
| | | RVPVM SFYDS SHERF TCDEM TDAHK TKLMP TVVRY |
| | | TYQIL VMDAQ VMKFM VPVMD VRYLF VSFMN WDRYG |
| | | WERFE WIIKY WIQQH WISTN WKDYT WKKHV YAQAD |
| | | YEVTY YGRRE YTDCV YVKAD |
| | | (SEQ ID NOs: 369-443) |
| ENSP00000002594 | 7 | CFKEN DGGFD FDLGD KLCFK KPMPN MPNPN PNPNH |
| | | (SEQ ID NOs: 444-450) |
| ENSP00000002596 | 36 | DRCLH EEHYS EHYSH ENEVH EYFHE FFDWE FHEPN |
| | | FSWPH FYNHM GRDRC GVAPN HEYFH HFFDW HIVDG |
| | | HKPYP HMQKH HMQNW HPQVD HVHMQ KGRAH KHKPY |
| | | KTPAY MQNWL NHMQK QKHKP QNWLR RVYSM SMNPS |
| | | SWPHQ TFDWH TQVFY WEEHY YCLRD YHVHM YNHMQ |
| | | YPSIE |
| | | (SEQ ID NOs: 451-486) |
| ENSP00000002829 | 60 | ADIRM AWPSF CLVNK CQAYG CTYVN DHDRM DPSFI |
| | | DRMYV GHCCL GIETH GYWRH HCCLV HDINR HDRMY |
| | | HQYCQ HRCQA IETHF IFYLE IHQYC IIHWA INFMR |
| | | IQPWN KMPYP KWLFQ LIIHW LIQPW MCTYV MPYPR |
| | | MRSHP NNFKH NPIRQ NSRWL NTTDY NYQWM PIRQC |
| | | PRNRR PVKTM PWNRT QDYIF QGYWR QTAMR RCQAY |
| | | RMVFN SKDYV SNANK TGAWP VGVTH VINFM VKWLF |
| | | WDGQA WPSFP WRHVP YAGVY YCQGY YNPMC YNSRW |
| | | YPLQR YQAVY YQWMP YWRHV |
| | | (SEQ ID NOs: 487-546) |

*The Sequence IDs used are the ones provided in http://www.ensembl.org/Homo_sapiens/

### Example 4

Idetection and Quantitation in a Complex Mixture of a Single a Peptide Sequence with Non-Overlapping URS Sequences Using Sandwich ELISA Assay

A fluorescence sandwich immunoassay for specific capture and quantitation of a targeted peptide in a complex peptide mixture is illustrated herein.

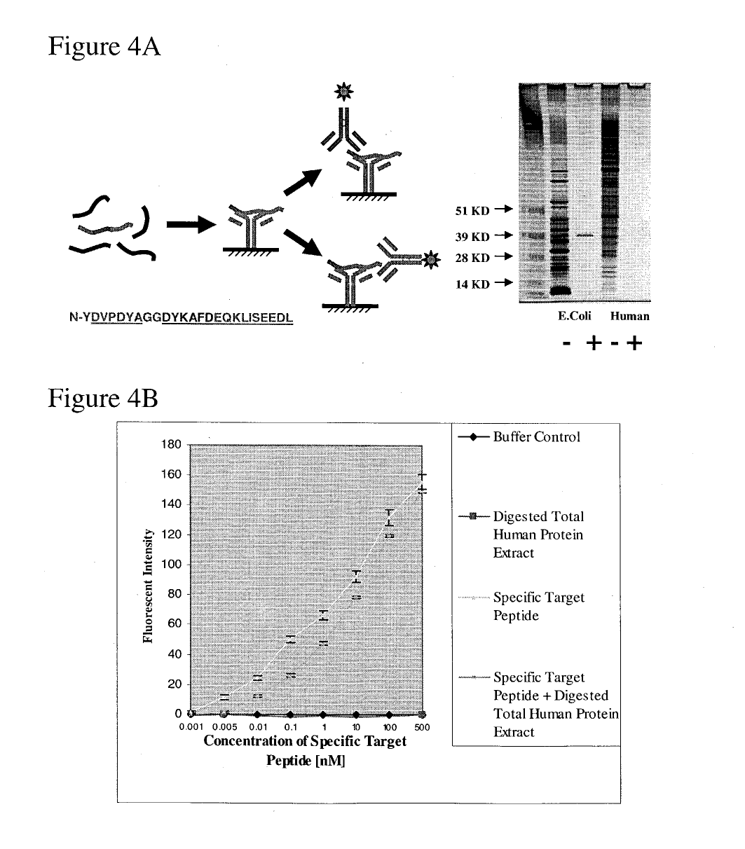

In the example shown here, a peptide consisting of three commonly used affinity epitope sequences (the HA tag, the FLAG tag and the MYC tag) is mixed with a large excess of unrelated peptides from digested human protein samples (FIG. 4a). The FLAG epitope in the middle of the target peptide is first captured here by the FLAG antibody, then the labeled antibody (either HA mAb or MYC mAb) is used to detect the second epitope. The final signal is detected by fluorescence readout from the secondary antibody. FIG. 4b shows that picomolar concentrations of HA-FLAG-MYC peptide was detected in the presence of a billion molar excess of digested unrelated proteins.

Equivalents

Those skilled in the art will recognize, or be able to ascertain using no more than routine experimentation, many equivalents to the specific embodiments of the invention described herein. Such equivalents are intended to be encompassed by the following claims.

We Claim:

1. A method of generating a set of capture agents for unambiguously identifying proteins in a sample, comprising:
computationally analyzing amino acid sequences for proteins expected to be present in a variegated sample of proteins, and generating data representative of amino acid sequences unique to each analyzed protein;
generating a set of reference reagents, each reference reagent independently including a unique amino acid sequence from one of said analyzed proteins;
generating a set of capture agents, each of which selectively binds a unique amino acid sequence of one of said reference reagents,
wherein collectively said set of capture agents can bind and unambiguously identifying the occurrence of a plurality of proteins present in said sample under conditions wherein said capture agents are contacted with said proteins, or fragments thereof, that have been rendered soluble in solution.

2. The method of claim 1, wherein said step of computationally analyzing amino acid sequences includes a Nearest-Neighbor Analysis that identifies unique amino acid sequences based on criteria that also include one or more of pI, charge, steric, solubility, hydrophobicity, polarity and solvent exposed area.

3. The method of claim 1, wherein said step of computationally analyzing amino acid sequences includes a solubility analysis that identifies unique amino acid sequences that are predicted to have at least a threshold solubility under a designated solution condition.

4. The method of claim 1, wherein said unique amino acid sequence is 5-30 amino acids long.

5. The method of claim 1, wherein said capture agents are antibodies, or antigen binding fragments thereof.

6. The method of claim 1, wherein said capture agents are selected from the group consisting of: nucleotides; nucleic acids; PNA (peptide nucleic acids); proteins; peptides; carbohydrates; artificial polymers; and small organic molecules.

7. The method of claim 1, wherein said capture agents are selected from the group consisting of aptamers, scaffolded peptides, and small organic molecules.

8. The method of claim 1, wherein said capture agents bind and unambiguously identifying proteins present in a solution of soluble proteins.

9. The method of claim 8, wherein said solution of soluble proteins is generated from denaturing and/or proteolysis of a sample proteins from a biological fluid.

10. The method of claim 9, wherein said solution of soluble proteins is generated from denaturing and/or proteolysis of a biological sample including cells.

11. The method of claim 1, wherein said set of capture agents are optimized for selectivity for said unique amino acid sequence under denaturing conditions.

12. The method of claim 1, including the further step of generating an array of said set of capture agents on the surface of beads or an array device in a manner that encodes the identity of a disposed capture agents.

13. The method of claim 12, wherein said array includes 100 or more different capture agents.

14. The method of claim 12, wherein said array device includes a diffractive grating surface.

15. The method of claim 12, wherein said capture agents are antibodies or antigen binding portions thereof, and said array is an arrayed ELISA.

16. The method of claim 12, wherein said array device is a surface plasmon resonance array.

17. The method of claim 12, wherein said beads are encoded as a virtual array.

18. The method of claim 1, including the further step of derivatizing said capture agents with a detectable label.

19. The method of claim 1 or 11, including the further step of packaging said capture agents with instructions for:
contacting the capture agents with a sample containing polypeptide analytes produced by denaturation and/or amide backbone cleavage; and
detecting interaction of said polypeptide analytes with said capture agents.

20. The method of claim 19, wherein the instructions further includes one or more of:
data for calibration procedures and preparation procedures, and statistical data on performance characteristics of the capture agents.

21. The method of claim 12, wherein the array has an greater statistical confidence, relative to an ELISA using antibodies generated against native proteins, for quantitating proteins in biological fluid or a solution of soluble proteins generated from denaturing and/or proteolysis of a biological sample including cells.

22. The method of claim 12, wherein the array has a regression coefficient (R2) of 0.95 or greater for a reference standard in biological fluid or a solution of soluble proteins generated from denaturing and/or proteolysis of a biological sample including cells.

23. The method of claim 12, wherein the array has a recovery rate of at least 50 percent.

24. The method of claim 12, wherein the array has an overall positive predictive value for occurrence of proteins in said sample of at least 90 percent.

25. The method of claim 12, wherein the array has an overall diagnostic sensitivity (DSN) for occurrence of proteins in said sample of 99 percent or higher.

26. The method of claim 12, wherein the array has an overall diagnostic specificity (DSP) for occurrence of proteins in said sample of 99 percent or higher.

27. A method for quantitating proteins in a biological sample, comprising
providing a plurality of different capture agents for detecting a plurality of different proteins in a test sample, which capture agents are provided as an addressable array, and each of which capture agents selectively interacts with a unique recognition sequence (URS);
contacting the array with a solution of polypeptide analytes produced by denaturation and/or cleavage of proteins from the test sample;
determining the identity and amount of proteins in the sample from the interaction of said polypeptide analytes with said capture agents,
wherein, for each capture agent, the method as a regression coefficient (R2) of 0.95 or greater.

28. The method of claim 27, wherein the array has a recovery rate of at least 50 percent.

29. A method for simultaneously detecting the presence of plural specific proteins in a multi-protein sample, the method comprising the steps of:
fragmenting proteins in the sample using a predetermined protocol to generate plural unique recognition sequences, the presence of which in said sample are indicative unambiguously of the presence of target proteins from which they are derived,
contacting at least a portion of the sample with plural capture agents which bind specifically to at least a portion of said unique recognition sequences under the conditions which obtain in the sample after the fragmentation, and,
detecting binding events as indicative of the presence of target proteins.

30. The method of claim 29, wherein the capture agents comprise binders for a set of unique recognition sequences which upon binding with a sample are indicative of the presence of a disease, physiologic state, or species.

31. A method for detecting the presence of one or more protein(s) in a sample, the method comprising:
(i) providing a solution of soluble peptide analytes produced by denaturations and/or cleavage of a plurality of sample proteins, and
(ii) optionally, labeling said collection of peptides by a detectable moiety;
(iii) contacting said solution with one or more capture agent(s), wherein each of said capture agent(s) is able to specifically recognize and interact with a unique recognition sequence (URS) of a reference protein; and,
(iv) detecting the binding between one or more of said capture agent(s) and said peptide analytes,
wherein the detection of binding between a capture agent and a peptide analyte indicates the presence of said reference protein in said plurality of sample proteins.

32. The method of claim 31, wherein said method is used in diagnosis, drug discovery or protein sequencing.

33. The method of claim 32, wherein said diagnosis is clinical diagnosis.

34. The method of claim 32, wherein said diagnosis is environmental diagnosis.

35. The method of claim 31, wherein said capture agents are selected from the group consisting of: nucleotides; nucleic acids; PNA (peptide nucleic acids); proteins; peptides; carbohydrates; artificial polymers; and small organic molecules.

36. The method of claim 31, wherein said capture agents are antibodies, or antigen binding fragments thereof.

37. The method of claim 36, wherein said capture agent is a full-length antibody, or a functional antibody fragment selected from: an Fab fragment, an F(ab′)₂ fragment, an Fd fragment, an Fv fragment, a dAb fragment, an isolated complementarity determining region (CDR), a single chain antibody (scFv), or derivative thereof.

38. The method of claim 36, wherein each of said capture agents is a single chain antibody.

39. The method of claim 35, wherein said capture agents are aptamers.

40. The method of claim 35, wherein said capture agents are scaffolded peptides.

41. The method of claim 35, wherein said capture agents are small organic molecules.

42. The method of claim 31, wherein said capture agents are immobilized on a solid support.

43. The method of claim 42, wherein said capture agents are arranged as an array on said solid support, with each capture agent occupying a distinct addressable location on said array.

44. The method of claim 42, wherein said capture agents are associated on the surface of encoded beads to form a virtual array of said capture agents.

45. The method of claim 43 or 44, wherein said array comprises at least 1,000 different capture agents bound to said support.

46. The method of claim 43 or 44, wherein said array comprises at least 10,000 different capture agents bound to said support.

47. The method of claim 43, wherein said capture agents are bound to said support at a density of 100 capture agents /cm².

48. The method of claim 31, wherein said soluble peptide analytes are produced by treatment of said sample proteins with a protease, a chemical agent, physical shearing, or sonication.

49. The method of claim 48, wherein said protease is trypsin, chymotrypsin, pepsin, papain, carboxypeptidase, calpain, subtilisin, gluc-C, endo lys-C or proteinase K.

50. The method of claim 48, wherein said soluble peptide analytes are produced by treatment of said sample proteins with a chemical agent.

51. The method of claim 50, wherein said chemical agent is cyanogen bromide.

52. The method of claim 31, wherein said protein sample is from a physiological, an environmental or an artificial source.

53. The method of claim 52, wherein said physiological source is body fluid selected from: saliva, mucous, sweat, whole blood, serum, urine, amniotic fluid, genital fluid, fecal material, marrow, plasma, spinal fluid, pericardial fluid, gastric fluid, abdominal fluid, peritoneal fluid, pleural fluid, synovial fluid, cyst fluid, cerebrospinal fluid, lung lavage fluid, lymphatic fluid, tears, prostatitc fluid, extraction from other body parts, or secretion from other glands.

54. The method of claim 52, wherein said protein sample is from supernatant, whole cell lysate, or cell fraction obtained by lysis and fractionation of cellular material, extract or fraction of cells obtained directly from a biological entity or cells grown in an artificial environment.

55. The method of claim 31, wherein said sample is obtained from human, mouse, rat, frog (Xenopus), fish (zebra fish), fly (Drosophila melanogaster), nematode (C. elegans), fission or budding yeast, or plant (Arabidopsis thaliana).

56. The method of claim 31, wherein said URS is a linear sequence.

57. The method of claim 31, wherein said URS is a non-contiguous sequence.

58. The method of claim 31, wherein said URS is 5-10 amino acids in length.

59. The method of claim 31, wherein said URS is 8 amino acids in length.

60. The method of claim 31, wherein said URS is selected from the group consisting of SEQ ID NOs: 1-546 or a sub-collection thereof.

61. The method of claim 31, for detection of a pathogen.

62. The method of claim 61, for detecting one or more toxins selected from anthrax toxin, small pox toxin, cholera toxin, Staphylococcus aureus a-toxin, Shiga toxin, cytotoxic necrotizing factor type 1, Escherichia coli heat-stable toxin, botulinum toxins, or tetanus neurotoxins.

63. The method of claim 31, wherein said soluble peptide analytes are produced by treatment of membrane bound proteins.

64. The method of claim 31, further comprising treating said sample proteins or said soluble peptide analytes to reduce post-translational modification of said soluble peptide analytes.

65. The method of claim 64, wherein said post-translational modification is phosphorylation, methylation, glycosylation, acetylation, or prenylation.

66. The method of claim 31, wherein said soluble peptide analytes are produced under conditions to preserve post-translational modification, and said capture agent(s) specifically interacts and discriminates between unmodified and post-translationally modified forms of said unique recognition sequence (URS) of a reference protein.

67. The method of claim 66, wherein said capture agent(s) specifically interact with and discriminate between post-translational modification of the reference protein selected from the group consisting of acetylation, amidation, deamidation, prenylation, formylation, glycosylation, hydroxylation, methylation, myristoylation, phosphorylation, ubiquitination, ribosylation and sulphation.

68. The method of claim 31, wherein step (2) is carried out, and step (4) is effectuated by detecting said detectable moiety on said soluble peptide analytes.

69. The method of claim 68, wherein said detectable moiety is a fluorescent label, a stainable dye, a chemilumninescent compound, a colloidal particle, a radioactive isotope, a near-infrared dye, a DNA dendrimer, a water-soluble quantum dot, a latex bead, a selenium particle, or a europium nanoparticle.

70. The method of claim 31, wherein step (2) is not carried out, and step (4) is effectuated by ELISA or immunoRCA.

71. The method of claim 31, wherein step (2) is not carried out, and step (4) is effectuated by mass spectrometry (MALDI-TOF), calorimetric resonant reflection using a SWS or SRVD biosensor, surface plasmon resonance (SPR), interferometry, gravimetry, ellipsometry, an evanascent wave device, resonance light scattering, reflectometry, a fluorescent polymer superquenching-based bioassay, or arrays of nanosensors comprising nanowires or nanotubes.

72. The method of claim 31, further comprising quantitating the amount of bound URS to each of said one or more capture agent(s).

73. The method of claim 31, wherein said capture agents are selected to detect a pattern of proteins in said protein sample that is indicative of a disease, physiologic state, or species.

74. The method of claim 31, wherein said sample proteins are treated with a pre-determined protocol which inhibits masking of a protein within said sample, such that upon fragmentation or denaturation of said protein, at least one URS whose concentration is directly proportional to the concentration of said protein in said sample is produced.

75. The method of claim 74, wherein said masking of said protein is caused by protein-protein complexation, protein degradation or denaturing, post-translational modification, or environmentally induced alteration in protein structure.

76. The method of claim 74, wherein binding of said capture agent to said unique recognition sequence is detected qualitatively.

77. The method of claim 74, wherein binding of said capture agent to said unique recognition sequence is detected quantitatively.

78. Apparatus for simultaneously detecting the presence of plural specific proteins in a multi-protein sample, the apparatus comprising:
a plurality of immobilized capture agents for contact with said sample, said capture agents including at least a subset of capture agents which each respectively bind specifically with a unique recognition sequence,
the presence of a particular unique recognition sequence being unambiguously indicative of the presence in said sample of a target protein from which it is derived,
each of said unique recognition sequences being generated reproducibly by a predetermined proteolytic and/or denaturation protocol performed on a said sample comprising said target protein, and
means for detecting binding between respective said capture agents and unique recognition sequences under the conditions which obtain in the sample after execution of the proteolytic and/or denaturation protocol.

79. The apparatus of claim 78, wherein said means for detecting binding events comprises means for detecting data indicative of the amount of bound unique recognition sequence thereby permitting assessment of the relative quantity of at least two target proteins in said sample.

80. A packaged protein detection array comprising:
(a) a plurality of different capture agents for detecting a plurality of different proteins in a sample, which capture agents are provided as an addressable array, and each of which capture agents selectively interacts with a unique recognition sequence (URS); and
(b) instructions for
contacting the addressable array with a sample containing polypeptide analytes produced by denaturation and/or cleavage of proteins at amide backbone positions, and
detecting interaction of said polypeptide analytes with said capture agent moieties.

81. The packaged protein detection array of claim 80, wherein the addressable array is an apparatus that comprises a plurality of said capture agents linked to a substrate in an array pattern of features.

82. The packaged protein detection array of claim 81, apparatus is coated with a layer to permit detection of binding of said capture agents with said polypeptide analytes by plasmon resonance detection.

83. The packaged protein detection array of claim 80, further including one or more labeled reference peptides including URS portions that bind to said capture agents, wherein said binding of said capture agents with said polypeptide analytes is detected by a competitive binding assay with said reference peptides.

84. The packaged protein detection array of claim 80, further including one or more antibodies which are immunoreactive with polypeptides including one of said URS, wherein said binding of said capture agents with said polypeptide analytes is detected by immunoassay.

85. The packaged protein detection array of claim 81, wherein the apparatus includes a grating comprised of a material having a high refractive index, a substrate layer that supports the two-dimensional grating, and said capture agents immobilized in discrete addressable locations on the surface of said grating opposite of the substrate layer such that, when said the apparatus is illuminated, a resonant grating effect is produced on reflected radiation in a manner dependent on the binding of a polypeptide analyte with a capture agent.

86. The packaged protein detection array of claim 80, wherein the addressable array is collection of beads, each of which comprises a discrete species of capture agent and one or more labels which identify the bead.

87. The packaged protein detection array of claim 80, wherein the plurality of different capture agents discriminate between unmodified and post-translationally modified forms of said unique recognition sequence (URS), and can unambiguously identify post-translationally modified forms of a protein in said sample.

88. The packaged protein detection array of claim 87, wherein said capture agents discriminate between post-translational modification of a protein selected from the group consisting of acetylation, amidation, deamidation, prenylation, formylation, glycosylation, hydroxylation, methylation, myristoylation, phosphorylation, ubiquitination, ribosylation and sulphation.

89. A business method for providing protein detection arrays, the method comprising:
(i) identifying one or more unique recognition sequence(s) (URSs) for each of one or more pre-determined protein(s);
(ii) generating one or more capture agent(s) for each of said URSs identified in (i), each of said capture agent(s) specifically bind one of said URSs for which said capture agent(s) is generated;
(iii) fabricating arrays of capture agent(s) generated in (ii), wherein each of said capture agents is bound to a different discrete region or address of said solid support;
(iv) packaging said arrays of capture agent(s) in (iv) for use in diagnostic and/or research experimentation.

90. The business method of claim 89, further comprising marketing said arrays of capture agent(s).

91. The business method of claim 89, further comprising distributing said arrays of capture agent(s).

92. A system for manufacturing and selling detection assays, comprising:
a computer-based customer order component for ordering at least one of a plurality of capture agent detection assays;
a detection assay production component for creating said capture agent detection assays;
a shipping component for shipping said capture agent detection assays; and
a billing component for billing a customer for said capture agent detection assays.

93. A composition comprising a plurality of capture agents, wherein said plurality of capture agents are, collectively, capable of specifically interacting with at least 25% of an organism's proteome, and wherein each of said capture agents is able to recognize and interact with only one unique recognition sequence within a protein of said proteome.

94. The composition of claim 93, wherein said capture agents are selected from the group consisting of: nucleotides; nucleic acids; PNA (peptide nucleic acids);
proteins; peptides; carbohydrates; artificial polymers; and small organic molecules.

95. The composition of claim 94, wherein said capture agents are antibodies, or antigen binding fragments thereof.

96. The composition of claim 95, wherein said capture agent is a full-length antibody, or a functional antibody fragment selected from: an Fab fragment, an F(ab′)₂ fragment, an Fd fragment, an Fv fragment, a dAb fragment, an isolated complementarity determining region (CDR), a single chain antibody (scFv), or derivative thereof.

97. The composition of claim 95, wherein each of said capture agents is a single chain antibody.

98. The composition of claim 94, wherein said capture agents are aptamers.

99. The composition of claim 94, wherein said capture agents are scaffolded peptides.

100. The composition of claim 94, wherein said capture agents are small organic molecules.

101. The composition of claim 93, wherein said organism is human.

102. The composition of claim 93, wherein said organism is a bacterial organism, a viral organism, or a plant organism.

103. An apparatus for simultaneously detecting the presence of a plurality of proteins in a sample, comprising:
(i) a solid support to which are bound a plurality of capture agents, wherein each of said capture agents is able to specifically recognize and interact with a unique recognition sequence (URS) within a protein; and
(ii) means for detecting the interaction of said capture agents with said corresponding unique recognition sequences.

104. The apparatus of claim 103, wherein said means for detecting the interaction of said capture agents with corresponding unique recognition sequences comprises means for quantitating the amount of said plurality of proteins in said sample.

105. Apparatus for simultaneously detecting the presence of a plural of specific proteins in a multi-protein sample, the apparatus comprising:
(a) a plurality of immobilized capture agents for contact with said sample, said capture agents including at least a subset of agents which respectively bind specifically with a unique recognition sequence, the presence of each said sequence being unambiguously indicative of the presence in said sample of a target protein from which it is derived, each said sequence being generated reproductively by a predetermined proteolytic protocol performed on a said sample comprising said target protein, and
(b) means for detecting binding events between respective said capture agents and unique recognition sequence.

106. The apparatus of claim 105, wherein said means for detecting binding events comprises means for detecting data indicative of the amount of bound unique recognition sequence thereby permitting assessment of the relative quantity of at least two target proteins in said sample.

107. A method for preparing an array of capture agents, comprising:
(a) providing a plurality of isolated unique recognition sequences (URSs), said plurality of URSs derived from proteins comprising at least 50% of an organism's proteome;
(b) generating a plurality of capture agents, each capable of specifically binding one of said plurality of URSs; and
(c) attaching said plurality of capture agents to a support having a plurality of
discrete regions, wherein each of said capture agents is bound to a different discrete region, thereby preparing an array of capture agents.

108. The method of claim 107, wherein each of said capture agents specifically recognize and binds a non-redundant URS.

109. A business method for generating arrays of capture agents for marketing in research and development, the method comprising:
(a) identifying one or more unique recognition sequence(s) (URSs) for each of one or more pre-determined protein(s);
(b) generating one or more capture agent(s) for each of said URSs identified in (1), each of said capture agent(s) specifically bind one of said URSs for which said capture agent(s) is generated;
(c) fabricating arrays of capture agent(s) generated in (2) on solid support, wherein each of said capture agents is bound to a different discrete region of said solid support;
(d) packaging said arrays of capture agent(s) in (3) for diagnosis and/or research use in commercial and/or academic laboratories.

110. The business method of claim 109, further comprising marketing said arrays of capture agent(s) in (c) or said packaged arrays of capture agent(s) in (d) to potential customers and/or distributors.

111. The business method of claim 109, further comprising distributing said arrays of capture agent(s) in (c) or said packaged arrays of capture agent(s) in (d) to customers and/or distributors.

112. A business method for generating arrays of capture agents for marketing in research and development, the method comprising:
(a) identifying one or more unique recognition sequence(s) (URSs) for each of one or more pre-determined protein(s);
(b) licensing to a third party the right to manufacture or use said one or more unique recognition sequence(s).

113. A method for quantitating various forms of post-translationally modified proteins in a biological sample, comprising
providing an addressable array having a multitude of features, each feature independently including a capture agent for detecting a protein in an unmodified or modified state, each of which capture agents selectively interacts with a unique recognition sequence (URS), and each feature providing discriminating binding to a particular modified and unmodified form of said URS occurring in a protein of the test sample;
contacting the array with a solution of soluble polypeptide analytes produced by denaturation and/or cleavage of proteins from the test sample, said soluble polypeptide analytes being produced under conditions to preserve post-translational modification; and
determining the identity and amount of post-translationally modified proteins in the sample from the interaction of said polypeptide analytes with said capture agents.

114. The method of claim 113, wherein said capture agent(s) specifically interacts and discriminates between a post-translational modification of the reference protein selected from the group consisting of acetylation, amidation, deamidation, prenylation, formylation, glycosylation, hydroxylation, methylation, myristoylation, phosphorylation, ubiquitination, ribosylation and sulphation.

115. A packaged protein detection array comprising
(a) an addressable array having a plurality of features, each feature independently including
a discrete type of capture agent that selectively interacts with a unique recognition sequence (URS) of an analyte protein under conditions in which the analyte protein is a soluble protein produced by proteolysis and/or denaturation, wherein said features of said array are disposed in a pattern or with a label to provide identity of interactions with capture agents can be ascertained;
(b) instructions for
contacting the addressable array with a sample containing polypeptide analytes produced by denaturation and/or cleavage of proteins at amide backbone positions,
detecting interaction of said polypeptide analytes with said capture agent moieties;
and determining the identity of polypeptide analytes, or native proteins from which they are derived, based on interaction with capture agent moieties.


SEQUENCE LISTING (614 sequences)

SEQ ID NO: 1 - protein, 5 aa, Human
AMPVS

SEQ ID NO: 2 - protein, 5 aa, Human
CATQG

SEQ ID NO: 3 - protein, 5 aa, Human
CFTVW

SEQ ID NO: 4 - protein, 5 aa, Human
ICFTV

SEQ ID NO: 5 - protein, 5 aa, Human
MPNAM

SEQ ID NO: 6 - protein, 5 aa, Human
PNAMP

SEQ ID NO: 7 - protein, 5 aa, Human
SRTWY

SEQ ID NO: 8 - protein, 5 aa, Human
TWYVQ

SEQ ID NO: 9 - protein, 5 aa, Human
WYVQA

SEQ ID NO: 10 - protein, 5 aa, Human
CDFVC

SEQ ID NO: 11 - protein, 5 aa, Human
CGKEQ

SEQ ID NO: 12 - protein, 5 aa, Human
CWRTG

SEQ ID NO: 13 - protein, 5 aa, Human
DNFNP

SEQ ID NO: 14 - protein, 5 aa, Human
DNHCG

SEQ ID NO: 15 - protein, 5 aa, Human
FRVCR

SEQ ID NO: 16 - protein, 5 aa, Human
FYSCW

SEQ ID NO: 17 - protein, 5 aa, Human
GMEQF

SEQ ID NO: 18 - protein, 5 aa, Human
HLAFW

SEQ ID NO: 19 - protein, 5 aa, Human
IFNGS

SEQ ID NO: 20 - protein, 5 aa, Human
IMLIY

SEQ ID NO: 21 - protein, 5 aa, Human
IYIFR

SEQ ID NO: 22 - protein, 5 aa, Human
KGMEQ

SEQ ID NO: 23 - protein, 5 aa, Human
KTCDL

SEQ ID NO: 24 - protein, 5 aa, Human
MFPFY

SEQ ID NO: 25 - protein, 5 aa, Human
MISCN

SEQ ID NO: 26 - protein, 5 aa, Human
NETHI

SEQ ID NO: 27 - protein, 5 aa, Human
NWIML

SEQ ID NO: 28 - protein, 5 aa, Human
PFYSC

SEQ ID NO: 29 - protein, 5 aa, Human
QDCFY

SEQ ID NO: 30 - protein, 5 aa, Human
QFPHL

SEQ ID NO: 31 - protein, 5 aa, Human
RESWQ

SEQ ID NO: 32 - protein, 5 aa, Human
SNWIM

SEQ ID NO: 33 - protein, 5 aa, Human
VMISC

SEQ ID NO: 34 - protein, 5 aa, Human
YDNHC

SEQ ID NO: 35 - protein, 5 aa, Human
YIYIF

SEQ ID NO: 36 - protein, 5 aa, Human
YKGGD

SEQ ID NO: 37 - protein, 5 aa, Human
YLFEM

SEQ ID NO: 38 - protein, 5 aa, Human
YRGVG

SEQ ID NO: 39 - protein, 5 aa, Human
YSCWR

SEQ ID NO: 40 - protein, 5 aa, Human
ASNEC

SEQ ID NO: 41 - protein, 5 aa, Human
PASNE

SEQ ID NO: 42 - protein, 5 aa, Human
AQPWA

SEQ ID NO: 43 - protein, 5 aa, Human
ASTWR

SEQ ID NO: 44 - protein, 5 aa, Human
CLCLV

SEQ ID NO: 45 - protein, 5 aa, Human
FVICA

SEQ ID NO: 46 - protein, 5 aa, Human
LYCCP

SEQ ID NO: 47 - protein, 5 aa, Human
PRANR

SEQ ID NO: 48 - protein, 5 aa, Human
VNVLC

SEQ ID NO: 49 - protein, 5 aa, Human
YAQLW

SEQ ID NO: 50 - protein, 5 aa, Human
YCCPV

SEQ ID NO: 51 - protein, 5 aa, Human
AIQRM

SEQ ID NO: 52 - protein, 5 aa, Human
AKPNE

SEQ ID NO: 53 - protein, 5 aa, Human
AMCHL

SEQ ID NO: 54 - protein, 5 aa, Human
AWDIA

SEQ ID NO: 55 - protein, 5 aa, Human
CQQRI

SEQ ID NO: 56 - protein, 5 aa, Human
ELKYE

SEQ ID NO: 57 - protein, 5 aa, Human
EMPMI

SEQ ID NO: 58 - protein, 5 aa, Human
FVHYT

SEQ ID NO: 59 - protein, 5 aa, Human
HSIVY

SEQ ID NO: 60 - protein, 5 aa, Human
HYTGW

SEQ ID NO: 61 - protein, 5 aa, Human
LYANM

SEQ ID NO: 62 - protein, 5 aa, Human
MIGDR

SEQ ID NO: 63 - protein, 5 aa, Human
QKSNT

SEQ ID NO: 64 - protein, 5 aa, Human
SWEMN

SEQ ID NO: 65 - protein, 5 aa, Human
SWLEY

SEQ ID NO: 66 - protein, 5 aa, Human
TEMPM

SEQ ID NO: 67 - protein, 5 aa, Human
WEMNS

SEQ ID NO: 68 - protein, 5 aa, Human
YAKPN

SEQ ID NO: 69 - protein, 5 aa, Human
YESSF

SEQ ID NO: 70 - protein, 5 aa, Human
YPNNK

SEQ ID NO: 71 - protein, 5 aa, Human
ATRDK

SEQ ID NO: 72 - protein, 5 aa, Human
CPCEG

SEQ ID NO: 73 - protein, 5 aa, Human
DKSCK

SEQ ID NO: 74 - protein, 5 aa, Human
DTHDT

SEQ ID NO: 75 - protein, 5 aa, Human
EWPRS

SEQ ID NO: 76 - protein, 5 aa, Human
FEVYQ

SEQ ID NO: 77 - protein, 5 aa, Human
FQIPK

SEQ ID NO: 78 - protein, 5 aa, Human
FSGYR

SEQ ID NO: 79 - protein, 5 aa, Human
GCPCE

SEQ ID NO: 80 - protein, 5 aa, Human
GHLFE

SEQ ID NO: 81 - protein, 5 aa, Human
HDTAP

SEQ ID NO: 82 - protein, 5 aa, Human
IFSHE

SEQ ID NO: 83 - protein, 5 aa, Human
KEMTM

SEQ ID NO: 84 - protein, 5 aa, Human
KLQCT

SEQ ID NO: 85 - protein, 5 aa, Human
KSCKL

SEQ ID NO: 86 - protein, 5 aa, Human
KYGNV

SEQ ID NO: 87 - protein, 5 aa, Human
LKHPT

SEQ ID NO: 88 - protein, 5 aa, Human
MGEHH

SEQ ID NO: 89 - protein, 5 aa, Human
MTMQE

SEQ ID NO: 90 - protein, 5 aa, Human
MYSIR

SEQ ID NO: 91 - protein, 5 aa, Human
NVFDP

SEQ ID NO: 92 - protein, 5 aa, Human
QLWQL

SEQ ID NO: 93 - protein, 5 aa, Human
RGIQA

SEQ ID NO: 94 - protein, 5 aa, Human
RYLDC

SEQ ID NO: 95 - protein, 5 aa, Human
STEWP

SEQ ID NO: 96 - protein, 5 aa, Human
THDTA

SEQ ID NO: 97 - protein, 5 aa, Human
TRTFP

SEQ ID NO: 98 - protein, 5 aa, Human
VMYSI

SEQ ID NO: 99 - protein, 5 aa, Human
VRTCL

SEQ ID NO: 100 - protein, 5 aa, Human
VSTEW

SEQ ID NO: 101 - protein, 5 aa, Human
WQLRW

SEQ ID NO: 102 - protein, 5 aa, Human
WSVMY

SEQ ID NO: 103 - protein, 5 aa, Human
ACKCF

SEQ ID NO: 104 - protein, 5 aa, Human
CKCFW

SEQ ID NO: 105 - protein, 5 aa, Human
FWLWY

SEQ ID NO: 106 - protein, 5 aa, Human
KCFWL

SEQ ID NO: 107 - protein, 5 aa, Human
LWYPH

SEQ ID NO: 108 - protein, 5 aa, Human
QKRRC

SEQ ID NO: 109 - protein, 5 aa, Human
WLWYP

SEQ ID NO: 110 - protein, 5 aa, Human
WYPHF

SEQ ID NO: 111 - protein, 5 aa, Human
AMEQT

SEQ ID NO: 112 - protein, 5 aa, Human
APCTI

SEQ ID NO: 113 - protein, 5 aa, Human
AYMER

SEQ ID NO: 114 - protein, 5 aa, Human
CTIMK

SEQ ID NO: 115 - protein, 5 aa, Human
DGLCN

SEQ ID NO: 116 - protein, 5 aa, Human
EQTWR

SEQ ID NO: 117 - protein, 5 aa, Human
FRSYG

SEQ ID NO: 118 - protein, 5 aa, Human
GMAYM

SEQ ID NO: 119 - protein, 5 aa, Human
GYHMP

SEQ ID NO: 120 - protein, 5 aa, Human
HIPNY

SEQ ID NO: 121 - protein, 5 aa, Human
KGRIP

SEQ ID NO: 122 - protein, 5 aa, Human
KLDMG

SEQ ID NO: 123 - protein, 5 aa, Human
MAYME

SEQ ID NO: 124 - protein, 5 aa, Human
MEQTW

SEQ ID NO: 125 - protein, 5 aa, Human
MNKRE

SEQ ID NO: 126 - protein, 5 aa, Human
PGMNK

SEQ ID NO: 127 - protein, 5 aa, Human
QGYHM

SEQ ID NO: 128 - protein, 5 aa, Human
TMSPK

SEQ ID NO: 129 - protein, 5 aa, Human
TWRLD

SEQ ID NO: 130 - protein, 5 aa, Human
VEQGY

SEQ ID NO: 131 - protein, 5 aa, Human
VNDGL

SEQ ID NO: 132 - protein, 5 aa, Human
WDQTR

SEQ ID NO: 133 - protein, 5 aa, Human
WRLDP

SEQ ID NO: 134 - protein, 5 aa, Human
YEAME

SEQ ID NO: 135 - protein, 5 aa, Human
YHMPC

SEQ ID NO: 136 - protein, 5 aa, Human
YNPCQ

SEQ ID NO: 137 - protein, 5 aa, Human
ATYYK

SEQ ID NO: 138 - protein, 5 aa, Human
CATYY

SEQ ID NO: 139 - protein, 5 aa, Human
CDNPY

SEQ ID NO: 140 - protein, 5 aa, Human
CEVVK

SEQ ID NO: 141 - protein, 5 aa, Human
CIKTD

SEQ ID NO: 142 - protein, 5 aa, Human
CINSR

SEQ ID NO: 143 - protein, 5 aa, Human
CKSPD

SEQ ID NO: 144 - protein, 5 aa, Human
CKSSN

SEQ ID NO: 145 - protein, 5 aa, Human
CNELP

SEQ ID NO: 146 - protein, 5 aa, Human
CQENY

SEQ ID NO: 147 - protein, 5 aa, Human
CSESF

SEQ ID NO: 148 - protein, 5 aa, Human
CYERE

SEQ ID NO: 149 - protein, 5 aa, Human
CYHFG

SEQ ID NO: 150 - protein, 5 aa, Human
CYMGK

SEQ ID NO: 151 - protein, 5 aa, Human
DFTWF

SEQ ID NO: 152 - protein, 5 aa, Human
DGWSA

SEQ ID NO: 153 - protein, 5 aa, Human
DIPIC

SEQ ID NO: 154 - protein, 5 aa, Human
DQTYP

SEQ ID NO: 155 - protein, 5 aa, Human
DREYH

SEQ ID NO: 156 - protein, 5 aa, Human
EEMHC

SEQ ID NO: 157 - protein, 5 aa, Human
EFDHN

SEQ ID NO: 158 - protein, 5 aa, Human
EFNCS

SEQ ID NO: 159 - protein, 5 aa, Human
EHGWA

SEQ ID NO: 160 - protein, 5 aa, Human
EINYR

SEQ ID NO: 161 - protein, 5 aa, Human
EKIPC

SEQ ID NO: 162 - protein, 5 aa, Human
EMHCS

SEQ ID NO: 163 - protein, 5 aa, Human
ESNTG

SEQ ID NO: 164 - protein, 5 aa, Human
ESTCG

SEQ ID NO: 165 - protein, 5 aa, Human
ESYAH

SEQ ID NO: 166 - protein, 5 aa, Human
EYHFG

SEQ ID NO: 167 - protein, 5 aa, Human
EYYCN

SEQ ID NO: 168 - protein, 5 aa, Human
FENAI

SEQ ID NO: 169 - protein, 5 aa, Human
FQYKC

SEQ ID NO: 170 - protein, 5 aa, Human
FTWFK

SEQ ID NO: 171 - protein, 5 aa, Human
GEWVA

SEQ ID NO: 172 - protein, 5 aa, Human
GNVFE

SEQ ID NO: 173 - protein, 5 aa, Human
GWTND

SEQ ID NO: 174 - protein, 5 aa, Human
HGRKF

SEQ ID NO: 175 - protein, 5 aa, Human
HGTIN

SEQ ID NO: 176 - protein, 5 aa, Human
HGWAQ

SEQ ID NO: 177 - protein, 5 aa, Human
HPGYA

SEQ ID NO: 178 - protein, 5 aa, Human
HPPSC

SEQ ID NO: 179 - protein, 5 aa, Human
HTVCI

SEQ ID NO: 180 - protein, 5 aa, Human
IHGVW

SEQ ID NO: 181 - protein, 5 aa, Human
IKHRT

SEQ ID NO: 182 - protein, 5 aa, Human
IMVCR

SEQ ID NO: 183 - protein, 5 aa, Human
INGRW

SEQ ID NO: 184 - protein, 5 aa, Human
IPCSQ

SEQ ID NO: 185 - protein, 5 aa, Human
IPVFM

SEQ ID NO: 186 - protein, 5 aa, Human
IVCGY

SEQ ID NO: 187 - protein, 5 aa, Human
IYKCR

SEQ ID NO: 188 - protein, 5 aa, Human
IYKEN

SEQ ID NO: 189 - protein, 5 aa, Human
KCNMG

SEQ ID NO: 190 - protein, 5 aa, Human
KGEWV

SEQ ID NO: 191 - protein, 5 aa, Human
KIPCS

SEQ ID NO: 192 - protein, 5 aa, Human
KPCDY

SEQ ID NO: 193 - protein, 5 aa, Human
KWSHP

SEQ ID NO: 194 - protein, 5 aa, Human
LPICY

SEQ ID NO: 195 - protein, 5 aa, Human
MENGW

SEQ ID NO: 196 - protein, 5 aa, Human
MGKWS

SEQ ID NO: 197 - protein, 5 aa, Human
MGYEY

SEQ ID NO: 198 - protein, 5 aa, Human
MIGHR

SEQ ID NO: 199 - protein, 5 aa, Human
NCSMA

SEQ ID NO: 200 - protein, 5 aa, Human
NDFTW

SEQ ID NO: 201 - protein, 5 aa, Human
NEGYQ

SEQ ID NO: 202 - protein, 5 aa, Human
NETTC

SEQ ID NO: 203 - protein, 5 aa, Human
NGWSD

SEQ ID NO: 204 - protein, 5 aa, Human
NMGYE

SEQ ID NO: 205 - protein, 5 aa, Human
NQNHG

SEQ ID NO: 206 - protein, 5 aa, Human
NSVQC

SEQ ID NO: 207 - protein, 5 aa, Human
NVFEY

SEQ ID NO: 208 - protein, 5 aa, Human
NYRDG

SEQ ID NO: 209 - protein, 5 aa, Human
NYREC

SEQ ID NO: 210 - protein, 5 aa, Human
PCDYP

SEQ ID NO: 211 - protein, 5 aa, Human
PEVNC

SEQ ID NO: 212 - protein, 5 aa, Human
PICYE

SEQ ID NO: 213 - protein, 5 aa, Human
PPQCE

SEQ ID NO: 214 - protein, 5 aa, Human
PPYYY

SEQ ID NO: 215 - protein, 5 aa, Human
PQCVA

SEQ ID NO: 216 - protein, 5 aa, Human
PYIPN

SEQ ID NO: 217 - protein, 5 aa, Human
QCYHF

SEQ ID NO: 218 - protein, 5 aa, Human
QIQLC

SEQ ID NO: 219 - protein, 5 aa, Human
QYKVG

SEQ ID NO: 220 - protein, 5 aa, Human
RDTSC

SEQ ID NO: 221 - protein, 5 aa, Human
REYHF

SEQ ID NO: 222 - protein, 5 aa, Human
RIKHR

SEQ ID NO: 223 - protein, 5 aa, Human
RKGEW

SEQ ID NO: 224 - protein, 5 aa, Human
RPCGH

SEQ ID NO: 225 - protein, 5 aa, Human
RVRYQ

SEQ ID NO: 226 - protein, 5 aa, Human
RWQSI

SEQ ID NO: 227 - protein, 5 aa, Human
SCDNP

SEQ ID NO: 228 - protein, 5 aa, Human
SDQTY

SEQ ID NO: 229 - protein, 5 aa, Human
SFTMI

SEQ ID NO: 230 - protein, 5 aa, Human
SITCG

SEQ ID NO: 231 - protein, 5 aa, Human
SRWTG

SEQ ID NO: 232 - protein, 5 aa, Human
STGWI

SEQ ID NO: 233 - protein, 5 aa, Human
SVEFN

SEQ ID NO: 234 - protein, 5 aa, Human
SWSDQ

SEQ ID NO: 235 - protein, 5 aa, Human
TAKCT

SEQ ID NO: 236 - protein, 5 aa, Human
TCIHG

SEQ ID NO: 237 - protein, 5 aa, Human
TCINS

SEQ ID NO: 238 - protein, 5 aa, Human
TCMEN

SEQ ID NO: 239 - protein, 5 aa, Human
TCYMG

SEQ ID NO: 240 - protein, 5 aa, Human
TMIGH

SEQ ID NO: 241 - protein, 5 aa, Human
TNDIP

SEQ ID NO: 242 - protein, 5 aa, Human
TSTGW

SEQ ID NO: 243 - protein, 5 aa, Human
TWFKL

SEQ ID NO: 244 - protein, 5 aa, Human
TYKCF

SEQ ID NO: 245 - protein, 5 aa, Human
VAIDK

SEQ ID NO: 246 - protein, 5 aa, Human
VCGYN

SEQ ID NO: 247 - protein, 5 aa, Human
VEFNC

SEQ ID NO: 248 - protein, 5 aa, Human
VFEYG

SEQ ID NO: 249 - protein, 5 aa, Human
VIMVC

SEQ ID NO: 250 - protein, 5 aa, Human
VNCSM

SEQ ID NO: 251 - protein, 5 aa, Human
VTYKC

SEQ ID NO: 252 - protein, 5 aa, Human
WDHIH

SEQ ID NO: 253 - protein, 5 aa, Human
WFKLN

SEQ ID NO: 254 - protein, 5 aa, Human
WIHTV

SEQ ID NO: 255 - protein, 5 aa, Human
WQSIP

SEQ ID NO: 256 - protein, 5 aa, Human
WSDQT

SEQ ID NO: 257 - protein, 5 aa, Human
WTNDI

SEQ ID NO: 258 - protein, 5 aa, Human
YCNPR

SEQ ID NO: 259 - protein, 5 aa, Human
YHENM

SEQ ID NO: 260 - protein, 5 aa, Human
YHFGQ

SEQ ID NO: 261 - protein, 5 aa, Human
YKCFE

SEQ ID NO: 262 - protein, 5 aa, Human
YKCNM

SEQ ID NO: 263 - protein, 5 aa, Human
YKCRP

SEQ ID NO: 264 - protein, 5 aa, Human
YKIEG

SEQ ID NO: 265 - protein, 5 aa, Human
YMGKW

SEQ ID NO: 266 - protein, 5 aa, Human
YNGWS

SEQ ID NO: 267 - protein, 5 aa, Human
YNQNH

SEQ ID NO: 268 - protein, 5 aa, Human
YPDIK

SEQ ID NO: 269 - protein, 5 aa, Human
YQCRN

SEQ ID NO: 270 - protein, 5 aa, Human
YQYGE

SEQ ID NO: 271 - protein, 5 aa, Human
YSERG

SEQ ID NO: 272 - protein, 5 aa, Human
YWDHI

SEQ ID NO: 273 - protein, 5 aa, Human
YYKMD

SEQ ID NO: 274 - protein, 5 aa, Human
CVSKG

SEQ ID NO: 275 - protein, 5 aa, Human
EIIII

SEQ ID NO: 276 - protein, 5 aa, Human
GINYE

SEQ ID NO: 277 - protein, 5 aa, Human
GMKHA

SEQ ID NO: 278 - protein, 5 aa, Human
GWDLK

SEQ ID NO: 279 - protein, 5 aa, Human
HGMKH

SEQ ID NO: 280 - protein, 5 aa, Human
HHPKF

SEQ ID NO: 281 - protein, 5 aa, Human
IEKCV

SEQ ID NO: 282 - protein, 5 aa, Human
IIMDA

SEQ ID NO: 283 - protein, 5 aa, Human
INYEI

SEQ ID NO: 284 - protein, 5 aa, Human
KGYVF

SEQ ID NO: 285 - protein, 5 aa, Human
MEMIV

SEQ ID NO: 286 - protein, 5 aa, Human
MIVRA

SEQ ID NO: 287 - protein, 5 aa, Human
NYTIG

SEQ ID NO: 288 - protein, 5 aa, Human
QMEMI

SEQ ID NO: 289 - protein, 5 aa, Human
SHHPK

SEQ ID NO: 290 - protein, 5 aa, Human
TGSFR

SEQ ID NO: 291 - protein, 5 aa, Human
TRYKG

SEQ ID NO: 292 - protein, 5 aa, Human
VYGWD

SEQ ID NO: 293 - protein, 5 aa, Human
YGESK

SEQ ID NO: 294 - protein, 5 aa, Human
YGWDL

SEQ ID NO: 295 - protein, 5 aa, Human
YIHGM

SEQ ID NO: 296 - protein, 5 aa, Human
YNERE

SEQ ID NO: 297 - protein, 5 aa, Human
YTIGE

SEQ ID NO: 298 - protein, 5 aa, Human
YVFQM

SEQ ID NO: 299 - protein, 5 aa, Human
GRYQR

SEQ ID NO: 300 - protein, 5 aa, Human
KNMGI

SEQ ID NO: 301 - protein, 5 aa, Human
MGERF

SEQ ID NO: 302 - protein, 5 aa, Human
PIKQH

SEQ ID NO: 303 - protein, 5 aa, Human
QRNAR

SEQ ID NO: 304 - protein, 5 aa, Human
RYQRN

SEQ ID NO: 305 - protein, 5 aa, Human
YDMLM

SEQ ID NO: 306 - protein, 5 aa, Human
AHSAT

SEQ ID NO: 307 - protein, 5 aa, Human
AKFFN

SEQ ID NO: 308 - protein, 5 aa, Human
CKWGW

SEQ ID NO: 309 - protein, 5 aa, Human
CMTID

SEQ ID NO: 310 - protein, 5 aa, Human
DKLSW

SEQ ID NO: 311 - protein, 5 aa, Human
DQAKF

SEQ ID NO: 312 - protein, 5 aa, Human
DVWYT

SEQ ID NO: 313 - protein, 5 aa, Human
EYSWN

SEQ ID NO: 314 - protein, 5 aa, Human
FDQAK

SEQ ID NO: 315 - protein, 5 aa, Human
FEWFH

SEQ ID NO: 316 - protein, 5 aa, Human
FNANQ

SEQ ID NO: 317 - protein, 5 aa, Human
FWWYW

SEQ ID NO: 318 - protein, 5 aa, Human
FYTCS

SEQ ID NO: 319 - protein, 5 aa, Human
HKWEN

SEQ ID NO: 320 - protein, 5 aa, Human
HPKAI

SEQ ID NO: 321 - protein, 5 aa, Human
HQMPC

SEQ ID NO: 322 - protein, 5 aa, Human
HTWRS

SEQ ID NO: 323 - protein, 5 aa, Human
IHQMP

SEQ ID NO: 324 - protein, 5 aa, Human
IPKYV

SEQ ID NO: 325 - protein, 5 aa, Human
IYETH

SEQ ID NO: 326 - protein, 5 aa, Human
KFFNA

SEQ ID NO: 327 - protein, 5 aa, Human
KWENC

SEQ ID NO: 328 - protein, 5 aa, Human
KWGWA

SEQ ID NO: 329 - protein, 5 aa, Human
KWPTS

SEQ ID NO: 330 - protein, 5 aa, Human
LMNIG

SEQ ID NO: 331 - protein, 5 aa, Human
LPHKW

SEQ ID NO: 332 - protein, 5 aa, Human
MPCKW

SEQ ID NO: 333 - protein, 5 aa, Human
MRPQE

SEQ ID NO: 334 - protein, 5 aa, Human
NANQW

SEQ ID NO: 335 - protein, 5 aa, Human
NCMTI

SEQ ID NO: 336 - protein, 5 aa, Human
NYPPS

SEQ ID NO: 337 - protein, 5 aa, Human
NYQPE

SEQ ID NO: 338 - protein, 5 aa, Human
PCKWG

SEQ ID NO: 339 - protein, 5 aa, Human
PDQYW

SEQ ID NO: 340 - protein, 5 aa, Human
PHKWE

SEQ ID NO: 341 - protein, 5 aa, Human
QMGSW

SEQ ID NO: 342 - protein, 5 aa, Human
QYWNS

SEQ ID NO: 343 - protein, 5 aa, Human
RNRTD

SEQ ID NO: 344 - protein, 5 aa, Human
SCGGN

SEQ ID NO: 345 - protein, 5 aa, Human
SKHHE

SEQ ID NO: 346 - protein, 5 aa, Human
TCSDR

SEQ ID NO: 347 - protein, 5 aa, Human
THTWR

SEQ ID NO: 348 - protein, 5 aa, Human
TIHQM

SEQ ID NO: 349 - protein, 5 aa, Human
TNDRW

SEQ ID NO: 350 - protein, 5 aa, Human
TPDVW

SEQ ID NO: 351 - protein, 5 aa, Human
TRFDP

SEQ ID NO: 352 - protein, 5 aa, Human
TVVTN

SEQ ID NO: 353 - protein, 5 aa, Human
VRGTV

SEQ ID NO: 354 - protein, 5 aa, Human
VVTND

SEQ ID NO: 355 - protein, 5 aa, Human
WENCM

SEQ ID NO: 356 - protein, 5 aa, Human
WFDQA

SEQ ID NO: 357 - protein, 5 aa, Human
WFWWY

SEQ ID NO: 358 - protein, 5 aa, Human
WGSEY

SEQ ID NO: 359 - protein, 5 aa, Human
WGWAL

SEQ ID NO: 360 - protein, 5 aa, Human
WNWNA

SEQ ID NO: 361 - protein, 5 aa, Human
WRSQN

SEQ ID NO: 362 - protein, 5 aa, Human
WWYWQ

SEQ ID NO: 363 - protein, 5 aa, Human
YEDFG

SEQ ID NO: 364 - protein, 5 aa, Human
YETHT

SEQ ID NO: 365 - protein, 5 aa, Human
YNPGH

SEQ ID NO: 366 - protein, 5 aa, Human
YSWNW

SEQ ID NO: 367 - protein, 5 aa, Human
YVEFM

SEQ ID NO: 368 - protein, 5 aa, Human
YYSLF

SEQ ID NO: 369 - protein, 5 aa, Human
AMNDA

SEQ ID NO: 370 - protein, 5 aa, Human
ANHGE

SEQ ID NO: 371 - protein, 5 aa, Human
AQWRN

SEQ ID NO: 372 - protein, 5 aa, Human
CVKLP

SEQ ID NO: 373 - protein, 5 aa, Human
CVQYK

SEQ ID NO: 374 - protein, 5 aa, Human
DAHKR

SEQ ID NO: 375 - protein, 5 aa, Human
DCVQY

SEQ ID NO: 376 - protein, 5 aa, Human
DIEQR

SEQ ID NO: 377 - protein, 5 aa, Human
DMAER

SEQ ID NO: 378 - protein, 5 aa, Human
DPDKW

SEQ ID NO: 379 - protein, 5 aa, Human
DTANH

SEQ ID NO: 380 - protein, 5 aa, Human
EVSFM

SEQ ID NO: 381 - protein, 5 aa, Human
EYVID

SEQ ID NO: 382 - protein, 5 aa, Human
FEQYE

SEQ ID NO: 383 - protein, 5 aa, Human
FFEQY

SEQ ID NO: 384 - protein, 5 aa, Human
FGDCV

SEQ ID NO: 385 - protein, 5 aa, Human
FMNET

SEQ ID NO: 386 - protein, 5 aa, Human
HEIYR

SEQ ID NO: 387 - protein, 5 aa, Human
HERFL

SEQ ID NO: 388 - protein, 5 aa, Human
HFDQT

SEQ ID NO: 389 - protein, 5 aa, Human
HKQWK

SEQ ID NO: 390 - protein, 5 aa, Human
HKRAF

SEQ ID NO: 391 - protein, 5 aa, Human
HTAMN

SEQ ID NO: 392 - protein, 5 aa, Human
HWIQQ

SEQ ID NO: 393 - protein, 5 aa, Human
KHFDQ

SEQ ID NO: 394 - protein, 5 aa, Human
KMLNQ

SEQ ID NO: 395 - protein, 5 aa, Human
KQMTS

SEQ ID NO: 396 - protein, 5 aa, Human
KQYAQ

SEQ ID NO: 397 - protein, 5 aa, Human
KRAFH

SEQ ID NO: 398 - protein, 5 aa, Human
KWERF

SEQ ID NO: 399 - protein, 5 aa, Human
LNGRW

SEQ ID NO: 400 - protein, 5 aa, Human
LPHWI

SEQ ID NO: 401 - protein, 5 aa, Human
MFATM

SEQ ID NO: 402 - protein, 5 aa, Human
MKFMN

SEQ ID NO: 403 - protein, 5 aa, Human
MKMEF

SEQ ID NO: 404 - protein, 5 aa, Human
MLNQS

SEQ ID NO: 405 - protein, 5 aa, Human
MPQEG

SEQ ID NO: 406 - protein, 5 aa, Human
MYVKA

SEQ ID NO: 407 - protein, 5 aa, Human
NLPHW

SEQ ID NO: 408 - protein, 5 aa, Human
NTDAH

SEQ ID NO: 409 - protein, 5 aa, Human
NVLKH

SEQ ID NO: 410 - protein, 5 aa, Human
PHWIQ

SEQ ID NO: 411 - protein, 5 aa, Human
PVMDA

SEQ ID NO: 412 - protein, 5 aa, Human
QADEM

SEQ ID NO: 413 - protein, 5 aa, Human
QENCK

SEQ ID NO: 414 - protein, 5 aa, Human
QHTAM

SEQ ID NO: 415 - protein, 5 aa, Human
QNYVS

SEQ ID NO: 416 - protein, 5 aa, Human
QWKDL

SEQ ID NO: 417 - protein, 5 aa, Human
QYAQA

SEQ ID NO: 418 - protein, 5 aa, Human
RVPVM

SEQ ID NO: 419 - protein, 5 aa, Human
SFYDS

SEQ ID NO: 420 - protein, 5 aa, Human
SHERF

SEQ ID NO: 421 - protein, 5 aa, Human
TCDEM

SEQ ID NO: 422 - protein, 5 aa, Human
TDAHK

SEQ ID NO: 423 - protein, 5 aa, Human
TKLMP

SEQ ID NO: 424 - protein, 5 aa, Human
TVVRY

SEQ ID NO: 425 - protein, 5 aa, Human
TYQIL

SEQ ID NO: 426 - protein, 5 aa, Human
VMDAQ

SEQ ID NO: 427 - protein, 5 aa, Human
VMKFM

SEQ ID NO: 428 - protein, 5 aa, Human
VPVMD

SEQ ID NO: 429 - protein, 5 aa, Human
VRYLF

SEQ ID NO: 430 - protein, 5 aa, Human
VSFMN

SEQ ID NO: 431 - protein, 5 aa, Human
WDRYG

SEQ ID NO: 432 - protein, 5 aa, Human
WERFE

SEQ ID NO: 433 - protein, 5 aa, Human
WIIKY

SEQ ID NO: 434 - protein, 5 aa, Human
WIQQH

SEQ ID NO: 435 - protein, 5 aa, Human
WISTN

SEQ ID NO: 436 - protein, 5 aa, Human
WKDYT

SEQ ID NO: 437 - protein, 5 aa, Human
WKKHV

SEQ ID NO: 438 - protein, 5 aa, Human
YAQAD

SEQ ID NO: 439 - protein, 5 aa, Human
YEVTY

SEQ ID NO: 440 - protein, 5 aa, Human
YGRRE

SEQ ID NO: 441 - protein, 5 aa, Human
YTDCV

SEQ ID NO: 442 - protein, 5 aa, Human
YVKAD

SEQ ID NO: 443 - protein, 5 aa, Human
CFKEN

SEQ ID NO: 444 - protein, 5 aa, Human
DGGFD

SEQ ID NO: 445 - protein, 5 aa, Human
FDLGD

SEQ ID NO: 446 - protein, 5 aa, Human
KLCFK

SEQ ID NO: 447 - protein, 5 aa, Human
KPMPN

SEQ ID NO: 448 - protein, 5 aa, Human
MPNPN

SEQ ID NO: 449 - protein, 5 aa, Human
PNPNH

SEQ ID NO: 450 - protein, 5 aa, Human
DRCLH

SEQ ID NO: 451 - protein, 5 aa, Human
EEHYS

SEQ ID NO: 452 - protein, 5 aa, Human
EHYSH

SEQ ID NO: 453 - protein, 5 aa, Human
ENEVH

SEQ ID NO: 454 - protein, 5 aa, Human
EYFHE

SEQ ID NO: 455 - protein, 5 aa, Human
FFDWE

SEQ ID NO: 456 - protein, 5 aa, Human
FHEPN

SEQ ID NO: 457 - protein, 5 aa, Human
FSWPH

SEQ ID NO: 458 - protein, 5 aa, Human
FYNHM

SEQ ID NO: 459 - protein, 5 aa, Human
GRDRC

SEQ ID NO: 460 - protein, 5 aa, Human
GVAPN

SEQ ID NO: 461 - protein, 5 aa, Human
HEYFH

SEQ ID NO: 462 - protein, 5 aa, Human
HFFDW

SEQ ID NO: 463 - protein, 5 aa, Human
HIVDG

SEQ ID NO: 464 - protein, 5 aa, Human
HKPYP

SEQ ID NO: 465 - protein, 5 aa, Human
HMQKH

SEQ ID NO: 466 - protein, 5 aa, Human
HMQNW

SEQ ID NO: 467 - protein, 5 aa, Human
HPQVD

SEQ ID NO: 468 - protein, 5 aa, Human
HVHMQ

SEQ ID NO: 469 - protein, 5 aa, Human
KGRAH

SEQ ID NO: 470 - protein, 5 aa, Human
KHKPY

SEQ ID NO: 471 - protein, 5 aa, Human
KTPAY

SEQ ID NO: 472 - protein, 5 aa, Human
MQNWL

SEQ ID NO: 473 - protein, 5 aa, Human
NHMQK

SEQ ID NO: 474 - protein, 5 aa, Human
QKHKP

SEQ ID NO: 475 - protein, 5 aa, Human
QNWLR

SEQ ID NO: 476 - protein, 5 aa, Human
RVYSM

SEQ ID NO: 477 - protein, 5 aa, Human
SMNPS

SEQ ID NO: 478 - protein, 5 aa, Human
SWPHQ

SEQ ID NO: 479 - protein, 5 aa, Human
TFDWH

SEQ ID NO: 480 - protein, 5 aa, Human
TQVFY

SEQ ID NO: 481 - protein, 5 aa, Human
WEEHY

SEQ ID NO: 482 - protein, 5 aa, Human
YCLRD

SEQ ID NO: 483 - protein, 5 aa, Human
YHVHM

SEQ ID NO: 484 - protein, 5 aa, Human
YNHMQ

SEQ ID NO: 485 - protein, 5 aa, Human
YPSIE

SEQ ID NO: 486 - protein, 5 aa, Human
ADIRM

SEQ ID NO: 487 - protein, 5 aa, Human
AWPSF

SEQ ID NO: 488 - protein, 5 aa, Human
CLVNK

SEQ ID NO: 489 - protein, 5 aa, Human
CQAYG

SEQ ID NO: 490 - protein, 5 aa, Human
CTYVN

SEQ ID NO: 491 - protein, 5 aa, Human
DHDRM

SEQ ID NO: 492 - protein, 5 aa, Human
DPSFI

SEQ ID NO: 493 - protein, 5 aa, Human
DRMYV

SEQ ID NO: 494 - protein, 5 aa, Human
GHCCL

SEQ ID NO: 495 - protein, 5 aa, Human
GIETH

SEQ ID NO: 496 - protein, 5 aa, Human
GYWRH

SEQ ID NO: 497 - protein, 5 aa, Human
HCCLV

SEQ ID NO: 498 - protein, 5 aa, Human
HDINR

SEQ ID NO: 499 - protein, 5 aa, Human
HDRMY

SEQ ID NO: 500 - protein, 5 aa, Human
HQYCQ

SEQ ID NO: 501 - protein, 5 aa, Human
HRCQA

SEQ ID NO: 502 - protein, 5 aa, Human
IETHF

SEQ ID NO: 503 - protein, 5 aa, Human
IFYLE

SEQ ID NO: 504 - protein, 5 aa, Human
IHQYC

SEQ ID NO: 505 - protein, 5 aa, Human
IIHWA

SEQ ID NO: 506 - protein, 5 aa, Human
INFMR

SEQ ID NO: 507 - protein, 5 aa, Human
IQPWN

SEQ ID NO: 508 - protein, 5 aa, Human
KMPYP

SEQ ID NO: 509 - protein, 5 aa, Human
KWLFQ

SEQ ID NO: 510 - protein, 5 aa, Human
LIIHW

SEQ ID NO: 511 - protein, 5 aa, Human
LIQPW

SEQ ID NO: 512 - protein, 5 aa, Human
MCTYV

SEQ ID NO: 513 - protein, 5 aa, Human
MPYPR

SEQ ID NO: 514 - protein, 5 aa, Human
MRSHP

SEQ ID NO: 515 - protein, 5 aa, Human
NNFKH

SEQ ID NO: 516 - protein, 5 aa, Human
NPIRQ

SEQ ID NO: 517 - protein, 5 aa, Human
NSRWL

SEQ ID NO: 518 - protein, 5 aa, Human
NTTDY

SEQ ID NO: 519 - protein, 5 aa, Human
NYQWM

SEQ ID NO: 520 - protein, 5 aa, Human
PIRQC

SEQ ID NO: 521 - protein, 5 aa, Human
PRNRR

SEQ ID NO: 522 - protein, 5 aa, Human
PVKTM

SEQ ID NO: 523 - protein, 5 aa, Human
PWNRT

SEQ ID NO: 524 - protein, 5 aa, Human
QDYIF

SEQ ID NO: 525 - protein, 5 aa, Human
QGYWR

SEQ ID NO: 526 - protein, 5 aa, Human
QTAMR

SEQ ID NO: 527 - protein, 5 aa, Human
RCQAY

SEQ ID NO: 528 - protein, 5 aa, Human
RMVFN

SEQ ID NO: 529 - protein, 5 aa, Human
SKDYV

SEQ ID NO: 530 - protein, 5 aa, Human
SNANK

SEQ ID NO: 531 - protein, 5 aa, Human
TGAWP

SEQ ID NO: 532 - protein, 5 aa, Human
VGVTH

SEQ ID NO: 533 - protein, 5 aa, Human
VINFM

SEQ ID NO: 534 - protein, 5 aa, Human
VKWLF

SEQ ID NO: 535 - protein, 5 aa, Human
WDGQA

SEQ ID NO: 536 - protein, 5 aa, Human
WPSFP

SEQ ID NO: 537 - protein, 5 aa, Human
WRHVP

SEQ ID NO: 538 - protein, 5 aa, Human
YAGVY

SEQ ID NO: 539 - protein, 5 aa, Human
YCQGY

SEQ ID NO: 540 - protein, 5 aa, Human
YNPMC

SEQ ID NO: 541 - protein, 5 aa, Human
YNSRW

SEQ ID NO: 542 - protein, 5 aa, Human
YPLQR

SEQ ID NO: 543 - protein, 5 aa, Human
YQAVY

SEQ ID NO: 544 - protein, 5 aa, Human
YQWMP

SEQ ID NO: 545 - protein, 5 aa, Human
YWRHV

SEQ ID NO: 546 - protein, 350 aa, Human
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 547 - protein, 5 aa, Human
AHMGQ

SEQ ID NO: 548 - protein, 5 aa, Human
AKWRM

SEQ ID NO: 549 - protein, 5 aa, Human
AYHPN

SEQ ID NO: 550 - protein, 5 aa, Human
CERRN

SEQ ID NO: 551 - protein, 5 aa, Human
CLGCW

SEQ ID NO: 552 - protein, 5 aa, Human
CYGFT

SEQ ID NO: 553 - protein, 5 aa, Human
DLNFT

SEQ ID NO: 554 - protein, 5 aa, Human
DPQMW

SEQ ID NO: 555 - protein, 5 aa, Human
DTAKW

SEQ ID NO: 556 - protein, 5 aa, Human
FCYGF

SEQ ID NO: 557 - protein, 5 aa, Human
FKAHM

SEQ ID NO: 558 - protein, 5 aa, Human
GQNFR

SEQ ID NO: 559 - protein, 5 aa, Human
HTFGF

SEQ ID NO: 560 - protein, 5 aa, Human
IWAAS

SEQ ID NO: 561 - protein, 5 aa, Human
KWRMV

SEQ ID NO: 562 - protein, 5 aa, Human
KYVVI

SEQ ID NO: 563 - protein, 5 aa, Human
MGQKH

SEQ ID NO: 564 - protein, 5 aa, Human
MPPAD

SEQ ID NO: 565 - protein, 5 aa, Human
NFTGM

SEQ ID NO: 566 - protein, 5 aa, Human
NGWIF

SEQ ID NO: 567 - protein, 5 aa, Human
PQMWD

SEQ ID NO: 568 - protein, 5 aa, Human
PYNLV

SEQ ID NO: 569 - protein, 5 aa, Human
QKHRA

SEQ ID NO: 570 - protein, 5 aa, Human
QMWDF

SEQ ID NO: 571 - protein, 5 aa, Human
QNFRH

SEQ ID NO: 572 - protein, 5 aa, Human
TAKWR

SEQ ID NO: 573 - protein, 5 aa, Human
TDPQM

SEQ ID NO: 574 - protein, 5 aa, Human
VCLGC

SEQ ID NO: 575 - protein, 5 aa, Human
VCYEV

SEQ ID NO: 576 - protein, 5 aa, Human
VMLFC

SEQ ID NO: 577 - protein, 5 aa, Human
VNGWI

SEQ ID NO: 578 - protein, 5 aa, Human
WDFDD

SEQ ID NO: 579 - protein, 5 aa, Human
WIFGT

SEQ ID NO: 580 - protein, 5 aa, Human
WRMVL

SEQ ID NO: 581 - protein, 5 aa, Human
YSPCM

SEQ ID NO: 582 - protein, 487 aa, Human
MSLPNSSCLLEDKMCEGNKTTMASPQLMPLVVVLSTICLVTVGLNLLVLYAVRSERKLHTVGNLYIVSLSVADLIVGAVVMPMNILYLLMSKWSLGRPLCLFWLSMDYVASTASIFSVFILCIDRYRSVQQPLRYLKYRTKTRASATILGAWFLSFLWVIPILGWNHFMQQTSVRREDKCETDFYDVTWFKVMTAIINFYLPTLLMLWFYAKIYKAVRQHCQHRELINRSLPSFSEIKLRPENPKGDAKKPGKESPWEVLKRKPKDAGGGSVLKSPSQTPKEMKSPVVFSQEDDREVDKLYCFPLDIVHMQAAAEGSSRDYVAVNRSHGQLKTDEQGLNTHGASEISEDQMLGDSQSFSRTDSDTTTETAPGKGKLRSGSNTGLDYIKFTWKRLRSHSRQYVSGLHMNRERKAAKQLGFIMAAFILCWIPYFIFFMVIAFCKNCCNEHLHMFTIWLGYINSTLNPLIYPLCNENFKKTFKRILHIRS

SEQ ID NO: 583 - protein, 5 aa, human
AIINF

SEQ ID NO: 584 - protein, 5 aa, human
CEGNK

SEQ ID NO: 585 - protein, 5 aa, human
CNENF

SEQ ID NO: 586 - protein, 5 aa, human
CWIPY

SEQ ID NO: 587 - protein, 5 aa, human
DFYDV

SEQ ID NO: 588 - protein, 5 aa, human
DQMLG

SEQ ID NO: 589 - protein, 5 aa, human
FILCI

SEQ ID NO: 590 - protein, 5 aa, human
FLWVI

SEQ ID NO: 591 - protein, 5 aa, human
FMQQT

SEQ ID NO: 592 - protein, 5 aa, human
FWLSM

SEQ ID NO: 593 - protein, 5 aa, human
GWNHF

SEQ ID NO: 594 - protein, 5 aa, human
HLHMF

SEQ ID NO: 595 - protein, 5 aa, human
HMFTI

SEQ ID NO: 596 - protein, 5 aa, human
HTVGN

SEQ ID NO: 597 - protein, 5 aa, human
IFFMV

SEQ ID NO: 598 - protein, 5 aa, human
ILCWI

SEQ ID NO: 599 - protein, 5 aa, human
LLMSK

SEQ ID NO: 600 - protein, 5 aa, human
LWFYA

SEQ ID NO: 601 - protein, 5 aa, human
MCEGN

SEQ ID NO: 602 - protein, 5 aa, human
MDYVA

SEQ ID NO: 603 - protein, 5 aa, human
MFTIW

SEQ ID NO: 604 - protein, 5 aa, human
MLWFY

SEQ ID NO: 605 - protein, 5 aa, human
NCCNE

SEQ ID NO: 606 - protein, 5 aa, human
NHFMQ

SEQ ID NO: 607 - protein, 5 aa, human
PLCLF

SEQ ID NO: 608 - protein, 5 aa, human
SPWEV

SEQ ID NO: 609 - protein, 5 aa, human
VIAFC

SEQ ID NO: 610 - protein, 5 aa, human
WFYAK

SEQ ID NO: 611 - protein, 5 aa, human
WNHFM

SEQ ID NO: 612 - protein, 5 aa, human
WVIPI

SEQ ID NO: 613 - protein, 5 aa, human
YCFPL

SEQ ID NO: 614 - protein, 25 aa, Human
YDVPDYAGGDYKAFDEQKLISEEDL